(19) United States
(12) Patent Application Publication
(10) Pub. No.: US 2008/0214495 A1
(43) Pub. Date: Sep. 4, 2008

(54) Heterocyclic Sulfonamide Derivatives as Inhibitors of Factor Xa

(75) Inventors: Christer Alstermark, Molndal (SE); Kosrat Amin, Molndal (SE); Kjell Andersson, Molndal (SE); Yantao Chen, Molndal (SE); Ulf Fahlander, Molndal (SE); Kevin Michael Foote, Cheshire (GB); Kenneth Granberg, Molndal (SE); Daniel Hovdal, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 11/994,846

(22) PCT Filed: Jul. 5, 2006
(86) PCT No.: PCT/SE2006/000837

§ 371 Date: Jan. 7, 2008

(30) Foreign Application Priority Data
Jul. 8, 2005 (SE) 0501616-7

(51) Int. Cl. A61K 31/501; C07D 401/14; A61K 31/497; A61K 31/496; A61K 31/397; A61P 9/00

(52) U.S. Cl. 514/56; 514/252.02; 544/238; 544/360; 514/253.01; 514/255.05; 544/405; 514/210.2

(57) ABSTRACT

The invention relates to compounds of formula (I), wherein R¹ and R³ are independently selected from carbon and nitrogen; R² is oxo or thioxo; n is 0, 1 or 2; each R¹⁰ is independently selected from hydrogen and C₁₋₃alkyl; R⁴ and R⁵ are each selected from carbon and nitrogen, wherein at least one of R⁴ and R⁵ is nitrogen; R⁶ is hydrogen or oxo; R⁷ is an aliphatic, partially saturated or aromatic carbocyclic ring, said carbocyclic ring having 0, 1 or 2 hetero nitrogen; m is 0, 1 or 2; each R¹¹ is independently selected from hydrogen, hydroxy, oxo, C₁₋₅alkyl, carboxy, hydroxyC₁₋₅alkyl, carboxyC₁₋₅alkyl, C₁₋₅alkoxy-oxoC₁₋₅alkyl, carbamoyl, C₁₋₅alkylcarbamoyl, di (C₁₋₅ alkyl)carbamoyl, carbamoylC₁₋₄alkyl,C₁₋₅alkylcarbamoylC₁₋₄alkyl, di(C₁₋₅alkyl)carbamoylC₁₋₄alkyl, hydroxyC₁₋₅alkylcarbamoyl, C₁₋₅ alkoxyC₁₋₅alkylcarbamoyl, hydroxyC₁₋₅alkylcarbamoylC₁₋₄alkyl, C₁₋₅alkoxyC₁₋₅ alkylcarbamoylC₁₋₄ alkyl, CONR⁸⁰(CH₂)ₓS(O)ₚR90, CONH(CH₂)_{q}NR¹⁰⁰R¹⁰⁰, —C₁₋₅ alkyl-Y¹, —COOCHR¹⁷⁰R¹⁸⁰ and —CON R¹⁷⁰R¹⁸⁰; R⁸ is a bond, C₁₋₄alkylene or C₂₋₆alkenylene; R⁹ is an aromatic ring system having 0, 1 or 2 hetero atoms; wherein R⁹ is substituted by 0 or 1 halogen; or a pharmaceutically acceptable salt thereof, said compounds possess antithrombotic and anticoagulant properties and are accordingly useful in methods of treatment of humans or animals. The invention also relates to processes for the preparation of the compounds, to their use, to pharmaceutical compositions comprising them, to their use in the manufacture of medicaments for use in the production of an antithrombotic or anticoagulant effect, and to combinations comprising them.

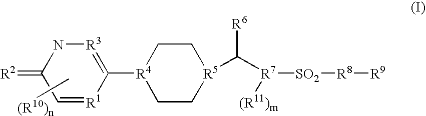

The invention relates to novel heterocyclic derivatives, or pharmaceutically-acceptable salts thereof, which possess antithrombotic and anticoagulant properties and are accordingly useful in methods of treatment of humans or animals. The invention also relates to processes for the preparation of the heterocyclic derivatives, to their use, to pharmaceutical compositions comprising them, to their use in the manufacture of medicaments for use in the production of an antithrombotic or anticoagulant effect, and to combinations comprising them.

The antithrombotic and anticoagulant effect produced by the compounds of the invention is believed to be attributable to their strong inhibitory effect against the activated coagulation protease known as Factor Xa. Factor Xa is one of a cascade of proteases involved in the complex process of blood coagulation. The protease known, as thrombin is the final protease in the cascade and Factor Xa is the preceding protease which cleaves prothrombin to generate thrombin.

Certain compounds are known to possess Factor Xa inhibitory properties and the field has been reviewed by B.-Y. Zhu, R. M. Scarborough, Current Opinion in Cardiovascular, Pulmonary & Renal Investigational Drugs, 1999, 1(1), 63-88. Thus it is known that two proteins, one known as recombinant antistasin (r-ATS) and the other known as recombinant tick anticoagulant protein (r-TAP), are specific direct Factor Xa inhibitors which possess antithrombotic properties in various animal models of thrombotic disease.

It is also known that certain nonpeptidic compounds possess Factor Xa inhibitory properties. Of the low molecular weight inhibitors mentioned in the review by B.-Y. Zhu and R. M. Scarborough, many inhibitors possess a strongly basic group such as an amidinophenyl or amidinonaphthyl group.

We have now found that certain heterocyclic derivatives possess Factor Xa inhibitory activity. Many of the compounds of the present invention also possess the advantage of being selective Factor Xa inhibitors, that is the enzyme Factor Xa is inhibited strongly at concentrations of test compound which do not inhibit or which inhibit to a lesser extent the enzyme thrombin which is also a member of the blood coagulation enzymatic cascade.

The compounds of the present invention possess activity useful in the treatment or prevention of a variety of medical disorders where anticoagulant therapy is indicated, for example in the treatment or prevention of thrombotic conditions such as coronary artery and cerebrovascular disease. Further examples of such medical disorders include various cardiovascular and cerebrovascular conditions such as myocardial infarction, the rupture of atherosclerotic plaques, venous or arterial thrombosis, coagulation syndromes, vascular injury including reocclusion and restenosis following angioplasty and coronary artery bypass surgery, thrombus formation after the application of blood vessel operative techniques or after general surgery such as hip replacement surgery, the introduction of artificial heart valves or on the recirculation of blood, cerebral infarction, cerebral thrombosis, stroke, cerebral embolism, pulmonary embolism, ischemia and angina (including unstable angina).

The compounds of the invention are also useful as inhibitors of blood coagulation in an ex vivo situation such as, for example, the storage of whole blood or other biological samples suspected to contain Factor Xa and in which coagulation is detrimental.

WO 98/21188 describes a range of Factor Xa inhibitors. Further particular examples of this type of compound including 1-(5-chloroindol-2-ylsulphonyl)-4-[4-(6-oxo-1H-pyridazin-3-yl)benzoyl]piperazine are described in WO 99/57113. The applicants have found however, that by further derivatising the compounds of this type, enhanced properties may be obtained.

The present invention provides a compound of formula (I)

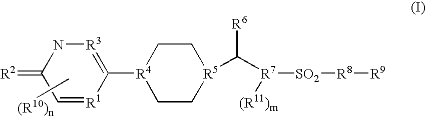

wherein R¹ and R³, are independently selected from carbon and nitrogen;
R² is oxo or thioxo;
n is 0, 1 or 2;
each R¹⁰ is independently selected from hydrogen, halogen and C₁₋₃alkyl;
R⁴ and R⁵ are each selected from carbon and nitrogen, wherein at least one of R⁴ and R⁵ is nitrogen;
R⁶ is hydrogen or oxo;
R⁷ is an aliphatic, partially saturated or aromatic carbocyclic ring, said carbocyclic ring having 0, 1 or 2 hetero nitrogen;
m is 0, 1 or 2;
each R¹¹ is independently selected from hydrogen, hydroxy, oxo, C₁₋₅alkyl, carboxy, hydroxyC₁₋₅alkyl, carboxyC₁₋₅alkyl, C₁₋₅alkoxyoxoC₁₋₅alkyl, carbamoyl, C₁₋₅alkylcarbamoyl, di(C₁₋₅alkyl)carbamoyl, carbamoylC₁₋₄alkyl, C₁₋₅alkylcarbamoylC₁₋₄alkyl, di(C₁₋₅alkyl)carbamoylC₁₋₄alkyl, hydroxyC₁₋₅alkylcarbamoyl, C₁₋₅alkoxyC₁₋₅alkylcarbamoyl, hydroxyC₁₋₅alkylcarbamoylC₁₋₄alkyl, C₁₋₅alkoxyC₁₋₅alkylcarbamoylC₁₋₄alkyl,
—CONR⁸⁰(CH₂)ₓS(O)ₚR⁹⁰, —CONH(CH₂)_{q}NR¹⁰⁰R¹¹⁰, —C₁₋₅alkyl-Y¹, —COOCHR¹⁷⁰R¹⁸⁰ and —CON R¹⁷⁰ R¹⁸⁰:

wherein x represents an integer 0 to 4;

p is 0, 1 or 2;

q represents an integer 2 to 4;

R⁸⁰ represents hydrogen or C₁₋₃alkyl;

R⁹⁰ represents C₁₋₅alkyl or phenyl; or

R⁸⁰ and R⁹⁰ may together form a C₁₋₅alkylene group;

R¹⁰⁰ and R¹¹⁰ independently represent hydrogen, C₁₋₅alkyl, phenyl, C₁₋₅alkylphenyl, S(O)ₚR⁹⁰, COR¹²⁰ or a 5- or 6-membered monocyclic heteroaryl ring containing up to 3 heteroatoms selected from nitrogen, oxygen and sulphur;

R¹²⁰ represents hydrogen, C₁₋₅alkyl or phenyl;

Y¹ represents S(O)ₚR⁹⁰, NHS(O)₂R⁹⁰, NHCOR¹³⁰, O(CH₂)ᵣR¹⁴⁰, azetidino, pyrrolidin-1-yl, piperidino, morpholino, thiamorpholino, 1-oxothiamorpholino, 1,1-dioxothiamorpholino, piperazin-1-yl or C₁₋₅alkylamino,

R¹³⁰ represents C₁₋₅alkyl, phenyl or C₁₋₅alkylphenyl;

r represents an integer 1 to 4;

when r represents an integer 2 to 4, R¹⁴⁰ represents hydroxy, C₁₋₅alkylalkoxy, carboxy, C₁₋₅alkoxycarbonyl, S(O)ₚR⁹⁰ or NR¹⁵⁰R¹⁶⁰; and when r represents 1, R¹⁴⁰ represents carboxy or C₁₋₅alkoxycarbonyl;

wherein any phenyl group within R¹¹ is independently substituted by 0, 1 or 2 substituents selected from halogeno, trifluoromethyl, cyano, C₁₋₅alkyl and C₁₋₅alkoxy;

R¹⁵⁰ and R¹⁶⁰ independently represent hydrogen or C₁₋₅alkyl;
R¹⁷⁰ and R¹⁸⁰ are independently selected from hydrogen, C₁₋₆alkyl, C₄₋₇cycloalkyl, C₂₋₆alkenyl, R¹⁷⁰ and R¹⁸⁰ may form, along with the carbon to which they are attached, a 4-,5-, 6- or 7-membered carbocyclic ring which contains 0, 1 or 2 heteroatoms selected from nitrogen, oxygen and sulphur, or R¹⁷⁰ and R¹⁸⁰ may form, along with the nitrogen to which they are attached, a 4-,5-, 6- or 7-membered heterocyclic ring which contain in addition to the nitrogen atom present 0, 1 or 2 additional heteroatoms selected from nitrogen, oxygen and sulphur, wherein each R¹⁷⁰, R¹⁸⁰ or any of said rings formed by R¹⁷⁰ and R¹⁸⁰ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl;
R⁸ is a bond, C₁₋₄alkylene or C₂₋₆alkenylene;
R⁹ is an aromatic ring system having 0, 1 or 2 hetero atoms;
wherein R⁹ is substituted by 0 or 1 halogen;
or a pharmaceutically acceptable salt thereof.

In this specification the term “alkyl” includes both straight and branched chain alkyl groups but references to individual alkyl groups such as “propyl” are specific for the straight chain version only. An analogous convention applies to other generic terms.

It is to be understood that certain of the compounds of the formula (I) defined above can exist in solvated as well as unsolvated forms such as, for example, hydrated forms. It is to be understood that the invention encompasses all such solvated forms, which possess Factor Xa inhibitory activity.

It is further to be understood that, insofar as certain of the compounds of the formula (I) defined above may exist in optically active or racemic forms by virtue of one or more asymmetric carbon atoms, the invention encompasses any such optically active or racemic form which possesses Factor Xa inhibitory activity. The synthesis of optically active forms may be carried out by standard techniques of organic chemistry well known in the art, for example by synthesis from optically active starting materials or by resolution of a racemic form.

Further, “tautomer” or “tautomerism” refers to the coexistence of two (or more) compounds that differ from each other only in the position of one (or more) mobile atoms and in electron distribution, i.e. different tautomeric forms. An example may be keto-enol tautomers.

Moreover, it is also to be understood that, insofar as certain of the compounds of the formula (I) defined above may exist in various tautomeric forms, the invention encompasses any such tautomeric forms which possesses Factor Xa inhibitory activity.

Compounds of the invention are potent inhibitors of Factor Xa, and may have improved selectivity over oxido squalene cyclase, better solubility and/or less cytochrome P 450 (CYP₄₅₀) inhibition and/or Caco2-permeability than some related compounds. Caco2 is a cell line which mimics transport over the gut wall.

Suitable values in the compound of formula (I):
for halogen: fluoro, chloro, bromo, iodo;
for C₁₋₃alkyl (also as in e.g. oxoC₁₋₃alkyl): methyl, ethyl, propyl, isopropyl;
for C₁₋₄alkyl (also as in e.g. oxoC₁₋₄alkyl): methyl, ethyl, propyl, isopropyl, n-butyl, secbutyl, isobutyl, tertbutyl;
for C₁₋₅alkyl (also as in e.g. oxoC₁₋₅alkyl): C₁₋₄alkyl (as above), C₁₋₃alkyl (as above), n-butyl, isobutyl, pentyl, 2-pentyl, 3-pentyl, 2-methyl-1-butyl, isopentyl, neopentyl, 3-methyl-2-butyl, 2-methyl-2-butyl;
for C₁₋₃alkoxy: methoxy, ethoxy, propoxy, isopropoxy;
for C₁₋₄alkoxy: C₁₋₃alkoxy (as above), n-butoxy, secbutoxy, isobutoxy, terbutoxy;
for C₁₋₅alkoxy: C₁₋₄alkoxy (as above), C₁₋₃alkoxy (as above), pentoxy, 2-pentoxy, 3-pentoxy, 2-methyl-1-butoxy, isopentoxy, neopentoxy, 3-methyl-2-butoxy, 2-methyl-2-butoxy;
   for 4-,5-, 6- or 7-membered heterocyclic ring: azetidine, pyrrolidine, morpholine, piperazine, azepane, [1,4]-diazepane, tetrahydro-pyran, or piperidin.

Moreover, the term “oxido” denotes a —O-group (ion) and the term “carbamoyl” denotes a H₂N—C(O)-group.

In an embodiment of the invention a compound of formula (I) is disclosed wherein R¹ is nitrogen.

A further embodiment of the invention discloses a compound of formula (I) wherein R³ is nitrogen.

In a further embodiment of the invention a compound of formula (I) is disclosed wherein R² is oxo.

A further embodiment of the invention discloses a compound of formula (I) wherein n is 0 or 1.

In still a further embodiment of the invention a compound of formula (I) is disclosed wherein one of R¹⁰ is hydrogen.

In even a further embodiment of the invention a compound of formula (I) is disclosed wherein one of R¹⁰ is C₁₋₃alkyl, e.g. methyl, ethyl, or propyl.

A further embodiment of the invention discloses a compound of formula (I) wherein R⁴ is nitrogen.

In a further embodiment of the invention a compound of formula (I) is disclosed wherein R⁵ is nitrogen.

In an embodiment of the invention a compound of formula (I) is disclosed wherein both R⁴ and R⁵ are nitrogen.

A further embodiment of the invention discloses a compound of formula (I) wherein R⁶ is hydrogen.

In an embodiment of the invention a compound of formula (I) is disclosed wherein R⁶ is oxo.

A further embodiment of the invention discloses a compound of formula (I) wherein R⁷ is an aliphatic carbocyclic ring.

In a further embodiment of the invention a compound of formula (I) is disclosed wherein R⁷ is a partially saturated carbocyclic ring.

In still a further embodiment of the invention a compound of formula (I) is disclosed wherein R⁷ is an aromatic carbocyclic ring.

In even a further embodiment of the invention a compound of formula (I) is disclosed wherein said carbocyclic ring has 0 hetero nitrogen.

In a further embodiment of the invention a compound of formula (I) is disclosed wherein said carbocyclic ring has 1 hetero nitrogen.

In even a further embodiment of the invention a compound of formula (I) is disclosed wherein said carbocyclic ring has 2 hetero nitrogens.

A further embodiment of the invention discloses a compound of formula (I) wherein R⁷ is a carbocyclic ring of formula (Ia)

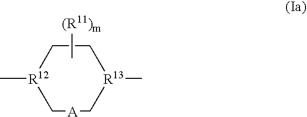

wherein A is a single bond or a double bond, and said hetero nitrogen or nitrogens is/are positioned at R¹² and/or R¹³.

In a further embodiment of the invention a compound of formula (I) is disclosed wherein R⁷ is a carbocyclic ring of formula (Ia) and A is a single bond.

A further embodiment of the invention discloses a compound of formula (I) wherein R⁷ is a carbocyclic ring of formula (Ia) and said hetero nitrogens are positioned at R¹² and R¹³, respectively.

In a further embodiment of the invention a compound of formula (I) is disclosed wherein R⁷ is a carbocyclic ring of formula (Ia) and said hetero nitrogen is positioned at R¹³.

A further embodiment of the invention discloses a compound of formula (I) where each R¹¹ is independently selected from hydrogen, hydroxy, oxo, C₁₋₅alkyl, carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl, C₁₋₅alkoxyC₁₋₅alkylcarbamoyl, —COOCHR¹⁷⁰R¹⁸⁰ and —CON R¹⁷⁰ R¹⁸⁰:
wherein

R¹⁷⁰ and R¹⁸⁰ are independently selected from hydrogen, C₁₋₆alkyl, C₄₋₇cycloalkyl, C₂₋₆alkenyl, R¹⁷⁰ and R¹⁸⁰ may form, along with the carbon to which they are attached, a 4-, 5-, 6- or 7-membered carbocyclic ring which contains 0, 1 or 2 heteroatoms selected from nitrogen, oxygen and sulphur, or R¹⁷⁰ and R¹⁸⁰ may form, along with the nitrogen to which they are attached, a 4-, 5-, 6- or 7-membered heterocyclic ring which contain in addition to the nitrogen atom present 0, 1 or 2 additional heteroatoms selected from nitrogen, oxygen and sulphur, wherein each R¹⁷⁰, R¹⁸⁰ or any of said rings formed by R¹⁷⁰ and R¹⁸⁰ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl.

In a further embodiment of the invention a compound of formula (I) is disclosed wherein one R¹¹ is oxo, and at least one further R¹¹ is selected from hydroxy, oxo, C₁₋₅alkyl, carboxy, hydroxyC₁₋₅alkyl, carboxyC₁₋₅alkyl, C₁₋₅alkoxyoxoC₁₋₅alkyl, carbamoyl, C₁₋₅alkylcarbamoyl, di(C₁₋₅alkyl)carbamoyl, carbamoylC₁₋₄alkyl, C₁₋₅alkylcarbamoylC₁₋₄alkyl, di(C₁₋₅alkyl)carbamoylC₁₋₄alkyl, hydroxyC₁₋₅alkylcarbamoyl, C₁₋₅alkoxyC₁₋₅alkylcarbamoyl, hydroxyC₁₋₅alkylcarbamoylC₁₋₄alkyl, C₁₋₅alkoxyC₁₋₅alkylcarbamoylC₁₋₄alkyl, —CONR⁸⁰(CH₂)ₓS(O)ₚR⁹⁰, —CONH(CH₂)_{q}NR¹⁰⁰R¹¹⁰, —C₁₋₅alkyl-Y¹, —COOCHR¹⁷⁰R¹⁸⁰ and —CON R¹⁷⁰ R¹⁸⁰:
wherein x represents an integer 0 to 4;
p is 0, 1 or 2;
q represents an integer 2 to 4;
R⁸⁰ represents hydrogen or C₁₋₃alkyl;
R⁹⁰ represents C₁₋₅alkyl or phenyl; or
R⁸⁰ and R⁹⁰ may together form a C₁₋₅alkylene group;
R¹⁰⁰ and R¹¹⁰ independently represent hydrogen, C₁₋₅alkyl, phenyl, C₁₋₅alkylphenyl, S(O)ₚR⁹⁰, COR¹²⁰ or a 5- or 6-membered monocyclic heteroaryl ring containing up to 3 heteroatoms selected from nitrogen, oxygen and sulphur;
R¹²⁰ represents hydrogen, C₁₋₅alkyl, phenyl or C₁₋₅alkylphenyl;
Y¹ represents S(O)ₚR⁹⁰, NHS(O)₂R⁹⁰, NHCOR¹³⁰, O(CH₂)ᵣR¹⁴⁰, pyrrolidin-1-yl, piperidino, morpholino, thiamorpholino, 1-oxothiamorpholino, 1,1-dioxothiamorpholino or piperazin-1-yl,
R¹³⁰ represents C₁₋₅alkyl, phenyl or C₁₋₅alkylphenyl;
r represents an integer 1 to 4;
when r represents an integer 2 to 4, R¹⁴⁰ represents hydroxy, C₁₋₅alkylalkoxy, carboxy, C₁₋₅alkoxycarbonyl, S(O)ₚR⁹⁰ or NR¹⁵⁰R¹⁶⁰; and when r represents 1, R¹⁴⁰ represents carboxy or C₁₋₅alkoxycarbonyl;
wherein any phenyl group within R¹¹ is independently substituted by 0, 1 or 2 substituents selected from halogeno, trifluoromethyl, cyano, C₁₋₅alkyl and C₁₋₅alkoxy;
R¹⁵⁰ and R¹⁶⁰ independently represent hydrogen or C₁₋₅alkyl;

R¹⁷⁰ and R¹⁶⁰ are independently selected from hydrogen, C₁₋₆alkyl, C₄₋₇cycloalkyl, C₂₋₆alkenyl, R¹⁷⁰ and R¹⁸⁰ may form along with the carbon to which they are attached a 4-, 5-, 6- or 7-membered carbocyclic ring which contains 0, 1 or 2 he teroatoms selected from nitrogen, oxygen and sulphur, or R¹⁷⁰ and R¹⁸⁰ may form along with the nitrogen to which they are attached a 4-, 5-, 6- or 7-membered heterocyclic ring which contain in addition to the nitrogen atom present 0, 1 or 2 additional heteroatoms selected from nitrogen, oxygen and sulphur, wherein each R¹⁷⁰, R¹⁸⁰ or any of said rings formed by R¹⁷⁰ and R¹⁵⁰ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl.

In even a further embodiment of the invention a compound of formula (I) is disclosed wherein one R¹¹ is oxo and at least one further R¹¹ is selected from hydroxy, C₁₋₃alkyl, carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl, C₁₋₅alkoxyC₁₋₅alkylcarbamoyl, —CONR⁸⁰(CH₂)ₓS(O)ₚR⁹⁰, —CONH(CH₂)_{q}NR¹⁰⁰R¹¹⁰, —C₁₋₅alkyl-Y¹, —COOCHR¹⁷⁰R¹⁸⁰ and —CON R¹⁷⁰R¹⁸⁰:
wherein x represents an integer 0 to 4;
p is 0, 1 or 2;
q represents an integer 2 to 4;
R⁸⁰ represents hydrogen or C₁₋₃alkyl;
R⁹⁰ represents C₁₋₅alkyl or phenyl; or
R⁸⁰ and R⁹⁰ may together form a C₁₋₅alkylene group;
R¹⁰⁰ and R¹¹⁰ independently represent hydrogen, C₁₋₅alkyl, phenyl, C₁₋₅alklylphenyl, S(O)ₚR⁹⁰, COR¹²⁰ or a 5- or 6-membered monocyclic heteroaryl ring containing up to 3 heteroatoms selected from nitrogen, oxygen and sulphur;
R¹²⁰ represents hydrogen, C₁₋₅alkyl, phenyl or C₁₋₅alkylphenyl;
Y¹ represents S(O)ₚR⁹⁰, NHS(O)₂R⁹⁰, NHCOR¹³⁰, O(CH₂)ᵣR¹⁴⁰, pyrrolidin-1-yl, piperidino, morpholino, thiamorpholino, 1-oxothiamorpholino, 1,1-dioxothiamorpholino or piperazin-1-yl;
R¹³⁰ represents C₁₋₅alkyl, phenyl or C₁₋₅alkylphenyl;
r represents an integer 1 to 4;
when r represents an integer 2 to 4, R¹⁴⁰ represents hydroxy, C₁₋₅alkylalkoxy, carboxy, C₁₋₅alkoxycarbonyl, S(O)ₚR⁹⁰ or NR¹⁵⁰R¹⁶⁰; and when r represents 1, R¹⁴⁰ represents carboxy or C₁₋₅alkoxycarbonyl;
wherein any phenyl group within R¹¹ is independently substituted by 0, 1 or 2 substituents selected from halogeno, trifluoromethyl, cyano, C₁₋₅alkyl and C₁₋₅alkoxy;
R¹⁵⁰ and R¹⁶⁰ independently represent hydrogen or C₁₋₅alkyl;

R¹⁷⁰ and R¹⁸⁰ are independently selected from hydrogen, C₁₋₆alkyl, C₄₋₇cycloalkyl, C₂₋₆alkenyl, R¹⁷⁰ and R¹⁸⁰ may form along with the carbon to which they are attached a 4-, 5-, 6- or 7-membered carbocyclic ring which contains 0, 1 or 2 heteroatoms selected from nitrogen, oxygen and sulphur, or R¹⁷⁰ and R¹⁸⁰ may form along with the nitrogen to which they are attached a 4-, 5-, 6- or 7-membered heterocyclic ring which contain in addition to the nitrogen atom present 0, 1 or 2 additional heteroatoms selected from nitrogen, oxygen and sulphur, wherein each R¹⁷⁰, R¹⁸⁰ or any of said rings formed by R¹⁷⁰ and R¹⁸⁰ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl.

In still a further embodiment of the invention a compound of formula (I) is disclosed wherein one R¹¹ is oxo and at least one further R¹¹ is selected from hydroxy, C₁₋₃alkyl, carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl, C₁₋₅alkoxyC₁₋₅alkylcarbamoyl, —COOCHR¹⁷⁰R¹⁸⁰ and —CON R¹⁷⁰R¹⁸⁰:

R¹⁷⁰ and R¹⁸⁰ are independently selected from hydrogen, C₁₋₆alkyl, C₄₋₇cycloalkyl, C₂₋₆alkenyl, R¹⁷⁰ and R¹⁸⁰ may form along with the carbon to which they are attached a 4-, 5-, 6- or 7-membered carbocyclic ring which contains 0, 1 or 2 heteroatoms selected from nitrogen, oxygen and sulphur, or R¹⁷⁰ and R¹⁸⁰ may form along with the nitrogen to which they are attached a 4-, 5-, 6- or 7-membered heterocyclic ring which contain in addition to the nitrogen atom present 0, 1 or 2 additional heteroatoms selected from nitrogen, oxygen and sulphur, wherein each R¹⁷⁰, R¹⁸⁰ or any of said rings formed by R¹⁷⁰ and R¹⁸⁰ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl.

In a further embodiment of the invention a compound of formula (I) is disclosed wherein one R¹¹ is oxo and at least one further R¹¹ is selected from C₁₋₃alkyl, carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl and C₁₋₅alkoxyC₁₋₅alkylcarbamoyl.

In even a further embodiment of the invention a compound of formula (I) is disclosed wherein one R¹¹ is oxo and at least one further R¹¹ is selected from —COOCHR¹⁷⁰R¹⁸⁰ and —CON R¹⁷⁰R¹⁸⁰:

R¹⁷⁰ and R¹⁸⁰ are independently selected from hydrogen, C₁₋₆alkyl, C₄₋₇cycloalkyl, C₂₋₆alkenyl, R¹⁷⁰ and R¹⁸⁰ may form along with the carbon to which they are attached a 4-, 5-, 6- or 7-membered carbocyclic ring which contains 0, 1 or 2 heteroatoms selected from nitrogen, oxygen and sulphur, or R¹⁷⁰ and R¹⁸⁰ may form along with the nitrogen to which they are attached a 4-, 5-, 6- or 7-membered heterocyclic ring which contain in addition to the nitrogen atom present 0 or 1 additional hetero oxygen, wherein each R¹⁷⁰, R¹⁸⁰ or any of said rings formed by R¹⁷⁰ and R¹⁸⁰ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl.

A further embodiment of the invention discloses a compound of formula (I) wherein R⁶ is oxo.

In a further embodiment of the invention a compound of formula (I) is disclosed wherein each R¹¹ is independently selected from hydrogen, hydroxy, C₁₋₃alkyl, carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl, and C₁₋₅alkoxyC₁₋₅alkylcarbamoyl.

In still a further embodiment of the invention a compound of formula (I) is disclosed wherein wherein one R¹¹ is hydroxy.

In even a further embodiment of the invention a compound of formula (I) is disclosed wherein m is 0.

In an embodiment of the invention a compound of formula (I) is disclosed wherein R⁸ is a bond.

A further embodiment of the invention discloses a compound of formula (I) wherein R⁸ is a C₂₋₄alkenylene.

In a further embodiment of the invention a compound of formula (I) is disclosed wherein R⁹ is an aromatic ring system having 0, 1 or 2 hetero atoms, which hetero atoms are independently selected from nitrogen, oxygen and sulphur.

A further embodiment of the invention discloses a compound of formula (I) wherein R⁹ is an aromatic ring system and said aromatic ring system is an aromatic ring.

In a further embodiment of the invention a compound of formula (I) is disclosed wherein R⁹ is an aromatic ring system and said aromatic ring has 1 hetero sulphur.

A further embodiment of the invention discloses a compound of formula (I) wherein R⁹ is an aromatic ring system and said aromatic ring system is a fused bicyclic system comprising at least one benzene ring.

In still a further embodiment of the invention a compound of formula (I) is disclosed wherein said fused bicyclic system has 0 hetero atom.

In even a further embodiment of the invention a compound of formula (I) is disclosed wherein said fused bicyclic system has 1 hetero nitrogen.

A further embodiment of the invention discloses a compound of formula (I) wherein R⁹ is substituted by 0 or 1 halogen, e.g. chloro or bromo.

In an embodiment of the invention a compound of formula (I) is disclosed which is

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid,

(R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid methyl ester,

6-{4-[4-(3-Chloro-1H-indole-6-sulfonyl)-3-hydroxy-piperazine-1-carbonyl]-piperidin-1-yl}-2-methyl-2H-pyridazin-3-one,

6-{4-[4-(3-Chloro-1H-indole-6-sulfonyl)-2-hydroxy-piperazine-1-carbonyl]-piperidin-1-yl}-2-methyl-2H-pyridazin-3-one,

6-{4-[4-(3-Chloro-1H-indole-6-sulfonyl)-2-oxo-piperazin-1-ylmethyl]-piperidin-1-yl}-2-methyl-2H-pyridazin-3-one,

4-[4-(3-Chloro-1H-indole-6-sulfonyl)-piperazine-1-carbonyl]-5′-methyl-3,4,5,6-tetrahydro-2H,1′H-[1,3′]bipyridinyl-6′-one,

5-{4-[4-(3-Chloro-1H-indole-6-sulfonyl)-piperazine-1-carbonyl]-piperidin-1-yl}-3-methyl-1H-pyrazin-2-one,

6-{4-[4-(3-Chloro-1H-indole-6-sulfonyl)-piperazine-1-carbonyl]-piperidin-1-yl}-2-methyl-2H-pyridazin-3-one,

6-{4-[4-(1H-Indole-6-sulfonyl)-piperazine-1-carbonyl]-piperidin-1-yl}-2-methyl-2H-pyridazin-3-one,

6-{4-[4-(3-Chloro-1H-indole-6-sulfonyl)-piperazine-1-carbonyl]-piperidin-1-yl}-2H-pyridazin-3-one,

6-{4-[4-(3-Chloro-1H-indole-6-sulfonyl)-benzoyl]-piperazin-1-yl}-2-methyl-2H-pyridazin-3-one,

6-{4-[4-(6-Bromo-naphthalene-2-sulfonyl)-benzoyl]-piperidin-1-yl}-2-methyl-2H-pyridazin-3-one,

6-(4-{4-[(E)-2-(5-bromo-thiophen-2-yl)-ethenesulfonyl]-piperazine-1-carbonyl}-piperidin-1-yl)-2H-pyridazin-3-one,

6-(4-{4-[(E)-1-(5-chloro-thiophen-2-yl)-prop-1-ene-2-sulfonyl]-piperazine-1-carbon}-piperidin-1-yl)-2H-pyridazin-3-one,

6-{1-[1-(5-chloro-1H-indole-2-sulfonyl)-piperidine-4-carbonyl]-piperazin-4-yl}-2-methyl-2H-pyridazin-3-one,

6-{1-[1-(5-Chloro-1H-indole-2-sulfonyl)-piperidine-4-carbonyl]-piperidin-4-yl}-2H-pyridazin-3-one,

6-(4-{(S)-4-[(E)-2-(5-chloro-thiophen-2-yl)-ethenesulfonyl]-2-methyl-6-oxo-piperazin-1-ylmethyl}-piperidin-1-yl)-2-methyl-2H-pyridazin-3-one,

6-(4-{(S)-4-[(E)-2-(5-chloro-thiophen-2-yl)-ethenesulfonyl]-5-methyl-2-oxo-piperazin-1-ylmethyl}-piperidin-1-yl)-2-methyl-2H-pyridazin-3-one,

(R)-4-(5-Chloro-1H-indole-2-sulfonyl)-1-[1-(1-methyl-6-oxo-1,6-dihydro-pyridazin-3-yl)-piperidin-4-ylmethyl]-6-oxo-piperazine-2-carboxylic acid methyl ester,

(R)-4-(5-Chloro-1H-indole-2-sulfonyl)-1-[1-(1-methyl-6-oxo-1,6-dihydro-pyridazin-3-yl)-piperidin-4-ylmethyl]-6-oxo-piperazine-2-carboxylic acid,

(R)-4-(6-Chloro-naphthalene-2-sulfonyl)-1-[1-(1-methyl-6-oxo-1,6-dihydro-pyridazin-3-yl)-piperidin-4-ylmethyl]-6-oxo-piperazine-2-carboxylic acid methyl ester,

(R)-4-(6-Chloro-naphthalene-2-sulfonyl)-1-[1-(1-methyl-6-oxo-1,6-dihydro-pyridazin-3-yl)-piperidin-4-ylmethyl]-6-oxo-piperazine-2-carboxylic acid,

(R)-4-[(E)-2-(5-Chloro-thiophen-2-yl)-ethenesulfonyl]-1-[1-(1-methyl-6-oxo-1,6-dihydro-pyridazin-3-yl)-piperidin-4-ylmethyl]-6-oxo-piperazine-2-carboxylic acid,

(P)-4-[(E)-2-(5-Chloro-thiophen-2-yl)-ethenesulfonyl]-1-[1-(1-methyl-6-oxo-1,6-dihydro-pyridazin-3-yl)-piperidin-4-ylmethyl]-6-oxo-piperazine-2-carboxylic acid methyl ester,

6-{4-[4-(6-Chloro-naphthalene-2-sulfonyl)-piperazine-1-carbonyl]-piperidin-1-yl}-2-methyl-2H-pyridazin-3-one,

6-(4-{4-[(E)-2-(5-Chloro-thiophen-2-yl)-ethenesulfonyl]-2-oxo-piperazin-1-ylmethyl}-piperidin-1-yl)-2-methyl-2H-pyridazin-3-one,

6-{4-[4-(5-Chloro-1H-indole-2-sulfonyl)-piperazine-1-carbonyl]-piperidin-1-yl}-2-methyl-2H-pyridazin-3-one,

6-(4-{4-[(E)-2-(5-Chloro-thiophen-2-yl)-ethenesulfonyl]-piperazine-1-carbonyl}-piperidin-1-yl)-2-methyl-2H-pyridazin-3-one,

6-{4-[4-(5-Chloro-1H-indole-2-sulfonyl)-3-hydroxy-piperazine-1-carbonyl]-piperidin-1-yl}-2-methyl-2H-pyridazin-3-one,

6-{4-[4-(3-Chloro-1H-indole-6-sulfonyl)-3,4-dihydro-2H-pyrazine-1-carbonyl]piperidin-1-yl}-2-methyl-2H-pyridazin-3-one,

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid dimethylamide,

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid ethylamide,

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-hydroxy-ethyl)-amide,

6-{4-[4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one,

6-{4-[(R)-4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one,

6-{4-[(S)-4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one,

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropylamide,

(R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropylamide, (S)-4-(3-Chloro-1H-indole-6-sulfonyl)-1-[1-(1-methyl-6-oxo-1,6-dihydro pyridazin-3-yl)-piperidin-4-ylmethyl]-6-oxo-piperazine-2-carboxylic acid isopropylamide,

6-{4-[2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one,

6-{4-[(R)-2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one,

6-{4-[(S)-2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one,

6-{4-[4-(3-Chloro-1H-indole-6-sulfonyl)-2-hyrdoxymethyl-6-oxo-piperazin-1ylmethyl]-piperidine-1-yl}-2-methyl-2H-pyridazin-3-one,

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide,

(R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide,

(S)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide,

4-(3-Chloro-1H-indole-6-sulfonyl)-1-[1-(1-methyl-6-oxo-1,6-dihydro-pyridazin-3yl)-piperidin-4-ylmethyl]-6-oxo-piperazine-2-carboxylic acid tert-butyl ester,

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid ethyl ester,

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropyl ester, or

6-[4-({4-[(5-chloro-1H-indol-2-yl)sulfonyl]piperazin-1-yl}carbonyl)piperidin-1-yl]pyridazin-3(2B)-one.

A heterocyclic derivative of formula I, or pharmaceutically acceptable salt thereof, may be prepared by any process known to be applicable to the preparation of related compounds, such as those described in WO 98/21188 and WO 99/57113. Such procedures are provided as a further feature of the invention and are illustrated by the following representative processes in which, unless otherwise stated any functional group, for example amino, aminoalkyl, carboxy, indolyl or hydroxy, is optionally protected by a protecting group which may be removed when necessary.

Necessary starting materials may be obtained by standard procedures of organic chemistry and by reference to the processes used in the Examples.

For instance, the present invention provides a process for preparing a compound of formula (I) or a pharmaceutically acceptable salt thereof, which comprises the reaction, conveniently in the presence of a suitable base, of an amine of formula (II), wherein R7a is a secondary amine part of a saturated or partially saturated heterocycle,

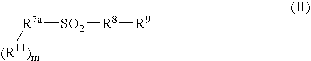

with a carboxylic acid of the formula (III)

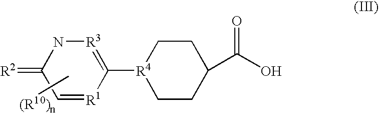

wherein R-groups, m and n are defined as above in relation to formula (I), or a suitably reactive derivative.

Alternatively, a carboxylic acid derivative of formula (IV), or a suitably reactive derivative thereof

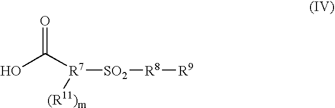

may be reacted with an amine such as (V)

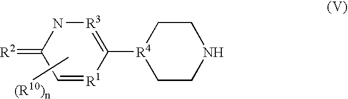

wherein R-groups, m and n are defined as above in relation to formula (I).

A suitable reactive derivative of an carboxylic acid of the formula (III) and (IV) is, for example, an acyl halide, for example an acyl chloride formed by the reaction of the acid and an inorganic acid chloride, for example thionyl chloride; a mixed anhydride, for example an anhydride formed by the reaction of the acid with a chloroformate such as isobutyl chloroformate or with an activated amide such as 1,1′-carbonyldiimidazole; an active ester, for example an ester formed by the reaction of the acid and a phenol such as pentafluorophenol, an ester such as pentafluorophenyl trifluoroacetate or an alcohol such as N-hydroxybenzotriazole or N-hydroxysuccinimide; an acyl azide, for example an azide formed by the reaction of the acid and an azide such as diphenylphosphoryl azide; an acyl s cyanide, for example a cyanide formed by the reaction of an acid and a cyanide such as diethylphosphoryl cyanide; or the product of the reaction of the acid and a carbodiimide such as N,N′ dicyclohexylcarbodiimide or N-(3 dimethylaminopropyl) N′ ethyl-carbodiimide.

The reaction is conveniently carried out in the presence of a suitable base such as, for l0 example, an alkali or alkaline earth metal carbonate, also preferably carried out in a suitable inert solvent or diluent, for example methylene chloride or N,N-dimethylformamide, and at a temperature in the range, for example, −78° C. to 150° C., conveniently at or near ambient temperature

Compounds of formula (VI)

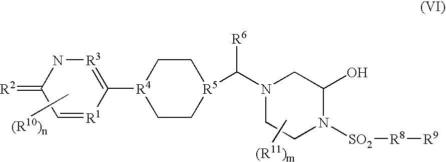

are suitably prepared by oxidative cleavage of the exocyclic double bond of formula (VII), wherein the possible positioning of (R¹¹)ₘ corresponds to the possible positions of (R¹¹)ₘ in the compound of formula (VI), the R-groups, n and m are as defined above in relation to formula (I). The in situ formed aldehyde spontaneously cyclize to form the more stable hemiaminal.

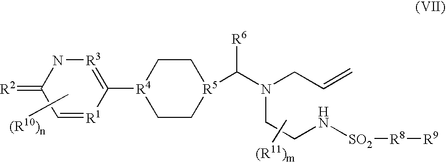

Typically, this reaction is carried out by reacting the compound of formula (V) with oxidazing agent such as sodium periodate/osmium tetroxide or ozone/dimethyl sulfide, also preferably carried out in a suitable inert solvent or diluent, for example tetrahydrofuran, methylene chloride, dioxane and at a temperature in the range, for example, −78° C. to 75° C., conveniently at or near ambient temperature.

Compounds of formula (VIII), wherein the indolyl ring is substituted at C-3 by a halogen such as chloro or bromo,

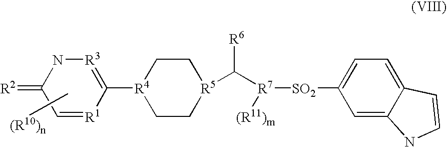

are prepared from compounds of formula (VIII), wherein the R-groups, n and m are as defined above in relation to formula (I).

This reaction is carried out using the corresponding halogen succinimide in an inert solvent like dichloromethane or N,N-dimethylformamide at a temperature in the range −50° C.-100° C., conveniently at or near ambient temperature.

Compounds of formula (X)

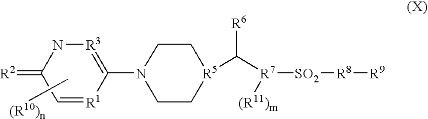

are formed by reacting amine derivatives of formula (XI),

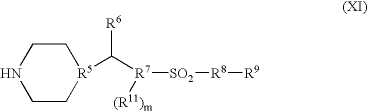

with an structure of formula (XII) wherein A¹ denotes a leaving group typically halogen

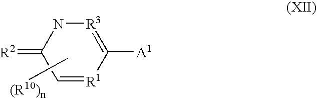

wherein R-groups, m and n are defined as above in relation to formula (I), or a suitably reactive derivative.

The above said reaction is conveniently performed by heating, preferably using microvawe irradiation. Alterantive conditions may involve the use of transition metal catalysis, eg a Pd(II) or Pd(0) metal complex in an inert solvent such as tetrahydrofuran or N,N-dimethylformamide with or without heating or microvawe irradiation.

The preperation of derivatives of formula (XIII)

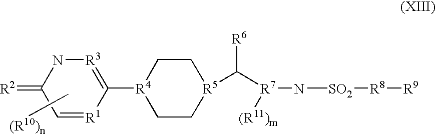

Are prepared by reaction a sulfonyl chloride derivative of formula (XIV),

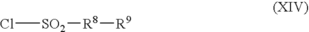

with an amine of formula (XV),

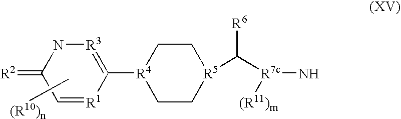

wherein the R-groups, n and m are as defined above in relation to formula (I).

This reaction is carried out using a base such as N,N-dimethyl aminopyridine, diisopropylethyl amine in inert solvents, typically dichloromethane and N,N-dimethylformamide at a temperature in the range −50° C.-100° C., conveniently at or near ambient temperature.

In an alternative embodiment, amide derivatives from the exocyclic carboxylic acid of formula (XVI), or a reactive derivative thereof,

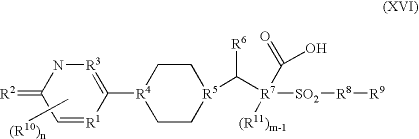

are prepared using conditions such as those described above for the conversion of (II) to (III), wherein the R-groups, n and m are as defined above in relation to formula (I).

In an alternative embodiment, ester derivatives from the exocyclic carboxylic acid of formula (XVI) or a reactive derivative thereof, wherein the R-groups, are as defined above in relation to formula (I), are prepared using standard conditions following references found in Comprehensive Organic Transformations by Richard C. Larock. For example, for example treatment of (IX) in an readily available alholic solvent using acid catalysis, for example, using by saturation of the solvent by gaseous hydrochloric acid, furnish the corresponding ester derivatives. In case of hindered alcohols N,N-dimethylformamide dialkyl acetal is useful.

Compounds of formula (XVII)

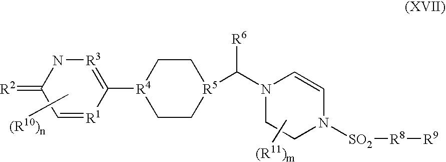

are prepared from compounds of formula (VI), wherein the R-groups, n and m are as defined above in relation to formula (I).

This reaction is carried out using acidic conditions conveniently in alcoholic solvents, typically methanol at a temperature in the range −50° C.-100° C., conveniently at or near ambient temperature

Compounds of formula (XVIII)

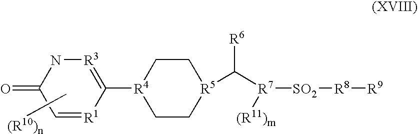

are prepared from compounds of formula (XIX), wherein the R-groups, n and m are as defined above in relation to formula (I) and Y is typically a halogen such as chloro or bromo.

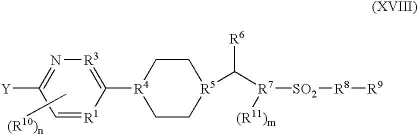

This reaction is carried out using acidic conditions conveniently in alcoholic solvents, typically methanol at a temperature in the range −50° C.-200° C.

Compounds of formula (XIX)

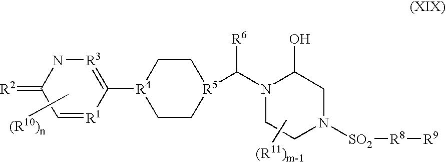

are suitably prepared by oxidative cleavage of the exocyclic double bond of formula (XX), wherein the possible positioning of (R¹¹)ₘ₋₁ corresponds to the possible positions of (R¹¹)ₘ₋₁ in the compound of formula (XIX), the R-groups, n and m are as defined above in relation to formula (I). The in situ formed aldehyde spontaneously cyclize to form the more stable hemiaminal.

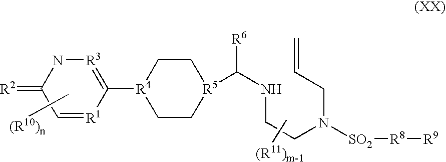

Typically, this reaction is carried out as described for the conversion of (VII) to (VI).

When an optically active form of a compound of the formula (I) is required, it may be obtained, for example, by carrying out one of the aforesaid procedures using an optically active starting material or by resolution of a racemic form of said compound using a conventional procedure, for example by the formation of diastereomeric salts, use of chromatographic techniques, conversion using stereospecific enzymatic processes, or by addition of temporary extra chiral group to aid separation.

The invention also relates to a process for preparing a compound of formula (I) which process comprises either

(a) reacting an amine of formula (II),

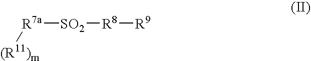

wherein R^{7a} is a secondary amine part of a saturated or partially saturated heterocycle,

with a carboxylic acid of the formula (III);

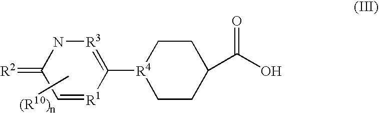

(b) reacting a carboxylic acid derivative of formula (IV), or a suitably reactive derivative thereof

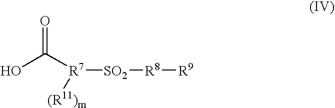

with an amine such as (V);

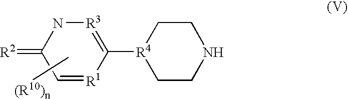

(c) oxidative cleaving the exocyclic double bond of formula (VII);

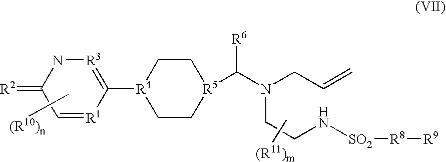

(d) preparing a compound of formula (VIII),

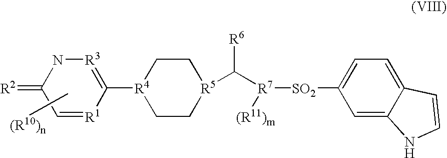

wherein the indolyl ring is substituted at C-3 by a halogen such as chloro or bromo, from compounds of formula (VIII) by using the corresponding halogen succinimide;

(e) reacting an amine derivative of formula (XI),

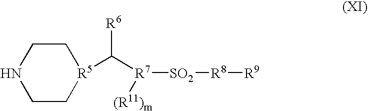

with an structure of formula (XII)

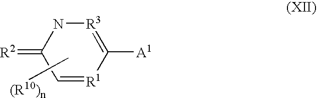

wherein A¹ denotes a leaving group typically halogen;

(f) reacting a sulfonyl chloride derivative of formula (XIV),

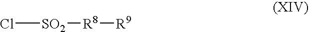

with an amine of formula (XV);

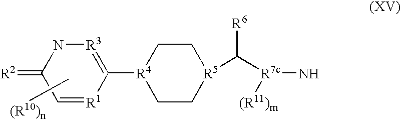

(g) amide derivatives from the exocyclic carboxylic acid of formula (XVI), or a reactive derivative thereof,

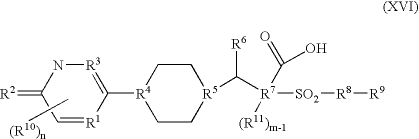

are prepared using conditions such as those described above under (a) for the conversion of (II) to (III);

(h) an ester derivative from the exocyclic carboxylic acid of formula (XVI) or a reactive derivative thereof, are prepared using acid catalysis, for example, using by saturation of the solvent by gaseous hydrochloric acid, and using in case of hindered alcohols N,N-dimethylformamide dialkyl acetal;

(i) treating compounds of formula (VI) in acidic conditions;

(j) treating compounds of formula (XIX),

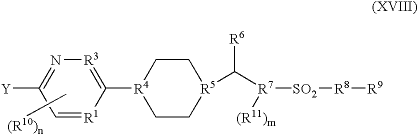

wherein Y is typically a halogen such as chloro or bromo, in acidic conditions; or

(k) oxidative cleavaging of the exocyclic double bond of formula (XX),

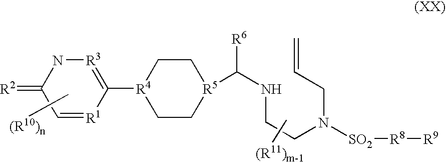

as (c) above.

As stated previously, the compounds of the formula (I) are inhibitors of the enzyme Factor Xa. The effects of this inhibition may be demonstrated using one or more of the standard procedures set out hereinafter:

a) Measurement of Factor Xa Inhibition

The FXa inhibitor potency was measured with a chromogenic substrate method, in a Plato 3300 robotic microplate processor (Rosys AG, CH-8634 Hombrechtikon, Switzerland), using 96-well, half-volume microtiter plates (Costar, Cambridge, Mass., USA; Cat No 3690). Stock solutions of test substance in DMSO (72 μL), 10 mmol/L, alternatively 1 mmol/L were diluted serially 1:3 (24+48 μL) with DMSO to obtain ten different concentrations, which were analyzed as samples in the assay, together with controls and blanks. As control sample melagatran was analysed. The dilutions of each test substance were analyzed consecutively, row-wise on the microtiter plate, with wash-cycles between substances to avoid cross-contamination. First 2 μL of test sample or DMSO for the blank were added, followed by 124 μL of assay buffer (0.05 mol/L Tris-hydrochloric acid pH 7.4 at 37° C., 5 mM CaCl₂, ionic strength 0.15 adjusted with NaCl, 0.1% bovine serum albumin, ICN Biomedicals, Inc, USA, 1 g/L) and 12 μL of chromogenic substrate solution (S-2765, Chromogenix, Mölndal, Sweden) and finally 12 μL of FXa solution (human FXa, Haematologic Technologies Inc., Essec Junction, Vt., USA), in buffer, was added, and the samples were mixed. The final assay concentrations were: test substance 0.0068-133, respectively 0.00068-13.3 μmol/L, S-2765 0.40 mmol/L (K_{M}=0.25 mmol/L) and FXa 0.1 mmol/L. The linear absorbance increase at 405 nm during 40 min incubation at 37° C. was used for calculation of percent inhibition for the test samples, as compared to references without inhibitor and/ or enzyme. The IC₅₀-value, corresponding to the inhibitor concentration, which caused 50% inhibition of the FXa activity, was calculated by fitting the data to a three-parameter equation by Microsoft XLfit.

b) Measurement of Thrombin Inhibition

The thrombin inhibitor potency was measured with a chromogenic substrate method developed in-house in principle as described in a) for FXa but using instead 0.3 mM of the chromogenic substrate solution S-2366 (Chromogenix, Mölndal, Sweden) and 0.1 nmol/L human thrombin (Haematologic Technologies Inc., Essec Junction, Vt., USA).

c) Measurement of Anticoagulant Activity

An in vitro assay whereby human blood is collected and added directly to a sodium citrate solution (3.2 g/100 mL, 9 parts blood to 1 part citrate solution). Plasma is prepared by centrifugation (1000 g, 15 minutes) and stored at −80° C.) and an aliquot was rapidly thawed at 37° C. on the day of the experiment and kept on ice before addition to the coagulometer cups. Conventional prothrombin time (PT) tests are carried out in the presence of various concentrations of a test compound and the concentration of test compound required to double the clotting time is determined. Thromborel® S (Dade Behring, Liederbach, Germany) was reconstituted with 10 mL water. This solution was kept at 4° C. and was used within one week. Before the experiment the solution was kept at 37° C. for at least 30 minutes before start of the experiment. A ball coagulation timer KC 10A from Heinrich Amelung GmbH. (Lemgo, Germany) was used to study if the compounds could prevent coagulation in human plasma. The time for 50 μl plasma with compound to coagulate after addition of 100 μl Thromborel S, the Prothrombin Time or PTᵢ, is compared with the time it takes for pure plasma to coagulate, PT₀. With this technique the change in viscosity in the stirred solution is used to define clotting. The IC₅₀ is calculated from the curve of PTᵢ/PTₒ versus the inhibitor concentration in plasma, id est three times the final assay concentration.

d) An in vivo Measurement of Antithrombotic Activity

The abdoman is opened and the caval vein exposed. The thrombotic stimulus is partial stasis to the caval vein and a piece of filter paper soaked with ferric chloride and superimposed to the external surface of the vein. Thrombus size is determined as the thrombus wet weight at the end of the experiment. (Ref Thromb. Res. 2002;107:163-168).

When tested in the above mentioned screen a) Measurement of Factor Xa Inhibition, the compounds of the Examples gave IC₅₀ values for inhibition of Factor Xa activity of less than 10 μM, indicating that the compounds of the invention are expected to possess useful therapeutic properties.

Specimen results are shown in the following Table:

| Compound | IC₅₀ value (nM) |
|---|---|
| Example 3 | 4.8 |
| Example 6 | 98 |

A feature of the invention is a compound of formula (I), or a pharmaceutically acceptable salt thereof, for use in medical therapy.

According to a further feature of the invention there is provided a pharmaceutical composition which comprises a compound of formula (I), or a pharmaceutically acceptable salt thereof, in association with a pharmaceutically acceptable diluent or carrier.

The composition may be in a form suitable for oral use, for example a tablet, capsule, aqueous or oily solution, suspension or emulsion; for topical use, for example a cream, ointment, gel or aqueous or oily solution or suspension; for nasal use, for example a snuff, nasal spray or nasal drops; for vaginal or rectal use, for example a suppository; for administration by inhalation, for example as a finely divided powder such as a dry powder, a microcrystalline form or a liquid aerosol; for sub-lingual or buccal use, for example a tablet or capsule; or for parenteral use (including intravenous, subcutaneous, intramuscular, intravascular or infusion), for example a sterile aqueous or oily solution or suspension. In general the above compositions may be prepared in a conventional manner using conventional excipients.

The amount of active ingredient (that is a compound of the formula (I), or a pharmaceutically-acceptable salt thereof) that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient.

According to a farther feature of the invention there is provided a compound of formula (I), or a pharmaceutically-acceptable salt thereof, for use in a method of treatment of the human or animal body by therapy.

The invention also includes the use of such an active ingredient (i.e. a compound of the formula (I), or a pharmaceutically-acceptable salt thereof) in the production of a medicament for use in:
(i) producing a Factor Xa inhibitory effect;
(ii) producing an anticoagulant effect;
(iii) producing an antithrombotic effect;
(iv) treating a Factor Xa mediated disease or medical condition;
(v) treating a thrombosis mediated disease or medical condition;
(vi) treating coagulation disorders; and/or
(vii) treating thrombosis or embolism involving Factor Xa mediated coagulation.

The invention also includes a method of producing an effect as defined hereinbefore or treating a disease or disorder as defined hereinbefore which comprises administering to a warm-blooded animal requiring such treatment an effective amount of an active ingredient as defined hereinbefore.

The size of the dose for therapeutic or prophylactic purposes of a compound of the formula (I) will naturally vary according to the nature and severity of the medical condition, the age and sex of the animal or patient being treated and the route of administration, according to well known principles of medicine. As mentioned above, compounds of the formula (I) are useful in the treatment or prevention of a variety of medical disorders where anticoagulant therapy is indicated. In using a compound of the formula (I) for such a purpose, it will generally be administered so that a daily oral dose in the range, for example, 0.5 to 100 mg/kg body weight/day is received, given if required in divided doses. In general lower doses will be administered when a parenteral route is employed, for example a dose for intravenous administration in the range, for example, 0.01 to 10 mg/kg body weight/day will generally be used. For preferred and especially preferred compounds of the invention, in general, lower doses will be employed, for example a daily dose in the range, for example, 0.1 to 10 mg/kg body weight/day. In general a preferred dose range for either oral or parenteral administration would be 0.01 to 10 mg/kg body weight/day.

Although the compounds of formula (I) are primarily of value as therapeutic or prophylactic agents for use in warm-blooded animals including man, they are also useful whenever it is required to produce an anticoagulant effect, for example during the ex vivo storage of whole blood or in the development of biological tests for compounds having anticoagulant properties.

The compounds of the invention may be administered as a sole therapy or they may be administered in conjunction with other pharmacologically active agents such as a thrombolytic agent, for example tissue plasminogen activator or derivatives thereof or streptokinase. The compounds of the invention may also be administered with, for example, a known platelet aggregation inhibitor (for example aspirin, a thromboxane antagonist or a thromboxane synthase inhibitor), a known hypolipidaemic agent or a known anti hypertensive agent.

The compounds of the invention may also be combined and/or co-administered with any antithrombotic agent(s) with a different mechanism of action, such as one or more of the following: the anticoagulants unfractionated heparin, low molecular weight heparin, other heparin derivatives, synthetic heparin derivatives (e.g. fondaparinux), vitamin K antagonists, synthetic or biotechnological inhibitors of other coagulation factors than FXa (e.g. synthetic thrombin, FVIIa, FXIa and FIXa inhibitors, and rNAPc2), the antiplatelet agents acetylsalicylic acid, ticlopidine and clopidogrel; thromboxane receptor and/or synthetase inhibitors; fibrinogen receptor antagonists; prostacyclin mimetics; phosphodiesterase inhibitors; ADP-receptor (P2X1, P2Y1, P2Y12 [P2T]) antagonists; and inhibitors of carboxypeptidase U (CPU or TAFIa) and inhibitors of plasminogen activator inhibitor-1 (PAI-1).

The compounds of the invention may further be combined and/or co-administered with thrombolytics such as one or more of tissue plasminogen activator (natural, recombinant or modified), streptokinase, urokinase, prourokinase, anisoylated plasminogen-streptokinase activator complex (APSAC), animal salivary gland plasminogen activators, and the like, in the treatment of thrombotic diseases, in particular myocardial infarction.

The invention further relates to a combination comprising a compound of formula (I) and any antithrombotic agent(s) with a different mechanism of action. Said antithrombotic agent(s) may be, for example, one or more of the following: the anticoagulants unfractionated heparin, low molecular weight heparin, other heparin derivatives, synthetic heparin derivatives (e.g. fondaparinux), vitamin K antagonists, synthetic or biotechnological inhibitors of other coagulation factors than FXa (e.g. synthetic thrombin, FVIIa, FXIa and FIXa inhibitors, and rNAPc2), the antiplatelet agents acetylsalicylic acid, ticlopidine and clopidogrel; thromboxane receptor and/or synthetase inhibitors; fibrinogen receptor antagonists; prostacyclin mimetics; phosphodiesterase inhibitors; ADP-receptor (P2X1, P2Y1, P2Y12 [P2T]) antagonists; and inhibitors of carboxypeptidase U (CPU or TAFIa) and inhibitors of plasminogen activator inhibitor-1 (PAI-1).

Moreover, the invention further relates to a combination comprising a compound of formula (I) and thrombolytics, e.g. one or more of tissue plasminogen activator (natural, recombinant or modified), streptokinase, urokinase, prourokinase, anisoylated plasminogen-streptokinase activator complex (APSAC), animal salivary gland plasminogen activators.

Further, the invention also relates to a combination comprising a compound of formula (I) and thrombolytics, e.g. one or more of tissue plasminogen activator (natural, recombinant or modified), streptokinase, urokinase, prourokinase, anisoylated plasminogen-streptokinase activator complex (APSAC), animal salivary gland plasminogen activators, and the like, in the treatment of thrombotic diseases, in particular myocardial infarction.

The invention will now be illustrated in the following Examples in which, unless otherwise stated:

(i) Yields are given for illustration only and are not necessarily the maximum attainable. Single node microwave irradiation was performed using either an Emrys Optimizer or a Smith Creator from Personal Chemistry. All solvents and reagents were la used as purchased without purification unless noted.;

(ii) The end-products have satisfactory high resolution mass spectral (HRMS) data as analysed on a Micromass QT of Micro spectrometer equipped with an Agilent 1100 LC system high performance liquid chromatography (HPLC). The spectrometer was continually calibrated with leucine enkephaline C₂₈H₃₇N₅O₇ (m/z 556.2771). MS conditions: Electrospray ionization, positive mode, capillary voltage 2.3 kV and desolvation temperature 150° C. Accurate mass was determined for positive ionization using leucine enkephaline (m/z 556.2771) as lock mass. Structures were confirmed by ¹H nuclear magnetic resonance (¹H NMR) spectra which were obtained with either a Varian Unity plus or a Varian Inova spectrometer operating at 400, 500 and 600 MHz respectively. Chemical shift values were measured on the delta scale; the following abbreviations have been used: s, singlet; d, doublet; t, triplet; q, quartet; sept, septet; m, multiplet.;

(iii) Isolated intermediates were generally characterised as the end products with the exception of HRMS data.

(iv) Preparative reversed phase HPLC was performed using a Waters Prep LC 2000 with UV detection equipped with a 25 cm×2 cm or 30×5 cm C8 or C18 columns from Kromasil. Preparative chiral resolution using HPLC was performed using a Gilson 306 with UV detection equipped with either a Ciralpak AS (25×2 cm) (ester separations), a Chiralpak AD (25×2 cm) (amide separations) or a Chirobiotic R (25×2 cm) (carboxylic acid separation) column using 100% methanol or methanol/acetic acid/triethyl amine 100/0.1/0.05. All chiral separations were performed at 40° C.

### EXAMPLE 1

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid

The title product of Example 2, i.e. 4-(3-chloro-1H-indole-6-sulfonyl)-1-[1-(1-methyl-6-oxo-1,6-dihydro-pyridazin-3-yl)-piperidin-4-ylmethyl-6-oxo-piperazine-2-carboxylic acid methyl ester, (35 mg, 0.061 mmol) was dissolved in tetrahydrofuran (0.75 mL) and a water solution of lithium hydroxide (1 M, 0.25 mL) was added. The mixture was stirred at room temperature for 1 hour. The reaction mixture was neutralized with acetic acid before purification with HPLC using a gradient of acetonitrile/5% acetonitrile water phase containing 0.1 M ammonium acetate, to give 30 mg (88%) of the title compound.

¹H NMR (500 MHz, dimethyl sulfoxide-d₆ as solvent and internal reference) δ(ppm) 0.88 (dq, 1H, J=4, 12Hz), 1.02 (dq, 1H, J=4, 12Hz), 1.23 (broad d, 1H, J=12 Hz), 1.44 (broad d, 1H, J=12 Hz), 1.52-1.62 (m, 1H), 2.34-2.54 (n, 3H), 2.98 (dd, 1H, J=4.4, 11.3 Hz), 3.35 (d, 1H, J=16.1 Hz), 3.57-3.70 (m, 5H), 3.77 (dd, 1H, J=3.8, 11.3 Hz), 6.75 (d, 1H, J=10.0 Hz), 7.38 (d, 1H, J=10.0 Hz), 7.46 (dd, 1H, J=1.6, 8.4 Hz), 7.70 (d, 1H, J=8.4 Hz), 7.85-7.87 (m, 2H).

HRMS (ESI+) calc. [M+H]⁺ 563.1474, found 563.1489.

### EXAMPLE 2

### (R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid methyl ester

### A) (R)-4-(1-Benzenesulfonyl-3-chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid methyl ester

To a mixture of (R)-1-[1-(1-methyl-6-oxo-1,6-dihydro-pyridazin-3-yl)-piperidin-4-ylmethyl]-6-oxo-piperazine-2-carboxylic acid methyl ester hydrochloride (185 mg, 0.46 mmol) in anhydrous dichloromethane/N,N-dimethylformamide 5:1 (4 mL) was added pyridine (0.10 mL, 1.2 mmol) at 0° C. under nitrogen atmosphere. To the mixture, a solution of 1-benzenesulfonyl-3-chloro-1H-indole-6-sulfonyl chloride (181 mg, 0.46 mmol) in anhydrous dichloromethane (2 mL) was added at 0° C., and the reaction mixture was stirred at room temperature for 20 minutes. The solvent was removed in vacuo before purification with HPLC using a gradient of acetonitrile/5% acetonitrile-water phase containing 0.1 M ammonium acetate, to give 150 mg (45%) of the sub-title compound after evaporation and freeze drying over night. The sub-title compound was used directly in step B.

B)

The title compound was synthesized and purified essentially as in example 4, step E using the product from step A, i.e. (R)-4-(1-benzenesulfonyl-3-chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid methyl ester, (150 mg, 0.21 mmol) as starting material to give 62 mg (51%).

¹H NMR (500 MHz, dimethyl sulfoxide-d₆ as solvent and internal reference) δ(ppm) 0.99 (dq, 1H, J=4, 12 Hz), 1.11 (dq, 1H, J=4, 12 Hz), 1.45 (broad d, 1H, J=12 Hz), 1.56 (broad d, 1H, J=12 Hz), 1.64-1.74 (m, 1H), 2.48-2.64 (m, 3H), 3.01 (dd, 1H J=3.4, 12.2 Hz), 3.33-3.35 (m, 1H), 3.44 (s, 3H), 3.68 (s, 3H), 3.68-3.77 (m, 3H), 3.81 (d, 1H, J=16.1 Hz), 3.99 (d, 1H, J=12.2 Hz), 4.41 (t, 1H, J=2.7 Hz), 6.75 (d, 1H, J=10.0 Hz), 7.41 (d, 1H, J=10.0 Hz), 7.47 (dd, 1H, J=1.6, 8.4 Hz), 7.72 (d, 1H, J=8.4 Hz), 7.87 (d, 1H, J=1.2 Hz), 7.88 (s, 1H).

HRMS (ESI+) calc. [M+H]⁺ 577.1630, found 577.1622.

### EXAMPLE 3

### 6-{4-[4-(3-Chloro-1H-indole-6-sulfonyl)-3-hydroxy-piperazine-1-carbonyl]-piperidin-1-yl}-2-methyl-2H-pyridazin-3-one

### A) 1-(1-Methyl-6-oxo-1,6-dihydro-pyridazin-3-yl)-piperidine-4-carboxylic acid

Triethylamine (2.8 mL, 20 mmol) was added to a mixture of 6-chloro-2-methyl-2H-pyridazin-3-one (578 mg, 4.00 mmol) and piperidine-4-carboxylic acid (775 mg, 6.00 mmol) in 6.5 mL ethanol/water 3:1 in a microwave vial and heated at 180° C. for 15 hours. After cooling to room temperature 2 M sodium hydroxide (4 mL) was added to the reaction mixture. Ethanol and triethylamine were removed in vacuo and the basic aqueous solution was heated at 70° C. for 1.5 hours, diluted to 50 mL and washed twice with 20 mL ethyl acetate. The pH was adjusted to 5 using aqueous hydrochloric acid (a precipitate formed) and the volume of the mixture was reduced to 20 mL. The mixture was placed in the refrigerator over night and the solids were collected by filtration, washed with a small amount of water and dried under vacuum to give 497 mg of the sub-title compound (52%).

¹H NMR (400 Hz, dimethyl sulphoxide-d₆ as solvent and internal reference) δ(ppm) 1.54 (m, 2H), 1.84 (m, 2H), 2.40 (m, 1H), 2.76 (m, 2H), 3.47 (s, 3H), 3.74 (m, 2H), 6.78 (d, 1H, J=9.6 Hz), 7.47 (d, 1H, J=9.6 Hz).

### B) (2-{Allyl-[1-(1-methyl-6-oxo-1,6-dihydro-pyridazin-3-yl)-piperidine-4-carbonyl]-amino}-ethyl)-carbamic acid tert-butyl ester

1-Ethyl-3-(3-dimethylaminopropyl)carbodiimide hydrochloride (1.58 g, 8.22 mmol) was added to a stirred suspension of 1-(1-methyl-6-oxo-1,6-dihydro-pyridazin-3-yl)-piperidine-4-carboxylic acid (976 mg, 4.11 mmol), (2-allylamino-ethyl)-carbamic acid tert-butyl ester (1.18 g, 5.30 mmol) and 4-dimethylaminopyridine (2.01 g, 16.5 mmol) in anhydrous N,N-dimethylformamide (16 mL) at room temperature and the resulting suspension was stirred overnight. The resulting slightly cloudy solution was poured into a mixture of ice and water and the pH was adjusted to 4 using 1 M aqueous potassium hydrogensulfate while maintaining the temperature at 0° C. The aqueous solution was extracted with three portions of dichloromethane and the combined organic layers were washed with brine, dried, filtered, concentrated and pumped under high-vacuum to give the crude sub-title compound (1.93 g, 95% yield) as an oil which was used without further purification.

¹H NMR (500 MHz; chloroform-d as solvent and internal reference, major rotamer reported) δ(ppm) 7.10 (broad d, 1H, J=9.9 Hz), 6.83 (d, 1H, J=9.8 Hz), 5.72-5.86 (m, 1H), 5.24 (broad d, 1H, J=10.5 Hz), 5.10-5.18 (m, 1H), 4.95-5.01 (m, 1H), 3.96-4.00 (m, 2H), 3.81-3.87 (m, 2H), 3.63 (s, 3H), 3.41-3.50 (m, 2H), 3.21-3.30 (m, 2H), 2.70-2.84 (m, 2 H), 2.53-2.61 (m, 1H), 1.84-1.96 (m, 2H), 1.69-1.82 (m, 2H), 1.43 (s,9H)

### C) 1-(1-Methyl-6-oxo-1,6-dihydro-pyridazin-3-yl)-piperidine-4-carboxylic acid allyl-(2-amino-ethyl)-amide dihydrochloride

Crude (2-{allyl-[1-(1-methyl-6-oxo-1,6-dihydro-pyridazin-3-yl)-piperidine-4-carbonyl]-amino}-ethyl)-carbamic acid tert-butyl ester from step B (1.9 g, 3.9 mmol) was dissolved in 99.5% ethanol (20 mL) and cooled to 0° C. A 4 M solution of hydrochloric acid in dioxane (40 mL) was added dropwise and the reaction was stirred at 0° C. for 5 minutes and then for 1.5 hours at room temperature. The solvents were removed in vacuo and the residue was pumped under high-vacuum at 30° C. to give the crude sub-title compound (1.78 g, contains residual solvents, quantitative yield) as a foam which was used without further purification.

¹H NMR (500 MHz; methanol-d₄ as solvent and internal reference, major rotamer reported) δ(ppm) 7.69 (d, 1H, J=9.9 Hz), 7.10 (d, 1H, J=9.9 Hz), 5.90-5.98 (m, 1H), 5.29 (d, 1H, J=10.5 Hz), 5.22 (d, 1H, J=17.3), 4.90 (broad d, 1H, J=4.9 Hz), 4.06-4.12 (m, 2H), 3.77 (s, 3H), 3.60 (t, 2H, J=6.2 Hz), 3.10 (t, 2H, J=6.2 Hz), 2.85-2.98 (m, 3H), 1.73-1.86 (m, 4H).

### D) 1-(1-Methyl-6-oxo-1,6-dihydro-pyridazin-3-yl)-piperidine-4-carboxylic acid allyl-[2-(1-benzenesulfonyl-3-chloro-1H-indole-6-sulfonylamino)-ethyl]-amide

A suspension of crude 1-(1-methyl-6-oxo-1,6-dihydro-pyridazin-3-yl)-piperidine-4-carboxylic acid allyl-(2-amino-ethyl)-amide dihydrochloride (224 mg, 0.457 mmol) from step C in anhydrous dichloromethane (3 mL) was added to a stirred solution of 1-benzenesulfonyl-3-chloro-1H-indole-6-sulfonyl chloride (140 mg, 0.370 mmol) and N,N-diisopropylethylamine (0.26 mL, 1.48 mmol) in anhydrous dichloromethane (1 mL) at 0° C. The reaction mixture was stirred at room temperature for 3 hours and then diluted with dichloromethane. Water was added and the aqueous layer was titrated to pH 4 using 1 M aqueous potassium hydrogensulfate and saturated aqueous sodium hydrogen carbonate. The layers were mixed thoroughly and then separated. The aqueous layer was extracted with a second portion of dichloromethane. The combined organic layers were washed with brine, dried, filtered and concentrated. The residue was purified by flash chromatography on silica gel eluted with 50:1 dichloromethane/methanol to give the sub-title compound (220 mg, 88.3%).

¹H NMR (500 MHz; chloroform-d as solvent and internal reference, major rotamer reported) δ(ppm) 8.52 (d, 1H, J=1.4 Hz), 7.91-7.93 (m, 2H), 7.98 (dd, 1H, J=8.21, 1.5 Hz), 7.72 (s, 1H), 7.65 (d, 1H, J=8.4 Hz), 7.58-7.62 (m, 1H), 7.48-7.52 (m, 2H), 7.10 (d, 1H, J=9.9 Hz), 6.84 (d, 1H, J=9.9 Hz), 5.89 (t, 1H, 5.2 Hz), 5.77 ddt, 1H, J=17.2, 10.4, 4.8 Hz), 5.22 (broad d, 1H, J=10.2 Hz), 5.13 (broad d, 1H, J=17.2 Hz), 3.98-4.01 (m, 2H), 3.81-3.86 (m, 2H), 3.64 (s, 3H), 3.48 (t, 2H, J=5.8 Hz), 3.14 (q, 2H, J=5.6 Hz), 2.73 (td, 2H, J=12.7, 2.6 Hz), 2.55 (tt, 1H, J=11.3, 3.7 Hz), 1.68-1.92 (m, 4H).

### E) 1-(1-Methyl-6-oxo-1,6-dihydro-pyridazin-3-yl)-piperidine-4-carboxylic acid allyl-[2-(3-chloro-1H-indole-6-sulfonylamino)-ethyl]-amide

1-(1-Methyl-6-oxo-1,6-dihydro-pyridazin-3-yl)-piperidine-4-carboxylic acid allyl-[2-(1-benzenesulfonyl-3-chloro-1H-indole-6-sulfonylamino)-ethyl]-amide (220 mg, 0.33 mmol) from step D was treated essentially as in example 4, step E to give the sub-title compound (73 mg, 42% yield) as a solid.

¹H NMR (500 MHz; chloroform-d as solvent and internal reference, major rotamer reported) δ(ppm) 9.58 (broad s, 1H), 7.98 (broad s, 1H), 7.71 (d, 1H, J=8.5 Hz), 7.60 (dd, 1H, J=8.5, 1.4 Hz), 7.38-7.40 (m, 1H), 7.08 (d, 1H, J=9.9 Hz), 6.84 (d, 1H, J=9.9 Hz), 5.62-5.79 (m, 2H), 5.21 (d, 1H, J=10.2 Hz), 5.11 (d, 1H, J=17.2 Hz), 3.93-3.98 (m, 2H), 3.77-3.83 (m, 2H), 3.64 (s, 3H), 3.46 (t, 2H, J=5.7 Hz), 3.06-3.18 (m, 2H), 2.71 (td, 2H, J=12.8, 2.34 Hz), 2.53 (tt, 1H, J=11.3 Hz, 3.6 Hz), 1.65-1.92 (m, 4H).

### F)

1-(1-Methyl-6-oxo-1,6-dihydro-pyridazin-3-yl)-piperidine-4-carboxylic acid allyl-[2-(3-chloro-1H-indole-6-sulfonylamino)-ethyl]-amide (69 mg, 0.13 mmol) from step E was treated essentially as in example 4, step F to give the title compound (38 mg, 55% yield) as a solid.

¹H NMR (500 MHz; acetonitrile-d₃ as solvent and internal reference, two rotamers) δ(ppm) 9.99 (broad s, 1H), 7.99 (broad s, 1H), 7.67 (broad d, 1H, J=8.4 Hz), 7.57 (broad d, 1H, J=8.4 Hz), 7.52 (s, 1H), 7.20 (d, 1H, J=9.9 Hz), 6.69 (d, 1H, J=9.9 Hz), 5.49 (broad s, 1H), 4.33-4.45 (m, 1H), 3.93 (broad d, 1H, J=13.4 Hz), 3.72-3.80 (m, 2H), 3.49 (s, 3H), 3.42-3.53 (m, 1H), 3.29 (broad d, 0.6 H, J=13.2 Hz, major rotamer), 3.17 (broad t, 0.4 H, J=11.9 Hz, minor rotamer), 3.07 ( broad t, 0.4 H, J=11.9 Hz, minor rotamer), 2.95 (broad t, 0.6 H, J=11.3 Hz, major rotamer), 2.85 (broad d, 0.4 H, J=13.2 Hz), 2.68-2.80 (m, 4H), 1.55-1.80 (m, 4H).

HRMS (ESI+) calc. [M+H]⁺ 535.1525, found 535.1525.

### EXAMPLE 4

### 6-{4-[4-(3-Chloro-1H-indole-6-sulfonyl)-2-hydroxy-piperazine-1-carbonyl]-piperidin-1-yl}-2-methyl-2H-pyridazin-3-one

### A) (2-{[1-(1-Methyl-6-oxo-1,6-dihydro-pyridazin-3-yl)-piperidine-4-carbonyl]-amino}-ethyl)-carbamic acid tert-butyl ester

1-Ethyl-3-(3-dimethylaminopropyl)carbodiimide hydrochloride (695 mg, 3.62 mmol) was added to a stirred solution of 1-(1-methyl-6-oxo-1,6-dihydro-pyridazin-3-yl)-piperidine-4-carboxylic acid (430 mg, 1.81 mmol), N-boc-ethylenediamine (348 mg, 2.17 mmol) and 4-dimethylaminopyridine (886 mg, 3.62 mmol) in anhydrous N,N-dimethylformamide (8 mL) at room temperature and the solution was stirred overnight. The reaction mixture was poured onto ice-water and the pH was adjusted to pH 6 using 1 M aqueous potassium hydrogensulfate and the aqueous solution was extracted twice with ethyl acetate. The combined organic layers were washed with saturated aqueous sodium bicarbonate solution followed by brine, dried, filtered and concentrated to give crude (2-{[1-(1-methyl-6-oxo-1,6-dihydro-pyridazin-3-yl)-piperidine-4-carbonyl]-amino}-ethyl)-carbamic acid tert-butyl ester (400 mg). Further extraction of the aqueous reaction mixture with five portions of ethyl acetate essentially as described above gave an additional 180 mg of the crude sub-title compound to give a total of 580 mg (1.53 mmol, 84% yield) which was used without further purification.

¹H NMR (400 MHz; chloroform-d as solvent and internal reference) δ(ppm) 7.09 (d, 1H, J=10.1 Hz), 6.83 (d, 1H, J=10.1 Hz), 6.40-6.51 (m, 1H), 4.81-4.98 (m, 1H), 3.78-3.86 (m, 2H), 3.64 (s, 3H), 3.32-3.38 (m, 2H), 3.25-2.32 (m, 2H), 2.72-2.80 (m, 2H), 2.26 (tt, 1H, J=11.5, 3.8 Hz), 1.88-1.95 (m, 2H), 1.71-1.83 (m, 2H), 1.43 (s, 9H).

### B) 1-(1-Methyl-6-oxo-1,6-dihydro-pyridazin-3-yl)-piperidine-4-carboxylic acid (2-amino-ethyl)-amide hydrochloride

(2-{[1-(1-Methyl-6-oxo-1,6-dihydro-pyridazin-3-yl)-piperidine-4-carbonyl]-amino}-ethyl)-carbamic acid tert-butyl ester (580 mg, 1.52 mmol) from step A was suspended in 99.5% ethanol (5 mL) and cooled by an ice-bath. Hydrogen chloride (4 M solution in dioxane, 10 mL) was added dropwise and the reaction mixture was stirred at 0° C. for 30 minutes followed by 1 hour at room temperature. The solvents were removed in vacuo and the residue was dissolved in water and freeze-dried to give the crude sub-title compound (0.54 g, quantitative yield) as a solid which was used without further purification.

¹H NMR (400 MHz; dimethyl sulfoxide-d₆ as solvent and internal reference) δ(ppm) 8.19 (t, 1H, J=5.6 Hz), 8.11 (broad s, 3H), 7.51 (d, 1H, J=10.0 Hz), 6.81 (d, 1H, 10.0 Hz), 3.82-3.89 (m, 2H), 3.49 (s, 3H), 3.30 (q, 2H, J=6.1 Hz), 2.80-2.88 (m, 2H), 2.70 (dt, 2H, J=12.6, 2.4 Hz), 2.32 (tt, 1H, J=11.6, 3.9 Hz), 1.75-1.82 (m, 2H), 1.52-1.63 (m, 2H).

### C) 1-(1-Methyl-6-oxo-1,6-dihydro-pyridazin-3-yl)-piperidine-4-carboxylic acid [2-(1-benzenesulfonyl-3-chloro-1H-indole-6-sulfonylamino)-ethyl]-amide

A solution of 1-benzenesulfonyl-3-chloro-1H-indole-6-sulfonyl chloride (178 mg, 0.46 mmol) in anhydrous dichloromethane (2.5 mL) was added to a mixture of crude 1-(1-methyl-6-oxo-1,6-dihydro-pyridazin-3-yl)-piperidine-4-carboxylic acid (2-amino-ethyl)-amide hydrochloride (241 mg, 0.68 mmol) from step B and diisopropylethylamine (235 mg, 1.82 mmol) in anhydrous dichloromethane (1 mL) and the reaction was stirred at room temperature overnight. Dichloromethane and water was added and the aqueous layer was titrated to pH 4 using 1 M aqueous potassium hydrogensulfate and the layers were separated. The aqueous layer was extracted with two portions of dichloromethane and the combined organic layers were washed with brine, dried over magnesium sulfate, filtered and concentrated. The crude was purified by flash chromatography using a gradient of methanol in dichloromethane to give the sub-title compound (150 mg, 52% yield).

¹H NMR (400 MHz; chloroform-d as solvent and internal reference) δ(ppm) 8.54 (dd, 1H, J=1.6, 0.7 Hz), 7.92-7.95 (m, 2H), 7.79 (dd, 1H, J=8.3, 1.5 Hz), 7.74 (s, 1H) 7.69 (dd, 1H, J=8.3, 0.5 Hz), 7.60-7.65 (m, 1H), 7.50-7.55 (m, 2H), 7.11 (d, 1H, J=9.9 Hz), 6.85 (d, 1H, J=9.9 Hz), 6.11 (t, 1H, J=5.8 Hz), 5.36 (t, 1H, J=6.0 Hz), 3.81-3.87 (m, 2H), 3.65 (s, 3H), 3.48-3.43 (m, 2H), 3.08-3.13 (m, 2H), 2.72-2.80 (m, 2H), 2.27 (tt, 1H, J=11.6, 4.0 Hz), 1.86-1.94 (m, 2H), 1.71-1.83 (m, 2H).

### D) 1-(1-Methyl-6-oxo-1,6-dihydro-pyridazin-3-yl)-piperidine-4-carboxylic acid {2-[allyl-(1-benzenesulfonyl-3-chloro-1H-indole-6-sulfonyl)-amino]-ethyl}-amide

A mixture of crude 1-(1-methyl-6-oxo-1,6-dihydro-pyridazin-3-yl)-piperidine-4-carboxylic acid [2-(1-benzenesulfonyl-3-chloro-1H-indole-6-sulfonylamino)-ethyl]-amide (123 mg, 0.19 mmol) from previous step, allyl bromide (70 mg, 0.58 mmol) and potassium carbonate (115 mg, 0.835 mmol) in anhydrous acetonitrile was stirred overnight at room temperature. An additional 70 mg (0.58 mmol) of allyl bromide and 80 mg (0.58 mmol) of potassium carbonate were added in three portions and the reaction was again stirred overnight. The reaction mixture was diluted with dichloromethane and washed with water. The aqueous layer was extracted with dichloromethane and the combined organic layers were washed with brine, dried, filtered and concentrated to give the crude sub-title compound (0.14 g, quantitative yield) which was used without further purification.

¹H NMR (400 MHz; chloroform-d as solvent and internal reference) δ(ppm) 8.48 (dd, 1H, J=1.5, 0.8 Hz), 7.90-7.93 (m, 2H), 7.76 (s, 1H), 7.73 (AB dd, 1H, J=8.4, 1.4 Hz), 7.70 (AB dd, 1H, J=8.4, 0.6 Hz), 7.60 (m, 1H), 7.49-7.54 (m, 2H), 7.12 (d, 1H, J=9.9 Hz), 6.84 (d, 1H, J=9.9 Hz), 6.26 (broad t, 1H, J=5.1 Hz), 5.50-5.60 (m, 1H), 5.13-5.21 (m, 2H), 3.84-3.91 (m, 4H), 3.65 (s, 3H), 3.41-3.46 (m, 2H), 3.23-3.28 (m, 2H), 2.76-2.84 (m, 2H), 2.34 (tt, 1H, J=11.7, 3.8 Hz), 1.96-2.03 (m, 2H), 1.78-1.90 (m, 2H).

### E) 1-(1-Methyl-6-oxo-1,6-dihydro-pyridazin-3-yl)-piperidine-4-carboxylic acid {2-[allyl-(3-chloro-1H-indole-6-sulfonyl)-amino]-ethyl}-amide

Crude 1-(1-methyl-6-oxo-1,6-dihydro-pyridazin-3-yl)-piperidine-4-carboxylic acid {2-[allyl-(1-benzenesulfonyl-3-chloro-1H-indole-6-sulfonyl)-amino]-ethyl}-amide (133 mg, 0.20 mmol) from step D was dissolved in anhydrous tetrahydrofuran and a 1 M solution of tetrabutylammonium fluoride (0.20 mL, 0.2 mmol) in tetrahydrofuran was added. The reaction was heated by single node microwave irradiation at 100° C. for 8 minutes. A second portion of 1 M tetrabutylammonium fluoride (0.025 mL, 0.025 mmol) in tetrahydrofuran was added and the reaction was heated for an additional 3 minutes at 100° C. The solvent was removed in vacuo and the crude was purified by preparative HPLC using a gradient of acetonitrile/5% acetonitrile-water phase containing 0.1 M ammonium acetate, to give the sub-title compound (65 mg, 62% yield) as a solid.

¹H NMR (400 MHz; chloroform-d as solvent and internal reference) δ(ppm) 8.67 (broad s, 1H), 7.91 (dd, 1H, J=1.5, 0.5 Hz), 7.76 (d, 1H, J=8.4 Hz), 7.57 (dd, 1H, J=8.46. 1.6 Hz), 7.42-7.43 (m, 1H), 7.12 (d, 1H, J=9.9 Hz), 6.85 (d, 1H, J=9.9 Hz), 6.35 (broad t, 1H, J=4.9 Hz), 5.59 (ddt, 1H, J=17.2, 10.2, 6.6 Hz), 5.12-5.20 (m, 2H), 3.84-3.90 (m, 4H), 3.66 (s, 3H), 3.39-3.44 (m, 2H), 3.26-3.29 (m, 2H), 2.76-2.84 (m, 2H), 2.33 (tt, 1H, J=11.7, 3.8 Hz), 1.94-2.01 (m, 2H), 1.77-1.88 (m, 2H).

### F)

A solution of sodium periodate (77 mg, 0.36 mmol) in water (0.5 mL) was added to a stirred solution of 1-(1-methyl-6-oxo-1,6-dihydro-pyridazin-3-yl)-piperidine-4-carboxylic acid {2-[allyl-(3-chloro-1H-indole-6-sulfonyl)-amino]-ethyl}-amide (60 mg, 0.11 mmol) from step E in tetrahydrofuran (1.5 mL). Osmium tetroxide (0.030 mL of a 2.5% wt solution in tert-butanol, 0.0030 mmol) was added and the reaction was stirred overnight during which a precipitate formed. The reaction mixture was diluted with dichloromethane and washed with water. The aqueous layer was extracted with ethyl acetate and the two organic layers were each washed with brine, combined, dried, filtered and concentrated. The crude was purified twice by preparative HPLC using a gradient of acetonitrile/5% acetonitrile-water phase containing 0.1 M ammonium acetate, to give the title compound (13.7 mg, 22.7% yield) as a solid.

¹H NMR (400 MHz; acetonitrile-d₃ as solvent and internal reference, two rotamers in 3:2 ratio) δ(ppm) 7.89 dd, 1H, J=1.4, 0.5 Hz), 7.70 (dd, 1H, J=8.4, 0.5 Hz), 7.54.(s, 1H), 7.45 (dd, 1H, J=8.4, 1.6 Hz), 7.30 (d, 1H, J=10.0 Hz), 6.79 (d, 1H, J=10.0 Hz), 5.87 (broad s, 0.4 H, minor rotamer), 5.54 (broad s, 0.6 H, major rotamer), 4.10-4.18 (broad d, 0.4 H, 13.4 Hz), 3.66-3.86 (m, 4H), 3.51 (s, 3H), 3.46-3.61 (m, 1H), 3.10 (broad t, 1H, J=12.8 Hz), 2.57-2.79 (m, 3H), 2.20-2.51 (m, 2H), 1.40-1.75 (m, 4H).

HRMS (ESI+) calc. [M+H]⁺ 535.1525, found 535.1509.

### EXAMPLE 5

### 6-{4-[4-(3-Chloro-1H-indole-6-sulfonyl)-2-oxo-piperazin-1-ylmethyl]-piperidin-1-yl}-2-methyl-2H-pyridazin-3-one

### A) 1-(1-Methyl-6-oxo-1,6-dihydro-pyridazin-3-yl)-piperidine-4-carbaldehyde

To a solution of oxalyl chloride (5 mL 2.0 M solution in dichloromethane, 10 mmol) in anhydrous dichloromethane (35 mL) was added a solution of dimethyl sulfoxide (1.6 mL) in anhydrous dichloromethane (35 mL) at −78° C. dropwise under argon. During addition, the reaction temperature was kept below −65° C. The reaction mixture was stirred at −73° C. for 1 hour, whereupon a solution of 6-(4-hydroxymethyl-piperidin-1-yl)-2-methyl-2H-pyridazin-3-one (1.73 g, 7.74 mmol) in anhydrous dimethyl sulfoxide (20 mL) and anhydrous dichloromethane (20 mL) were added dropwise. The reaction mixture was stirred at between −70° C. and −65° C. for 1.5 hours then cooled to −73° C. and triethylamine (4.1 mL) was added dropwise. The reaction mixture was allowed to attain room temperature, water and dichloromethane were added. The organic phase was separated, and the aqueous phase was extracted twice with dichloromethane. The combined organic phases were washed with water, brine, dried and evaporated to dryness to give 1.7 (98%) of the sub-title compound.

¹H NMR (500 MHz, chloroform-d as solvent and internal reference) δ (ppm) 1.65 (dq, 2H, J=3.9, 13.8 Hz), 1.95 (dd, 2H, J=3.4, 13.5 Hz), 2.35-2.45 (m, 1H), 2.82-2.91 (m, 2H), 3.58 (s, 3H), 3.63-3.70 (m, 2H), 6.78 (d, 1H, J=9.9 Hz), 7.07 (d, 1H, J=9.9 Hz), 9.62 (s, 1H).

### B) (2-{[1-(1-Methyl-6-oxo-1,6-dihydro-pyridazin-3-yl)-piperidin-4-ylmethyl]-amino}-ethyl)-carbamic acid tert-butyl ester

To a solution of 1-(1-methyl-6-oxo-1,6-dihydro-pyridazin-3-yl)-piperidine-4-carbaldehyde (0.85 g, 3.4 mmol) from step A in anhydrous dichloromethane (16 mL) was added a solution of N-(tert-butoxycarbonyl)-1,2-diaminoethane (0.62 g, 3.8 mmol) in anhydrous dichloromethane (4 mL) and acetic acid (0.46 mL, 8.06 mmol) under argon. After stirring the resulting mixture at room temperature for 1 hour, sodium triacetoxy borohydride (2.85 g, 13.4 mmol) was added and the mixture was stirred over night. Water and dichloromethane were added, and then the aqueous phase was separated and freeze dried over night. The residue was suspended in dichloromethane, filtered and the solution was evaporated to dryness. The crude product was purified by column chromatography on silica gel using dichloromethane/methanol (100:10 and 100:15) as eluent to give 0.54 g (39%) of the sub-title compound.

¹H NMR (300 MHz, methanol-d₄ as solvent and internal reference) δ (ppm) 1.25-1.40 (m, 2H), 1.45 (s, 9H), 1.71-1.95 (m, 3H), 2.70-3.05 (m, 6H), 3.20-3.35 (m, 2H), 3.63 (s, 3H), 3.88-4.06 (m, 2H), 6.85 (d, 1H, J=9.90 Hz), 7.47 (d, 1H, J=9.90 Hz).

### C) (2-{(2-Chloro-acetyl)-[1-(1-methyl-6-oxo-1,6-dihydro-pyridazin-3-yl)-piperidin-4-ylmethyl]-amino}-ethyl)-carbamic acid tert-butyl ester

To a solution of (2-{[1-(1-methyl-6-oxo-1,6-dihydro-pyridazin-3-yl)-piperidin-4-ylmethyl]-amino}-ethyl)-carbamic acid tert-butyl ester (0.41 g, 1.12 mmol) from step B in anhydrous dichloromethane (12 mL) was added triethylamine (0.47 mL, 3.37 mmol) at 0° C. under argon. A solution of bromoacetyl chloride (0.27 g, 1.68 mmol) in anhydrous dichloromethane (2 mL) was added at 0° C. to the mixture dropwise, and then the reaction mixture was stirred at room temperature for 75 minutes. The reaction flask was cooled to 0° C., and water/dichloromethane was added. The organic phase was separated, washed with brine, dried and evaporated under reduced pressure. The crude product was purified by column chromatography on silica gel using dichloromethane/methanol (100:5) as eluent to give 0.22 g (44%) of the sub-title compound.

¹H NMR (300 MHz, methanol-d₄ as solvent and internal reference) δ (ppm) 1.25-1.40 (m, 2H), 1.42 (s, 9H), 1.60-2.05 (m, 3H), 2.65-2.85 (m, 2H), 3.17-3.38 (m, 4H), 3.42-3.54 (m, 2H), 3.62 (s, 3H), 3.85-4.10 (m, 3H), 3.28 (d, 1H, J=4.4 Hz), 6.85 (d, 1H, J=9.9 Hz), 7.45 (d, 1H, J=9.9 Hz).

### D) N-(2-Amino-ethyl)-2-chloro-N-[1-(1-methyl-6-oxo-1,6-dihydro-pyridazin-3-yl)-piperidin-4-ylmethyl]-acetamide hydrochloride

To a solution of (2-{(2-chloro-acetyl)-[1-(1-methyl-6-oxo-1,6-dihydro-pyridazin-3-yl)-piperidin-4-ylmethyl]-amino}-ethyl)-carbamic acid tert-butyl ester (0.22 g, 0.5 mmol) from step C in methanol (10 mL) was added a saturated methanolic hydrochloric acid (10 mL) at 0° C. After stirring at room temperature for 40 minutes, the solution was evaporated to dryness. The residue was dissolved in methanol and the solution evaporated to dryness to give 0.19 (98%) of the sub-title compound. The product was used directly in the next step.

### E) 2-Methyl-6-[4-(2-oxo-piperazin-1-ylmethyl)-piperidin-1-yl]-2H-pyridazin-3-one

To a solution of N-(2-amino-ethyl)-2-chloro-N-[1-(1-methyl-6-oxo-1,6-dihydro-pyridazin-3-yl)-piperidin-4-ylmethyl]-acetamide hydrochloride (0.19 g, 0.48 mmol) from step D in anhydrous N,N-dimethylformamide (3.5 mL) was added triethylamine (0.5 mL) at 0° C. under nitrogen. After stirring at room temperature for 2.5 hours, the solution evaporated to dryness and the crude product was purified by preparative HPLC using acetonitrile and ammonium acetate buffer (5:95 to 40:60) as eluent to give 90 mg (55%) of the sub-title compound.

¹H NMR (500 MHz, methanol-d₄ as solvent and internal reference) δ (ppm) 1.33 (dq, 2H, J=3.6, 12.3 Hz), 1.76 (d, 2H, J=12.7 Hz), 1.95-1.99 (m, 1H), 2.75-2.83 (m, 2H), 3.11 (t, 2H, J=5.2 Hz), 3.30-3.38 (m, 2H), 3.44 (t, 2H, J=5.2 Hz), 3.48 (s, 2H), 3.65 (s, 3H), 3.97 (d, 2H, J=13.0 Hz), 6.87 (d, 1H, J=9.9 Hz), 7.49 (d, 1H, J=10.1 Hz).

### F) 6-{4-[4-(1-Benzenesulfonyl-3-chloro-1H-indole-6-sulfonyl)-2-oxo-piperazin-1-ylmethyl]-piperidin-1-yl}-2-methyl-2H-pyridazin-3-one

To a solution of 2-methyl-6-[4-(2-oxo-piperazin-1-ylmethyl)-piperidin-1-yl]-2H-pyridazin-3-one (90 mg, 0.30 mmol) from step E in anhydrous N,N-dimethylformamide (2 mL) was added triethylamine (0.12 mL, 0.89 mmol) at 0° C. under nitrogen. To the mixture, a solution of 1-benzenesulfonyl-3-chloro-1H-indole-6-sulfonyl chloride (115 mg, 0.30 mmol) in anhydrous dichloromethane (2 mL) was added at 0° C., and the reaction mixture was stirred at room temperature for 1 hour. The reaction flask was cooled to 0° C., and water/dichloromethane was added. The organic phase was separated, washed with brine, dried and evaporated to dryness. The residue was suspended in ethanol, and the solids formed were filtered, washed with ethanol and dried in vacuo to give 130 mg (67%) of the sub-title compound.

### G)

A mixture of 6-{4-[4-(1-benzenesulfonyl-3-chloro-1H-indole-6-sulfonyl)-2-oxo-piperazin-1-ylmethyl]-piperidin-1-yl}-2-methyl-2H-pyridazin-3-one (0.13 g, 0.19 mmol) from step F and tetrabutylammonium fluoride (0.31 mL 1.0 M solution in tetrahydrofuran, 0.31 mmol) in tetrahydrofuran (2 mL) and ethanol (2 mL) was heated in a microwave oven at 100° C. for 12 minutes. The solution was concentrated in vacuo, and the residue was triturated with water to remove tetrabutylammonium fluoride. The crude product was purified by column chromatography on silica gel using dichloromethane/methanol (100:5) as eluent to give 72 mg (71%) of the title compound.

¹H NMR (500 MHz, methanol-d₄ as solvent and internal reference) δ (ppm) 1.10 (dq, 2H, J=3.8, 12.4 Hz), 1.39 (d, 2H, J=12.4 Hz), 1.65-1.74 (m, 1H), 2.55 (t, 2H, J=12.3 Hz), 3.19 (d, 2H, J=7.6 Hz), 3.35 (t, 2H, J=5.9 Hz), 3.44 (t, 2H, J=5.9 Hz), 3.61 (s, 3H), 3.74-3.80 (m, 4H), 6.82 (d, 1H, J=10.0 Hz), 7.38 (d, 1H, J=9.9 Hz), 7.56 (dd, 1H, J=1.5, 8.5 Hz), 7.59 (s, 1H), 7.74 (d, 1H, J=8.5 Hz), 7.95 (d, 1H, J=1.2 Hz).

HRMS (ESI+) calc. [M+H]⁺ 519.1576, found 519.1556.

### EXAMPLE 6

### 4-[4-(3-Chloro-1H-indole-6-sulfonyl)-piperazine-1-carbonyl]-5′-methyl-3,4,5,6-tetrahydro-2H,1′H-[1,3′]bipyridinyl-6′-one

### A) 5-bromo-2-methoxy-3-methyl-pyridine

A suspension of 2,5-dibromo-3-methylpyridine (2.08 g, 8.3 mmol) in a 2 M solution of sodium methoxide in methanol (17 mL) was heated by single node microwave irradiation at 120° C. for 40 minutes. The reaction mixture was poured onto a mixture of ice and 1 M aqueous hydrochloric acid and extracted with two portions of dichloromethane. The combined organic layers were dried, filtered and concentrated in vacuo to give 1.57 g (89%) of the sub-title compound which was used without further purification.

¹H NMR (400 MHz; chloroform-d as solvent and internal reference) δ(ppm) 8.02 (d, 1H, J=2.3 Hz), 7.45-7.47 (m, 1H), 3.92 (s, 3H), 2.16 (broad s, 3H).

### B) 6′-Methoxy-5′-methyl-3,4,5,6-tetrahydro-2H-[1,3′]bipyridinyl-4-carboxylic acid ethyl ester

A stirred mixture of 5-bromo-2-methoxy-3-methyl-pyridine (525 mg, 2.47 mmol) from step A, ethyl isonipecotate (466 mg, 2.96 mmol), tris(dibenzylideneacetone)palladium(0) (45 mg, 0.049 mmol), (S)-(-)-2,2-bis(diphenylphosphino)1,1-binaphtyl (62 mg, 0.099 mmol) and sodium tert-butoxide (333 mg, 3.46 mmol) was heated in anhydrous toluene (8 mL) at 70° C. under a nitrogen atmosphere for 2.5 hours. The reaction mixture was filtered through a short column of silica and the filtrate was concentrated in vacuo. The residue was purified by flash chromatography using a gradient of ethyl acetate in heptane to give the sub-title compound (235 mg, 34% yield).

¹H NMR (400 MHz; chloroform-d as solvent and internal reference) δ(ppm) 7.62 (dq, 1H, J=2.9, 0.5 Hz), 7.12 (dq, 1H, J=2.9, 0.7 Hz), 4.16 (q, 2H, J=7.1 Hz), 3.91 (s, 3H), 3.40-3.46 (m, 2H), 2.70 (ddd, 2H, J=12.1, 11.1, 2.8 Hz), 2.39 (tt, 1H, J=11.1, 8.2 Hz), 2.15-2.16 (m, 3H), 1.99-2.06 (m, 2H), 1.83-1.94 (m, 2H), 1.27 (t, 3H, J=7.1 Hz).

### C) 6′-Methoxy-5′-methyl-3,4,5,6-tetrahydro-2H-[1,3′]bipyridinyl-4-carboxylic acid hydrochloride

A solution of 6′-methoxy-5′-methyl-3,4,5,6-tetrahydro-2H- [1,3′]bipyridinyl-4-carboxylic acid ethyl ester (230 mg, 0.82 mmol) from step B and lithium hydroxide (59 mg, 2.5 mmol) in 67% aqueous tetrahydrofuran (6 mL) was stirred at room temperature for 3 hours. The reaction mixture was acidified to pH <2 by dropwise addition of 6 M hydrochloric acid. Most of the tetrahydrofuran was removed in vacuo and the remaining suspension containing the sub-title compound was freeze-dried and used without further purification.

¹H NMR (400 MHz; dimethyl sulfoxide-d₆ as solvent and internal reference) δ(ppm) 7.79 (broad s, 1H), 7.44 (broad s, 1H), 3.81 (s, 3H), 3.43-3.50 (m, 2H), 2.84-2.97 (m, 2H), 2.39-2.48 (m, 1h), 2.11 (broad s, 3H), 1.92-2.00 (m, 2H), 1.69-1.81 (m, 2H).

### D) [4-(3-Chloro-1H-indole-6-sulfonyl)-piperazin-1-yl]-(6′-methoxy-5′-methyl-3,4.5,6-tetrahydro-2H-[1,3′]bipyridinyl-4-yl)-methanone

1-Ethyl-3-(3-dimethylaminopropyl)carbodiimide hydrochloride (348 mg, 1.8 mmol) was added to a stirred solution of 6′-methoxy-5′-methyl-3,4,5,6-tetrahydro-2H-[1,3′]bipyridinyl-4-carboxylic acid hydrochloride (crude from previous step, 0.83 mmol), 3-chloro-6-(piperazine-1-sulfonyl)-1H-indole (280 mg, 0.93 mmol) and 4-dimethyl-aminopyridine (504 mg, 4.1 mmol) in N,N-dimethylformamide (8 mL) at room temperature. After stirring for 3.5 hours the reaction mixture was filtered and purified by preparative HPLC using a gradient of acetonitrile/5% acetonitrile-water phase containing 0.1 M ammonium acetate to give the sub-title compound (262 mg, 57% yield).

¹H NMR (400 MHz; dimethyl sulfoxide-d₆ as solvent and internal reference) δ(ppm) 11.85 (broad s, 1H), 7.88 (s 1H), 7.84 (dd, 1H, J=1.6, 0.6 Hz), 7.72 (dd, 1H, J=8.4, 0.6 Hz), 7.53 (dd, 1H, J=2.9, 0.6 Hz), 7.44 (dd, 1H, J=8.4, 1.6 Hz), 7.24-7.26 (m, 1H), 3.77 (s, 3H), 3.51-3.64 (m, 4H), 3.43-3.49 (m, 2H), 2.83-2.93 (m, 4H), 2.52-2.67 (m, 3H), 2.07-2.08 (m, 3H), 1.53-1.61 (m, 4H).

### E)

A mixture of [4-(3-Chloro-1H-indole-6-sulfonyl)-piperazin-1-yl]-(6′-methoxy-5′-methyl-3,4,5,6-tetrahydro-2H-[1,3′]bipyridinyl-4-yl)-methanone (144 mg, 0.27 mmol) from step D and pyridine hydrochloride (375.3 mg, 3.24 mmol) was heated at 140° C. for 9.5 minutes in a preheated oil bath and the reaction mixture was cooled to room temperature. Water and dichloromethane was added together with a small amount of acetonitrile. The layers were separated and the aqueous layer was extracted with dichloromethane. The combined organic layers were washed with water, dried, filtered and concentrated in vacuo. The crude was purified by preparative HPLC using a gradient of acetonitrile/5% acetonitrile-water phase containing 0.1 M ammonium acetate to give 101 mg (68.4% yield) of the title compound as a solid after freeze-drying.

¹H NMR (400 MHz; dimethyl sulfoxide-d₆ as solvent and internal reference) δ(ppm) 11.13 (broad s, 2H), 7.87 (s 1H), 7.83 (dd, 1H, J=1.6, 0.6 Hz), 7.72 (dd, 1H, J=8.4, 0.6 Hz), 7.43 (dd, 1H, J=8.4, 1.6 Hz), 7.26-7.28 (m, 1H), 6.51 (d, 1H, J=3.1 Hz), 3.50-3.61 (m, 4H), 3.15-3.22 (m, 2H), 2.83-2.92 (m, 4H), 2.52-2.60 (m, 1H), 2.31-2.40 (m, 2H), 1.92-1.93 (m, 3H), 1.48-1.59 (m, 4H).

HRMS (ESI+) calc. [M+H]⁺ 518.1623, found 518.1625.

### EXAMPLE 7

### 5-{4-[4-(3-Chloro-1H-indole-6-sulfonyl)-piperazine-1-carbonyl]-piperidin-1-yl}-3-methyl-1H-pyrazin-2-one

### A) 1-(5-Chloro-6-methyl-pyrazin-2-yl)-piperidine-4-carboxylic acid

2,5-Dichloro-3-methyl-pyrazine has been previously described by Sato et. al. J. Het. Chem. 1986, 871. A mixture of 2,5-dichloro-3-methyl-pyrazine (880 mg, 5.40 mmol), ethyl isonipecotate (848 mg, 5.40 mmol) and triethylamine (1.64 g, 16.2 mmol) in 25% aqueous ethanol (15 mL) was heated using single node microwave irradiation at 170° C. for 40 minutes. The solvents were concentrated in vacuo to a volume of between 3.5 mL tetrahydrofuran was added until an almost clear solution was obtained. Solid lithium hydroxide (400 mg, 16.7 mmol) was added. The resulting suspension was stirred overnight at room temperature and the solution was acidified to pH 1. The solid material was filtered off, washed with water and dried under high-vacuum to give the crude sub-title compound (734 mg, 47% yield) as a solid which was used without further purification.

¹H NMR (400 MHz; dimethyl sulfoxide-d₆ as solvent and internal reference) δ(ppm) 12.23 (broad S, 1H), 7.94 (s, 1H), 4.12-4.22 (m, 2H), 2.95-3.05 (m, 2H), 2.41-2.63 (m, 1H), 2.39 (s, 3H), 1.83-1.94 (m, 2H), 1.46-1.59 (m, 2H).

### B) 1-(6-Methyl-5-oxo-4,5-dihydro-pyrazin-2-yl)-piperidine-4-carboxylic acid

A solution of crude 1-(5-chloro-6-methyl-pyrazin-2-yl)-piperidine-4-carboxylic acid from previous step (402 mg, 1.57 mmol) and potassium acetate (3.0 g, 31 mmol) in trifluoroacetic acid (15 mL) was heated using single node microwave irradiation at 120° C. for 10 hours. After cooling the solvent was removed in vacuo. The crude was purified by preparative HPLC using first 3% acetonitrile-water phase containing 0.1 M ammonium acetate and then a gradient of acetonitrile/5% acetonitrile-water phase containing 0.1 M ammonium acetate to give the sub-title compound (88 mg, 19% yield, 80% purity) which was used without further purification.

¹H NMR (400 MHz; dimethyl sulfoxide-d₆ as solvent and internal reference) δ(ppm) 6.83 (s, 1H), 3.61-3.69 (m, 2H), 2.51-2.59 (m, 2H), 2.22 (s, 3H), 2.11-2.24 (m, 1H), 1.77-1.85 (m, 2H), 1.46-1.58 (m, 2H).

### C)

1-(6-Methyl-5-oxo-4,5-dihydro-pyrazin-2-yl)-piperidine-4-carboxylic acid from step B was treated essentially as in example 6 step D to give the title compound (30 mg, 17% yield).

¹H NMR (400 MHz; dimethyl sulfoxide-d₆ as solvent and internal reference) δ(ppm) 7.88 (s, 1H), 7.83 (d, 1H, J=1.5 Hz), 7.72 (d, J=8.4 Hz), 7.44 (dd, J=8.4, 1.5 Hz), 7.00 (broad s, 1H), 3.77-3.89 (m, 2H), 3.51-3.63 (m, 4H), 2.84-2.94 (m, 4H), 2.62-2.72 (m, 1H), 2.51-2.59 (m, 2H), 2.22 (s, 3H), 1.42-1.60 (m, 4H).

HRMS (ESI+) calc. [M+H]⁺ 519.1576, found 519.1597.

### EXAMPLE 8

### 6-{4-[4-(3-Chloro-1H-indole-6-sulfonyl)-piperazine-1-carbonyl]-piperidin-1-yl}-2-methyl-2H-pyridazin-3-one and 6-{4-[4-(1H-Indole-6-sulfonyl)-piperazine-1-carbonyl]-piperidin-1-yl}-2-methyl-2H-pyridazin-3-one (the sub-title product of step D)

### A) 4-(4-Methyl-3-nitro-benzenesulfonyl)-piperazine-1-carboxylic acid tert-butyl ester

To a solution of tert-butyl 1-piperazinecarboxylate (7.11 g, 38.2 mmol) in anhydrous dichloromethane (100 mL) was added diisopropylethylamine (9.88 g, 76.4 mmol) under nitrogen. 4-methyl-3-nitrobenzenesulfonyl chloride (9.0 g, 38 mmol) in anhydrous dichloromethane (100 mL) was added to the solution at 0° C. drop wise. The reaction mixture was stirred at room temperature over night. After addition of water, the organic phase was separated, washed with water, brine, dried and evaporated under reduced pressure to give 13.8 g (94%) of the sub-title product.

¹H NMR (500 MHz, chloroform-d as solvent and internal reference): 1.42 (s, 9H), 2.70 (s, 3H), 3.0-3.07 (m, 4H), 3.50-3.57 (m, 4H), 7.56 (d, 1H, J=8.05 Hz), 7.86 (d, 1H, J=8.05 Hz), 8.32 (s, 1H).

### B) 4-(1H-Indole-6-sulfonyl)-piperazine-1-carboxylic acid tert-butyl ester

A solution of 4-(4-methyl-3-nitro-benzenesulfonyl)-piperazine-1-carboxylic acid tert-butyl ester (6.00 g, 15.6 mmol) from step A in N,N-dimethylformamide dimethyl acetal (40 mL) containing N,N-dimethylformamide (6 mL) was heated to 100° C. for 9 hours, then evaporated to dryness. The residue was then dissolved in tetrahydrofuran (65 mL) and methanol (65 mL), and then Raney nickel (3 spoonfuls) was added. Hydrazine monohydrate (10 mL) was added dropwise, keeping the internal temperature at 45° C. The reaction mixture was stirred at 45° C. for another 2.5 hours. After addition of tetrahydrofuran and methanol, the catalyst was filtered over Celite, and the solution evaporated to dryness. The residue was suspended in ethanol, and the solids filtered to give 4.69 g (82%) of the sub-title product.

¹H NMR (500 MHz, chloroform-d as solvent and internal reference): 1.40 (s, 9H), 2.94-3.04 (m, 4H), 3.46-3.55 (m, 4H), 6.67 (s, 1H), 7.46 (s, 1H), 7.47 (d, 1H, J=8.0 Hz), 7.77 (d, 1H, J=8.0 Hz), 7.91 (s, 1H), 8.83 (s, 1H).

### C) 6-(Piperazine-1-sulfonyl)-1H-indole hydrochloride

To a mixture of 4-(1H-indole-6-sulfonyl)-piperazine-1-carboxylic acid tert-butyl ester (4.69 g, 12.8 mmol) from step B in ethanol (40 mL) was added saturated ethanol hydrogen chloride (100 mL) at 0° C. dropwise. After stirring for 105 minutes at room temperature, the solution was evaporated to dryness. The residue was suspended in ether and the solids formed were filtered to give 3.8 g (98%) of the sub-title product.

¹H NMR (300 MHz, methanol-d₄ as solvent and internal reference) δ (ppm) 3.20-3.37 (m, 8H), 6.62 (d, 1H, J=3.0 Hz), 7.43 (dd, 1H, J=1.5, 8.4 Hz), 7.56 (d, 1H, J=3.2 Hz), 7.79 (d, 1H, J=8.4 Hz), 7.92 (s, 1H).

### D) 6-{4-[4-(1H-Indole-6-sulfonyl)-piperazine-1-carbonyl]-piperidin-1-yl}-2-methyl-2H-pyridazin-3-one

To a mixture of 1-(1-methyl-6-oxo-1,6-dihydro-pyridazin-3-yl)-piperidine-4-carboxylic acid hydrochloride (80 mg, 0.29 mmol), and 6-(piperazine-1-sulfonyl)-1H-indole hydrochloride (97 mg, 0.32 mmol) from step C in anhydrous N,N-dimethylformamide (2 mL) was added diisopropylethylamine (150 mg, 1.17 mmol) and 2-(1H-benzotriazole-1-yl)-1,1,3,3-tetramethyluronium tetrafluoroborate (117 mg, 0.37 mmol) under argon. After stirring at room temperature for 30 minutes, the reaction flask was cooled to 0° C. and the reaction mixture was quenched by adding water. The solution was evaporated to dryness, and the crude product was purified by preparative HPLC using acetonitrile and ammonium acetate buffer (20:80 to 55:45) as eluent to give 140 mg (99%) of the sub-title product.

¹H NMR (500 MHz, methanol-d₄ as solvent and internal reference) δ (ppm) 1.56-1.67 (m, 4H), 2.71-2.83 (m, 3H), 2.94-3.05 (m, 4H), 3.59 (s, 3H), 3.61-3.71 (m, 4H) 2H, J=12.98 Hz), 6.59 (d, 1H, J=2.33 Hz), 6.81 (d, 1H, J=9.86 Hz), 7.36-7.43 (m, 3H), 7.52 (d, 1H, J=3.12 Hz), 7.74 (d, 1H, J=8.30 Hz), 7.87 (s, 1H).

HRMS (ESI+) calc. [M+H]⁺ 485.1965, found 485.1907.

### E) 6-{4-[4-(3-Chloro-1H-indole-6-sulfonyl)-piperazine-1-carbonyl]-piperidin-1-yl}-2-methyl-2H-pyridazin-3-one

To a solution of 6-{4-[4-(1H-indole-6-sulfonyl)-piperazine-1-carbonyl]-piperidin-1-yl}-methyl-2H-pyridazin-3-one (138 mg, 0.29 mmol) from step D in anhydrous N,N-dimethylformamide (2 mL) was added N-chlorosuccinimide (60 mg, 0.45 mmol) under argon. After stirring the reaction mixture for 4 hours and 15 minutes, the reaction flask was cooled to 0° C. and the reaction mixture was quenched by adding water. The solids formed were filtered, washed with water and purified by preparative HPLC using acetonitrile and ammonium acetate buffer (25:75 to 60:40) as eluent to give 80 mg (54%) of the title product.

¹H NMR (500 MHz, methanol-d₄ as solvent and internal reference) δ (ppm) 1.61-1.68 (m, 4H), 2.73-2.84 (m, 3H), 2.96-3.07 (m, 4H), 3.60 (s, 3H), 3.63-3.72 (m, 4H), 3.90 (d, 2H, J=13.0 Hz), 6.82 (d, 1H, J=9.9 Hz), 7.42 (d, 1H, J=10.1 Hz), 7.50 (d, 1H, J=8.6 Hz), 7.57 (s, 1H), 7.72 (d, 1H, J=8.3 Hz), 7.89 (s, 1H).

HRMS (ESI+) calc. [M+H]⁺ 519.1576, found 519.1610.

### EXAMPLE 9

### 6-{4-[4-(3-Chloro-1H-indole-6-sulfonyl)-piperazine-1-carbonyl]-piperidin-1-yl}-2H-pyridazin-3-one

### A) 3-Chloro-6-(piperazine-1-sulfonyl)-1H-indole

To a solution of 6-(piperazine-1-sulfonyl)-1H-indole hydrochloride (3.0 g, 9.94 mmol) in anhydrous N,N-dimethylformamide (9 mL) was added N-chlorosuccinimide (1.35 g, 10.1 mmol) under nitrogen. After stirring at room temperature for 40 minutes, the reaction flask was cooled to 0° C. and the reaction mixture was quenched by adding water. The solution was made alkaline by adding sodium bicarbonate, and solid material precipitated. The solids was filtered, washed with water, ethanol, ether and dried in vacuo to give 2.5 g (84%) of 3-chloro-6-(piperazine-1-sulfonyl)-1H-indole.

¹H NMR (300 MHz, methanol-d₄ as solvent and internal reference) δ (ppm) 3.20-3.30 (m, 8H), 7.54 (dd, 1H, J=1.7, 8.6 Hz), 7.60 (s, 1H), 7.77 (dd, 1H, J=0.5, 8.4 Hz), 7.94 (d, 1H, J=0.5 Hz).

The hydrochloride salt was optionally prepared by adding 1 M hydrochloric acid to the neutral form dissolved in methanol followed by removal of solvents in vacuo.

### B)

To a mixture of 1-(6-oxo-1,6-dihydro-pyridazin-3-yl)-piperidine-4-carboxylic acid (60 mg, 0.27 mmol), and 3-chloro-6-(piperazine-1-sulfonyl)-1H-indole hydrochloride (90 mg, 0.27 mmol) from step A in anhydrous N,N-dimethylformamide (2 mL) was added diisopropyl-ethylamine (87 mg, 0.67 mmol) and 2-(1H-benzotriazole-1-yl)-1,1,3,3-tetramethyluronium tetrafluoroborate (117 mg, 0.37 mmol). After stirring at room temperature for 50 minutes, the reaction flask was cooled to 0° C. and the reaction mixture was quenched by adding water. The solids formed were filtered, washed with water and purified by column chromatography on silica gel using dichloromethane/methanol (100:4 and 100:7) as eluent to give 70 mg (52%) of the title product.

¹H NMR (500 MHz, methanol-d₄ as solvent and internal reference) δ (ppm) 1.59-1.71 (m, 4H), 2.72-2.84 (m,3H), 2.96-3.08 (m, 4H), 3.61-3.73 (m, 4H), 3.82-3.93 (m, 2H), 6.80-6.87 (m, 1H), 7.44-7.54 (m, 2H), 7.57 (d, 1H, J=7.0 Hz), 7.70-7.76 (m, 1H), 7.89 (d, 1H, J=5.4 Hz).

HRMS (ESI+) calc. [M+H]⁺ 505.1419, found 505.1440.

### EXAMPLE 10

### 6-{4-[4-(3-Chloro-1H-indole-6-sulfonyl)-benzoyl]-piperazin-1-yl}-2-methyl-2H-pyridazin-3-one

### A) 4-(1H-Indol-6-ylsulfanyl)-benzoic acid

A 20 mL microwave-vial was charged with 1.1 g of 6-bromo-1H-indole (5.6 mmol), 1.87 g of sodium iodide (12.5 mmol), 0.12 g of copper (I) iodide (0.62 mmol), a stirring bar and butyl-rubber septum. The vial was evacuated and backfilled with argon. This was repeated twice. A solution of 133 μL N,N′-dimethylethylenediamine (0.11 g, 1.2 mmol) in 5 mL of dioxane was injected into the vial. The vial was then capped and heated in a microwave oven at 130° C. for 1.5 hours. After cooling to room temperature, 123 mg of copper(I)iodide (0.65 mmol), 0.622 g of sodium iodide (4.15 mmol), 0.117 g of neocuproine (0.56 mmol), 0.944 g of 4-mercapto-benzoic acid methyl ester (5.61 mmol) and 0.809 g of sodium tert-butoxide (8.41 mmol) were added to the above resulting mixture. The mixture was heated again using microwaves at 130° C. for 4 hours. After cooling to room temperature, the reaction mixture was diluted with lithium hydroxide aqueous solution, prepared freshly from 0.62 g of lithium hydroxide (26 mmol) and 40 mL of water. The resulting mixture was stirred overnight at room temperature, extracted with 3×50 mL of dichloromethane. The aqueous layers were combined, and acidified with 4 M aqueous hydrochloride. The precipitated solid was collected and dried in vacuo. The dried crude product was treated with 100 mL of methanol to remove insoluble residues. After filtration, the filtrate was concentrated and dried in vacuo. The residue was subjected to reversed phase preparative HPLC for purification After freeze drying, 0.20 g (13%) of the sub-title product was obtained as a white powder.

¹H NMR (400 MHz; methanol-d₄ as solvent and internal reference) δ(ppm) 6.50 (d, 1H, J=3.2 Hz), 7.06 (d, 1H, J=8.5 Hz), 7.14 (dd, 1H, J=8.3, 1.4 Hz,), 7.32 (d, 1H, J=3.2 Hz), 7.59-7.63 (m, 2H), 7.80 (d, 1H, J=8.5 Hz).

### B) 4-(1H-Indole-6-sulfonyl)-benzoic acid

4-(1H-Indol-6-ylsulfanyl)-benzoic acid (0.45 g, 1.7 mmol) from step A was mixed in 20 mL of dry methanol. The mixture was cooled with ice-water bath. Caro's acid was added potion wise to the mixture. The mixture was stirred continuously with the ice-water bath for 1.5 hour, and then warmed to room temperature. The mixture was stirred for 2 days at room temperature until all sulfoxide converted into sulfonyl according to LCMS. After evaporation, the residue was mixed with 5 mL of N,N-dimethylformamide. The mixture was filtered, and the N,N-dimethylformamide solution was loaded onto reversed phase preparative HPLC column for purification. After freeze drying, 0.44 g (87%) of the sub-title product was obtained as a white powder.

¹H NMR (400 MHz; methanol-d₄ as solvent and internal reference) δ(ppm) 6.57 (d, 1H, J=3.1 Hz), 7.53 (d, 1H, J=2.8 Hz), 7.55 (dd, 1H, J=8.4, 1.6 Hz,), 7.72 (d, 1H, J=8.4 Hz), 7.95 (d, 2H, J=8.4 Hz), 8.05 (d, 2H, J=8.4 Hz), 8.09 (s, 1H).

### C) 4-(3-Chloro-3a,7a-dihydro-1H-indole-6-sulfonyl)-benzoic acid

4-(1H-Indole-6-sulfonyl)-benzoic acid (0.381 g, 1.26 mmol) from step B and 0.186 g of N-chlorosuccinimide (1.39 mmol) were mixed in 4 mL of dry N,N-dimethylformamide at 0° C. The solution was stirred at 0° C. for 30 minutes, warmed to room temperature and stirred for 1 hour. Then the mixture was heated at 80° C. for 3 hours. The mixture was loaded directly onto a reversed phase preparative HPLC for purification. After freeze drying, 0.28 g (66%) of the sub-title product was obtained as a white powder.

¹H NMR (400 MHz; methanol-d₄ as solvent and internal reference) δ(ppm) 7.55 (s, 1H), 7.63 (dd, 1H, J=8.4 Hz, J=1.3 Hz,), 7.67 (d, 1H, J=8.4 Hz), 8.00 (d, 2H, J=8.3 Hz), 8.10 (s, 1H), 8.12 (d, 2H, J=8.3 Hz).

### D)

2-Methyl-6-piperazin-1-yl-2H-pyridazin-3-one trifluoroacetate (50 mg, 0.12 mmol) was mixed with 0.10 mL of N,N-diisopropylethylamine (0.57 mmol) in 3 mL of dry N,N-dimethylformamide. 55 mg of 4-(3-chloro-3a,7a-dihydro-1H-indole-6-sulfonyl)-benzoic acid (0.16 mmol), from Example 1 step C, was mixed with 231 mg of O-(benzotriazol-1-yl)-N,N,N′,N′-tetramethyluronium tetrafluoroborate (0.72 mmol) in dry N,N-dimethylformamide at room temperature, and the mixture was stirred for 20 minutes. The basic 2-methyl-6-piperazin-1-yl-2H-pyridazin-3-one solution was then added to the activated carboxylic acid solution at room temperature. The mixture was continuously stirred overnight. The reaction mixture was loaded onto reversed phase preparative HPLC for purification. After freeze drying, 10 mg (17%) of the product was obtained as a white powder.

¹H NMR (400 MHz; N,N-dimethylformamide-d₇ as solvent and internal reference) δ(ppm) 3.18-3.99 (m, 8H), 3.51 (s, 3H), 6.84 (d, 1H, J=10.3 Hz), 7.53 (d, 1H, J=10.3 Hz), 7.66-7.77 (m, 4H), 7.92 (s, 1H), 8.10 (d, 2H, J=8.4 Hz), 8.26 (s, 1H).

HRMS (ESI+) calc. [M+H]⁺ 512.1154, found 512.1150.

### EXAMPLE 11

### 6-{4-[4-(6-Bromo-naphthalene-2-sulfonyl)-benzoyl]-piperidin-1-yl}-2-methyl-2H-pyridazin-3-one

### A) [4-(6-Bromo-naphthalene-2-sulfonyl)-phenyl]-piperidin-4-yl-methanone hydrochloride

A mixture of HCl-saturated methanol and 4-[4-(6-bromo-naphthalene-2-sulfonyl)-benzoyl]-piperidine-1-carboxylic acid tert-butyl ester (190 mg, 0.34 mmol) was stirred at 60° C. for 20 minutes. Solvent was removed with evaporator in vacuo to give 155 mg (92%) of [4-(6-bromo-naphthalene-2-sulfonyl)-phenyl]-piperidin-4-yl-methanone hydrochloride.

¹H NMR (500 MHz, dimethyl sulfoxide-d₆ as solvent and internal reference) δ(ppm) 1.63-1.74 (m, 2H), 1.87-1.94 (m, 2H), 2.93-3.02 (m, 2H), 3.32-3.30 (m, 2H), 3.74 (tt, 1H. J=3.4, 11.1 Hz), 7.84 (dd, 1H, J=2.0, 8.8 Hz), 8.00 (dd, 1H, J=1.9, 8.8 Hz), 8.13 (d, 1H, J=8.8 Hz), 8.17 (s, 4H), 8.19 (d, 1H, J=8.8 Hz), 3.37 (d, 1H, J=1.7 Hz), 8.53-8.56 (m, 1NH), 8.80 (d, 1H, J=1.5 Hz), 8.83-8.96 (m, 1NH).

### B)

[4-(6-Bromo-naphthalene-2-sulfonyl)-phenyl]-piperidin-4-yl-methanone hydrochloride (130 mg, 0.26 mmol) from step A, 6-chloro-2-methyl-2H-pyridazin-3-one (57 mg, 0.39 mmol) and triethylamine (0.22 mL, 1.6 mmol) were mixed together with a magnetic stirrer bar and the solvent (ethanol/water 3:1, 4 mL) in a microwave vial. The reaction mixture was heated in a microwave oven to 180° C. for 35 hours. Solvent was removed with evaporator in vacuo. The residue was dissolved in dimethyl sulfoxide (3 mL) and purified by preparative HPLC using a gradient of acetonitrile/5% acetonitrile-water phase containing 0.1 M ammonium acetate. Evaporation and freeze drying gave 40 mg (27%) of the title compound.

¹H NMR (500 MHz, chloroform-d as solvent and internal reference) δ(ppm) 1.78-1.88 (m, 2H), 1.90-1.97 (m, 2H), 2.90-2.97 (m, 2H), 3.40 (tt, 1H, J=3.8, 10.9 Hz), 3.66 (s, 3H), 3.85 (dt, 2H, J=13.3, 3.4 Hz), 6.98 (d, 1H, J=9.9 Hz), 7.16 (d, 1H, J=9.9 Hz), 7.71 (dd, 1H, J=1.9, 8.7 Hz), 7.86 (d, 2H, J=8.0 Hz), 7.88 (dd, 1H, J=1.7, 8.8 Hz), 8.04 (d, 2H, J=8.6 Hz), 8.07 (d, 1H, J=1.5 Hz), 8.10 (d, 1H, J=8.6 Hz), 8.56 (s, 1H).

HRMS (ESI+) calc. [M+H]⁺ 566.0743, found 566.0690.

### EXAMPLE 12

### 6-(4-{4-[(E)-2-(5-bromo-thiophen-2-yl)-ethenesulfonyl]-piperazine-1-carbonyl}-piperidin-1-yl)-2H-pyridazin-3-one

### A) Methanesulfonic acid butyl ester

n-Butanol (6.7 g, 8.2 mL, 0.090 mol) was dissolved in 30 mL anhydrous methylene chloride and diisopropylethylamine (19.4 g, 26.1 mL, 0.150 mol) was added. The solution was cooled to −5° C. and methanesulfonyl chloride (11.4 g, 7.74 mL, 0.100 mol) was added dropwise followed by stirring for 0.5 hours at −5 to 0° C. After 0.5 hours, the reaction was quenched with ice cold water. The crude mixture was washed with ice cold water, cold 10% hydrochloric acid, followed by cold water and then cold sodium bicarbonate solution and finally with cold brine. The organic phase was dried over anhydrous magnesium sulfate and the solvent was removed by evaporation. The sub-title product (13.8 g, 91%) was extracted as slightly brownish liquid after removal of solvents in vacuo. The sub-title product was used in the next step without further purification.

¹H NMR (400 MHz, chloroform-d as solvent and internal reference) δ (ppm) 4.19 (t, 2H, J=6.5 Hz), 2.96 (s, 3H), 1.69 (m, 2H), 1.40 (m, 2H), 0.91 (t, 2H, J=7.3 Hz).

### B) (E)-2-(5-Bromo-thiophen-2-yl)-ethenesulfonic acid butyl ester

n-Butyl methane sulfonate (3.045 g, 20 mmol) from step A was dissolved in 80 mL tetrahydrofuran and cooled to −78° C. n-Butyl lithium (2.5 M in hexane, 8.8 mL, 22 mmol) was added dropwise. The mixture was stirred for 30 minutes. Diethylchlorophosphate (3.45 g, 2.9 mL, 20 mmol) was added dropwise. The mixture was stirred for 0.5 hours at −78° C. and then heated to 50° C. for 1h. The light orange colour solution was cooled back to −78° C. and n-butyllithium (2.5 M in hexane, 22 mmol, 8.8 mL) was added dropwise. The colour of the reaction became dark orange. After stirring at −78° C. for 0.5 h, a solution of 5-bromothiophene-2-carboxaldehyde (3.821 g in 5 mL anhydrous tetrahydrofuran) was added to the reaction mixture. Water was added with a syringe to quench the reaction. It was stirred for 10 minutes at 0° C. Tetrahydrofuran was evaporated and the mixture was diluted with 200 mL dichloromethane. It was worked up using dichloromethane, water and brine, dried over anhydrous magnesium sulfate and evaporated. The sub-title product was purified using a flash chromatography on silica gel using hexane, 5% and 10% ethyl acetate in hexane.

¹H NMR (400 MHz, chloroform-d as solvent and internal reference) δ (ppm) 7.54 (d, 1H, J=15.2), 7.04 (dd, 2H, J=7.3 Hz), 6.40 (d, 1H, J=15.2), 4.12 (t, 2H, J=6.51), 1.68 (m, 2H), 1.39 (m, 2H), 0.91 (t, 3H, J=7.5)?

### C) Tetrabutylammonium (E)-2-(5-bromo-thiophen-2-yl)-ethenesulfonate

(E)-2-(5-Bromo-thiophen-2-yl)-ethenesulfonic acid butyl ester (1.5 g, 4.6 mmol) from step B and tetrabutylammonium iodide (1.88 g, 5.07 mmol) were dissolved in 50 mL acetone, and refluxed overnight. The mixture was cooled to room temperature and concentrated under vacuum. The residue was dissolved in dichloromethane and washed with water and brine. The organic layer was dried over anhydrous magnesium sulfate and evaporated to yield 2.3 g (98%) of the sub-title product.

¹H NMR (400 MHz, chloroform-d as solvent and internal reference) δ (ppm) 7.22 (d, 1H, J=15.4), 6.86 (d, 1H, 3.8), 6.72 (d, 1H, J=3.8), 6.67 (d, 1H, J=15.4), 3.25 (t, 8H, J=8.3), 1.60 (m, 8H), 1.39 (m, 8H), 0.94 (t, 12H, J=7.4)?

### D) (E)-2-(5-Bromo-thiophen-2-yl)-ethenesulfonyl chloride

Triphenylphosphine (2.48 g, 9.45 mmol) was dissolved in 30 mL dichloromethane and cooled to 0° C. Sulfuryl chloride (1.34 g; 0.80 mL, 9.90 mmol) was added. After stirring for 5 minutes at that temperature, tetrabutylammonium (E)-2-(5-bromo-thiophen-2-yl)-ethenesulfonate (2.3 g, 4.5 mmol) from step C solution in 20 mL dichloromethane was added dropwise at 0° C. The cooling bath was removed and the mixture was stirred at room temperature for 2 hours. The solvent was removed by evaporation and the crude material sub-title product was purified by flash chromatography on silica gel using hexane and then ethyl acetate/hexane to give 0.54 g (42%) of the the sub-title compound.

¹H NMR (400 MHz, chloroform-d as solvent and internal reference) δ (ppm) 7.69 (d, 1H, J=15.0 Hz), 7.18 (d, 1H, J=3.9 Hz), 7.11 (d, 1H, J=4.0 Hz), 6.93 (d, 1H, J=14.8 Hz).

### E)

The title product was synthesized and purified essentially as described in step G of Example 13, but with a reaction time of 20 minutes using 6-[4-piperazine-1-carbonyl)-piperidin-1-yl]-2H-pyridazin-3-one hydrochloride (76 mg, 0.23 mmol) and (E)-2-(5-bromo-thiophen-2-yl)-ethenesulfonyl chloride (67 mg, 0.23 mmol) as starting material to give 47 mg (37%).

¹H NMR (500 MHz, dimethyl sulfoxide-d₆ as solvent and internal reference) δ(ppm) 1.50-1.66 (m, 4H), 2.71 (dt, 2H, J=2, 12 Hz), 2.76-2.83 (m, 1H), 3.01-3.10 (m, 4H), 3.52-3.66 (m, 4H), 3.77 (broad d, 1H, J=13 Hz), 6.73 (d, 1H, J=10 Hz), 6.98 (d, 1H, J=15 Hz), 7.30 (d, 1H, J=4 Hz), 7.45 (d, 1H, J=4 Hz), 7.47 (d, 1H, J=10 Hz), 7.53 (d, 1H, J=15 Hz), 12.03 (broad s, 1NH).

HRMS (ESI+) calc. [M+H]⁺ 542.0526, found 542.0509.

### EXAMPLE 13

### 6-(4-{4-[(E)-1-(5-chloro-thiophen-2-yl)-prop-1-ene-2-sulfonyl]-piperazine-1-carbonyl}-piperidin-1-yl)-2H-pyridazin-3-one

### A) Ethanesulfonic acid butyl ester

n-Butanol (1.82 g, 2.24 mL, 24.5 mmol) was dissolved in 30 mL anhydrous methylene chloride and diisopropylethylamine (5.28 g, 7.11 mL, 40.8 mmol) was added. The solution was cooled to −5° C. Ethenesulfonyl chloride was added dropwise and the reaction was stirred for 0.5 hours. The mixture was washed with ice-cold water, cold 10% hydrochloric acid, followed by cold water and then cold sodium bicarbonate solution and finally with cold brine. The organic phase was dried over anhydrous magnesium sulfate and the solvent was removed by evaporation. 4.05 g (99%) slightly brownish liquid was extracted. The sub-title product was used for the next step without further purification.

¹H NMR (400 MHz, chloroform-d as solvent and internal reference) δ (ppm) 4.20 (t, 2H, J=6.6), 3.09 (q, 2H, J=7.5), 1.70 (m, 2H), 1.41 (m, 2H), 1.39 (t, 3H, J=7.4), 0.95 (t, 3H, J=7.4).

### B) (E)-1-(5-Chloro-thiophen-2-yl)propene-2-sulfonic acid butyl ester

Ethanesulfonic acid butyl ester was used directly from previous reaction, i.e. from step A, without any extraction or purification. The light orange colour solution was cooled to −78° C. and then n-butyllithium (2.5 M in hexane, 22 mmol, 8.8 mL) was added dropwise. The clear orange colour of the reaction became darker cloudy orange. After stirring at −78° C. for 0.5 hours, a solution of 5-chlorothiophene-2-carboxaldehyde (2.93 g in 5 mL anhydrous tetrahydrofuran) was added to the reaction mixture. The mixture was left stirring overnight and the temperature was slowly brought up to above 0° C. Water (30 mL) was added with a syringe and the mixture was stirred for 10 minutes at 0° C. The tetrahydrofuran was evaporated in vacuo and the mixture was diluted with dichloromethane (200 mL). Extractive work up using dichloromethane, water and brine, dried over anhydrous magnesium sulfate and evaporated. Flash chromatography on silica gel (gradient) using pure hexane to ethyl acetate/hexane 1:1 was used to get the sub-title product together with the minor Z-isomer. The mixture was used directly in the next step.

¹H NMR (400 MHz, chloroform-d as solvent and internal reference) δ (ppm) 7.57 (s, 1H), 7.09 (d, 1H, J=4.0 Hz), 6.96 (d, 1H, J=3.9 Hz), 4.07 (t, 2H, J=6.5 Hz), 2.25 (s, 3H), 1.68 (m, 2H), 1.40 (m, 2H), 0.91 (t, 3H, J=7.4 Hz).

### C) (E)-1-(5-Chloro-thiophen-2-yl)propene-2-sulfonatetetra-butyl-ammonium

(E)-1-(5-Chloro-thiophen-2-yl)propene-2-sulfonic acid butyl ester (1.50 g, 5.09 mmol) from step B and tetrabutylammonium iodide (2.07 g, 5.60 mmol) were dissolved in 50 mL acetone, and refluxed overnight. The mixture was cooled to room temperature and concentrated under vacuum. It was then dissolved in dichloromethane and washed with water and brine. The organic layer was dried over anhydrous magnesium sulfate, filtered and evaporated. The resulting the sub-title compound was used further in step D).

¹H NMR (400 MHz, chloroform-d as solvent and internal reference) δ (ppm) 7.34 (s, 1H), 6.77 (s, 2H), 3.25 (t, 8H, J=8.4 Hz), 2.24 (s, 3H), 1.60 (m, 8H), 1.39 (m, 8H), 0.96 (t, 12H, J=7.3 Hz).

### D) (E)-1-(5-Chloro-thiophen-2-yl)propene-2-sulfonyl chloride

Triphenylphosphine (2.754 g, 10.5 mmol) was dissolved in 30 mL dichloromethane and cooled to 0° C. Sulfuryl chloride (1.53 g, 0.92 mL, 11.0 mmol) was added. Tetrabutylammonium salt of chlorothiophene vinyl sulfonate (2.4 g, 5.0 mmol) from step C was dissolved in 20 mL dichloromethane and added to the above mixture at 0° C. The cooling bath was removed and the mixture was stirred at room temperature for 2 hours. Progress of the reaction was checked by using LC-MS Making amide of the sulfonyl chloride. The solvent was removed and the crude was purified by flash column chromatography (70 g Isolute SPE column) eluting with ethyl acetate/hexane 1:20. The sub-title product (0.345 g, 27%) was isolated after removal of solvents in vacuo.

¹H NMR (400 MHz, chloroform-d as solvent and internal reference) δ (ppm) 7.72 (s, 1H), 7.21 (d, 1H, J=4.0 Hz), 7.02 (d, 1H, J=4.0 Hz), 2.44 (s, 3H).

### E) 4-[1-(6-Oxo-1,6-dihydro-pyridazin-3-yl)-piperidine-4-carbonyl]-piperazine-1-carboxylic acid tert-butyl ester

To a suspension of 1-(6-oxo-1,6-dihydro-pyridazin-3-yl)-piperidine-4-carboxylic acid (500 mg, 2.24 mmol) and piperazine-1-carboxylic acid tert-butyl ester (626 mg, 3.36 mmol) in dry N,N-dimethylformamide (30 mL,) at room temperature was added 4-dimethyl aminopyridine (684 mg, 5.60 mmol) and O-(benzotriazol-1-yl)-N,N,N′N′-tetramethyluronium tetrafluoroborate (1.44 g, 4.48 mmol). The reaction mixture was stirred at room temperature for 30 minutes and then concentrated in vacuo. The residue was purified with HPLC using a gradient of acetonitrile/5% acetonitrile-water phase containing 0.1 M ammonium acetate, to give 510 mg (58%) of the sub-title compound after evaporation and freeze drying over night.

¹H NMR (400 MHz, dimethyl sulfoxide-d₆ as solvent and internal reference) δ(ppm) 1.40 (s, 9H), 1.51-1.68 (m, 4H), 2.65-2.87 (m, 3H), 3.23-3.53 (m, 8H), 3.79 (broad d, 2H, J=13 Hz), 6.74 (d, 1H, J=10 Hz), 7.48 (d, 1H, J=10 Hz), 12.04 (s, 1H).

### F) 6-[4-(Piperazine-1-carbonyl)-piperidin-1-yl]-2H-pyridazin-3-one hydrochloride

To a solution of 4-[1-(6-oxo-1,6-dihydro-pyridazin-3-yl)-piperidine-4-carbonyl]-piperazine-1-carboxylic acid tert-butyl ester (0.47 g, 1.20 mmol) from step E in dry methanol (10 mL) was added hydrochloric acid-saturated methanol (10 mL). After 30 minutes the solvent was removed with evaporator in vacuo to give 388 mg (99%) of the sub-title product.

¹H NMR (400 MHz, dimethyl sulfoxide-d₆ as solvent and internal reference) δ(ppm) 1.57 (dq, 2H, f=3, 12 Hz), 1.61-1.70 (m, 2H), 2.74 (broad t, 2H, J=12 Hz), 2.78-2.87 (m, 1H), 2.98-3.12 (m, 4H), 3.61-3.83 (m, 6H), 6.76 (d, 1H, J=10 Hz), 7.50 (d, 1H, J=10 Hz), 9.36 (broad s, 2NH), 12.09 (broad s, 1NH).

### G)

To a mixture of 6-[4-(piperazine-1-carbonyl)-piperidin-1-yl]-2H-pyridazin-3-one hydrochloride (0.10 g, 0.31 mmol) from step F in anhydrous N,N-dimethylformamide (2 mL) was added 4-dimethyl aminopyridine (149 mg, 1.22 mmol) at 0° C. under nitrogen. To the mixture, a solution of (E)-1-(5-chloro-thiophen-2-yl)-propene-2-sulfonyl chloride (366 mg, 0.94 mmol) from step D in anhydrous N,N-dimethylformamide (1 mL) was added at 0° C., and the reaction mixture was stirred at room temperature for 40 minutes. The solvent was removed in vacuo before purification with HPLC using a gradient of acetonitrile/5% acetonitrile-water phase containing 0.1 M ammonium acetate, to give 112 mg (72%) of the title compound, after evaporation and freeze drying over night.

¹H NMR (400 MHz, dimethyl sulfoxide-d₆ as solvent and internal reference) δ(ppm) 1.49-1.67 (m, 3H), 2.17 (s, 3H), 2.64-2.83 (m, 3H), 3.04-3.16 (m, 4H), 3.48-3.64 (m, 4H), 3.77 (broad d, 2H, J=13 Hz), 6.73 (d, 1H, J=10 Hz), 7.28 (d, 1H, J=4 Hz), 7.47 (d, 1H, J=10 Hz), 7.48 (d, 1H, J=4 Hz), 7.51 (s, 1H), 12.03 (broad s, 1NH).

HRMS (ESI+) calc. [M+H]⁺ 512.1188, found 512.1188.

### EXAMPLE 14

### 6-{1-[1-(5-chloro-1H-indole-2-sulfonyl)-piperidine-4-carbonyl]-piperazin-4-yl}-2-methyl-2H-pyridazin-3-one

### A) Piperazin-4-yl-2-methyl-2H-pyridazin-3-one hydrochloride

6-Chloro-2-methyl-2H-pyridazin-3-one (290 mg, 2.00 mmol), piperazine (175 mg, 2.03 mmol) and pyridine (480 mg, 6.07 mmol) were dissolved in ethanol/water (3:1, 4 mL) and put into a microwave vial together with a magnetic stirrer bar. The reaction mixture was heated in a microwave oven to 180° C. for 15 hours. The solvent was removed in vacuo and the residue purified on silica gel (60 mesh, immobilised with methanol and dried) using dichloromethane/ammonia saturated methanol (0-30%) as eluent. After pooling of selected fractions and evaporation solvents in vacuo, the compound was converted to the hydrochloride by dissolution in methanol (5 mL) and adding 1 eq. of 1 M hydrochloric acid. Evaporation of solvents in vacuo gave 232 mg (50%) of the sub-title product.

¹H NMR (300 MHz, chloroform-d as solvent and internal reference) d(ppm): 3.35-3.42 (m, 4H), 3.61-3.66 (m, 4H), 3.68 (s, 3H), 7.01 (d, 2H, J=10 Hz), 7.52 (d, 2H, J=10 Hz).

### B)

Piperazin-4-yl-2-methyl-2H-pyridazin-3-one hydrochloride (30 mg, 0.13 mmol) from step A, 1-(5-chloro 1H-indole-2-sulfonyl)piperidine-4-carboxylic acid (40 mg, 0.12 mmol), O-(benzotriazol-1-yl)-N,N,N′,N′-tetramethyluronium tetrafluoroborate (42 mg, 0.13 mmol) and dimethylaminopyridine (49 mg, 0.39 mmol) were dissolved in dry N,N-dimethyl-formamide. The reaction mixture was stirred at ambient temperature for 16 hours. The solvent was removed in vacuo. The residue was dissolved in dichloromethane (100 mL, washed twice with 0.3 M potassium hydrogensulfate (aq, 50 mL,), water (50 mL), 1 M sodium bicarbonate (aq, 50 mL), water (50 mL) and finally with brine (50 mL). The organic layer was dried over sodium sulfate and after filtration the solvent was removed in vacuo. The residue was purified by HPLC (kromasil C-18) using a gradient of acetonitrile/5% acetonitrile water phase containing 0.1 M ammoinium acetate, to give 43 mg (64%) of the title product after evaporation and freeze drying over night.

¹H NMR (300 MHz, acetonitrile-d₃ as solvent and internal reference) d(ppm): 1.75-1.90 (m, 4H), 2.45-2.59 (m, 2H), 2.59-2.70 (m, 1H), 3.25-3.37 (m, 4H), 3.55-3.77 (m, 4H), 3.66 (s, 3H), 3.79-3.89 (m, 2H), 6.99 (s, 1H), 7.18 (d, 1H, J=10.5 Hz), 7.30 (d, 1H, J=9.2 Hz), 7.40 (d, 1H, J=10.5 Hz), 7.47 (d, 1H, J=9.2 Hz), 7.69 (s, 1H).

### EXAMPLE 15

### 6-{1-[1-(5-Chloro-1H-indole-2-sulfonyl)-piperidine-4-carbonyl]-piperidin-4-yl}-2-methyl-2H-pyridazin-3-one

### A) 4-(1-Methyl-6-oxo-1,6-dihydro-pyridazin-3-yl)-3,6-dihydro-2H-pyridine-1-carboxylic acid tert-butyl ester

A mixture of 4-(4,4,5,5-tetramethyl-[1,3,2]dioxaborolan-2-yl)-3,6-dihydro-2H-pyridine-1-carboxylic acid tert-butyl ester (Eastwood, P. R., Tetrahedron Lett. 2000, 41, 3705.) (270 mg, 0.873 mmol), 6-chloro-2-methyl-2H-pyridazin-3-one (133 mg, 0.917 mmol), potassium carbonate (362 mg, 2.62 mmol), and [1,1′-bis(diphenylphosphino)ferrocene]-dichloropalladium(II), complex with dichloromethane (1:1) (43 mg, 52 μmol) in dry and degassed N,N-dimethylformamide (6 mL) was stirred under argon at 80° C. After 15 hours the reaction mixture was diluted with ethyl acetate and filtered (Celite). The filtrate was washed twice with water, and saturated aqueous NaCl, dried over sodium sulfate and concentrated in vacuo. Flash column chromatography (heptane/ethyl acetate 1:39→0:1) of the residue gave 162 mg (64%) of the sub-title product.

¹H NMR (500 MHz, chloroform-d as solvent and internal reference) δ (ppm) 1.50 ( s, 9H), 2.58 (broad s, 2H), 3.59-3.62 (m, 2H), 3.79 (s, 3H), 4.13 (broad s, 2H), 6.24 (broad s, 1H), 6.91 (d, 1H, J=9.6 Hz), 7.49 (d, 1H, J=9.6 Hz).

### B) 4-(1-Methyl-6-oxo-1,6-dihydro-pyridazin-3-yl)-piperidine-1-carboxylic acid tert-butyl ester

A solution of 4-(1-methyl-6-oxo-1,6-dihydro-pyridazin-3-yl)-3,6-dihydro-2H-pyridine-1-carboxylic acid tert-butyl ester (154 mg, 0.529 mmol) from step A in ethanol (5 mL) was subjected to hydrogenolysis (1 bar) over 10% Pd(C) (51 mg) at room temperature for 18 hours. The catalyst was then filtered off (Celite) and washed with ethanol. The combined filtrates were concentrated in vacuo and flash column chromatography on silica gel (ethyl acetate) of the residue gave 78 mg (50%) of the sub-title product.

¹H NMR (400 MHz, chloroform-d as solvent and internal reference) δ (ppm) 1.45 (s, 9H), 1.52-1.63 (m, 2H), 1.78-1.84 (m, 2H), 2.60-2.68 (m, 1H), 2.74-2.83 (m, 2H), 3.72 (s, 3H), 4.15-4.24 (m, 2H), 6.87 (d, 1 H, J=9.5 Hz), 7.10 (d, 1 H, J=9.5 Hz).

### C) 1-(1-Benzenesulfonyl-5-chloro-1H-indole-2-sulfonyl)-piperidine-4-carboxylic acid ethyl ester

A solution of 1-benzenesulfonyl-5-chloro-1H-indole-2-sulfonyl chloride (1.117 g, 3.00 mmol) in dichloromethane (10 mL) was added to a solution of ethyl isonipecotate (462 μL, 3.00 mmol), and N,N-diisopropylethylamine (1.05 mL, 6.00 mmol) in dichloromethane (10 mL) at 0° C. The reaction mixture was stirred at 0° C. for 10 minutes and at room temperature for 18 hours. The mixture was then diluted with dichloromethane, washed twice with 1 M aqueous hydrochloric acid, and water, dried over sodium sulfate and concentrated in vacuo. Flash column chromatography heptane/ethyl acetate 3:2) of the residue gave 840 mg (55%) of the sub-title product after removal of solvents in vacuo.

¹H NMR (400 MHz, chloroform-d as solvent and internal reference) δ (ppm) 1.23 (t, 3H, J=7.1 Hz), 1.77-1.87 (m, 2H), 1.95-2.01 (m, 2H), 2.40-2.47 (m, 1H), 2.99-3.07 (m, 2H), 3.80-3.86 (m, 2H), 4.13 (q, J=7.1 Hz, 2H), 7.37 (m, 4H), 7.51-7.55 (m, 2H), 7.98-8.01 (m, 2H), 8.20 (d, 1H, J=9.1 Hz).

### D) 1-(5-Chloro-1H-indole-2-sulfonyl)-piperidine-4-carboxylic acid

A solution of sodium hydroxide (125 mg, 3.13 mmol) in water (3 mL) was added to a solution of 1-(1-benzenesulfonyl-5-chloro-1H-indole-2-sulfonyl)-piperidine-4-carboxylic acid ethyl ester (400 mg, 0.783 mmol) from step C in 1-methyl-2-pyrrolidinone (9 mL). The reaction mixture was heated with microwaves in a single-mode instrument at 130° C. during 10 minutes. The mixture was then subjected to reversed phase preparative HPLC (10→100% acetonitrile in 0.1 M aqueous anunonium acetate) to give 214 mg (80%) of the sub-title product.

¹H NMR (400 MHz, dimethyl sulfoxide-d₆ as solvent and internal reference) 1.45-1.56 (m, 2H), 1.81-1.87 (m, 2H), 2.21-2.29 (m, 1H), 2.49-2.56 (m, 2H), 3.47-3.52 (m, 2H), 6.94 (s, 1H), 7.27 (dd, 1H, J=2.0, 8.9 Hz), 7.44 (d, 1H, J=8.9 Hz), 7.73 (d, 1H, J=2.0 Hz), 12.25 (broad s, 2H).

### E)

Trifluoroacetic acid (2 mL) was added to a solution of 4-(1-methyl-6-oxo-1,6-dihydro-pyridazin-3-yl)-piperidine-1-carboxylic acid tert-butyl ester (77 mg, 0.262 mmol) from step B in dichloromethane (2 mL) and the reaction mixture was stirred at room temperature for 90 minutes. Toluene was then added and the solvents were then evaporated to give the crude trifluoroacetate salt of 2-methyl-6-piperidin-4-yl-2H-pyridazin-3-one. The crude trifluoroacetate salt was dissolved in N,N-dimethylformamide (4 mL) and treated with N,N-diisopropylethylamine (91 μL, 0.52 mmol). To a solution of 1-(5-chloro-1H-indole-2-sulfonyl)-piperidine-4-carboxylic acid (108 mg, 0.315 mmol) in N,N-dimethylformamide (6 mL) was added N,N-diisopropylethylamine (91 μL, 0.52 mmol) and O-(benzotriazol-1-yl)-N,N,N′,N′-tetramethyluronium tetrafluoroborate (101 mg, 0.321 mmol) and the reaction mixture was stirred at room temperature. After 10 minutes the crude 2-methyl-6-piperidin-4-yl-2H-pyridazin-3-one solution from above was slowly added and the resulting mixture stirred at room temperature for 15 hours. The reaction mixture was then concentrated and the residue was subjected to reversed phase preparative HPLC (15→100% acetonitrile in 0.1 M aqueous ammonium acetate) to give 97 mg (71%) of the title product.

¹H NMR (400 MHz, dimethyl sulfoxide-d₆ as solvent and internal reference) 1.26-1.43 (m, 2H), 1.47-1.77 (m, 6H), 2.41-2.56 (m, 3 H), 2.61-2.76 (m, 2H), 2.96 (t, 1H, J=12.5 Hz), 3.54 (s, 3H), 3.63 (broad d, 2H, J=11.5 Hz), 3.94 (broad d, 1H, J=13.1 Hz), 4.39 (broad d, 1H, J=12.5 Hz), 6.84 (d, 1H, J=9.7 Hz), 6.94 (s, 1H), 7.27 (dd, 1H, J=2.0, 8.9 Hz), 7.40 (d, 1H, J=9.5 Hz), 7.45 (d, 1H, J=8.9 Hz), 7.74 (d, 1H, J=2.0 Hz), 11.88 (broad s, 1H).

HRMS (ESI+) calc. [M+H]⁺ 518.1623, found 518.1611.

### EXAMPLE 16

### i) 6-(4-{(S)-4-[(E)-2-(5-chloro-thiophen-2-yl)-ethenesulfonyl]-2-methyl-6-oxo-piperazin-1-ylmethyl}-piperidin-1-yl)-2-methyl-2H-pyridazin-3-one and

### ii) 6-(4-{(S)-4-[(E)-2-(5-chloro-thiophen-2-yl)-ethenesulfonyl]-5-methyl-2-oxo-piperazin-1-ylmethyl}-piperidin-1-yl)-2-methyl-2H-pyridazin-3-one

### A) i) ((S)-2-{1-(1-methyl-6-oxo-1,6-dihydro-pyridazin-3-yl)-piperidin-4-ylmethyl]-amino}-propyl)-carbamic acid tert-butyl ester and

### ii) ((S)-1-methyl-2-}[1-(1-methyl-6-oxo-1,6-dihydro-pyridazin-3-yl)-piperidin-4-ylmethyl]-amino}-ethyl)-carbamic acid tert-butyl ester

To a solution of ((S)-2-amino-propyl)-carbamic acid tert-butyl ester (1.27 g, 7.12 mmol) in anhydrous dichloromethane (37 mL) was added 1-(1-methyl-6-oxo-1,6-dihydro-pyridazin-3-yl)-piperidine-4-carbaldehyde (1.25 g, 5.65 mmol) under nitrogen. After stirring the resulting mixture at room temperature for 40 minutes, sodium triacetoxyborohydride (3.2 g, 15 mmol) was added and the mixture was stirred for 90 minutes. The reaction flask was cooled to 0° C., and the reaction mixture was quenched by adding water. Dichloromethane was evaporated under reduced pressure and the aqueous phase was freeze dried over night. The residue was suspended in dichloromethane, filtered and the solution was evaporated to dryness. The crude product was purified by column chromatography on silica gel using dichloromethane/methanol (100:10 and 100:20) as eluent to give 2.14 g (99%) of the sub-title products as a mixture. The mixture was used in the next step without separation of the two sub-title products.

### B) i) ((S)-2-{(2-chloro-acetyl)-[1-(1-methyl-6-oxo-1,6-dihydro-pyridazin-3-yl)-piperidin-4-ylmethyl]-amino}-propyl)-carbamic acid tert-butyl ester and

### ii) ((S)-2-{(2-chloro-acetyl)-[1-(1-methyl-6-oxo-1,6-dihydro-pyridazin-3-yl)-piperidin-4-ylmethyl]-amino}-1-methyl-ethyl)-carbamic acid tert-butyl ester

To a solution of the sub-title products from step A, i.e. i) ((S)-2-{[1-(1-methyl-6-oxo-1,6-dihydro-pyridazin-3-yl)-piperidin-4-ylmethyl]-amino}-propyl)-carbamic acid tert-butyl ester and ii) ((S)-1-methyl-2-{[1-(1-methyl-6-oxo-1,6-dihydro-pyridazin-3-yl)-piperidin-4-ylmethyl]-amino}-ethyl)-carbamic acid tert-butyl ester, (1.7 g, 4.8 mmol) in anhydrous dichloromethane (30 mL) was added triethylamine (1.7 mL, 12 mmol) at 0° C. dropwise under nitrogen. A solution of bromoacetyl chloride (0.66 g, 5.8 mmol) in anhydrous dichloromethane (7 mL) was added at 0° C. dropwise to the mixture, and the reaction mixture was stirred at room temperature for 1 hour. The reaction flask was cooled to 0° C., and water/dichloromethane was added. The organic phase was separated, washed with brine, dried and evaporated under reduced pressure. The crude product was purified by column chromatography on silica gel using dichloromethane/methanol (100:4) as eluent to give 0.91 g (42%) of the sub-title products as a mixture. The mixture was used in the next step without separation of the two the sub-title products.

### C) i) N-((S)-2-amino-1-methyl-ethyl)-2-chloro-N-[1-(1-methyl-6-oxo-1,6-dihydro-pyridazin-3-yl)-piperidin-4-ylmethyl]-acetamide hydrochloride and

### ii) N-((S)-2-amino-propyl)-2-chloro-N-[1-(1-methyl-6-oxo-1,6-dihydro-pyridazin-3-yl)-piperidin-4-ylmethyl]-acetamide hydrochloride

To a solution of the sub-title products from step B, i.e. i) ((S)-2-{(2-chloro-acetyl)-[1-(1-methyl-6-oxo-1,6-dihydro-pyridazin-3-yl)-piperidin-4-ylmethyl]-amino}-propyl)-carbamic acid tert-butyl ester and ii) ((S)-2-{(2-chloro-acetyl)-[1-(1-methyl-6-oxo-1,6-dihydro-pyridazin-3-yl)-piperidin-4-ylmethyl]-amino}-1-methyl-ethyl)-carbamic acid tert-butyl ester, (0.91 g, 1.99 mmol) in methanol (25 mL) was added a saturated methanolic hydrogen chloride (50 mL) at 0° C. After stirring at room temperature for 11 hour, the solution was evaporated to dryness to give 0.73 g (93%) of the sub-title products. The mixture of the sub-title products was used in the next step without separation of the two sub-title products.

### D) i) 2-Methyl-6-[4-((S)-2-methyl-6-oxo-piperazin-1-ylmethyl)-piperidin-1-yl]-2H-pyridazin-3-one and

### ii) 2-methyl-6-[4-((S)-5-methyl-2-oxo-piperazin-1-ylmethyl)-piperidin-1-yl]-2H-pyridazin-3-one

To a solution of the sub-title products from step C, i.e. i) N-((S)-2-amino-1-methyl-ethyl)-2-chloro-N-[1-(1-methyl-6-oxo-1,6-dihydro-pyridazin-3-yl)-piperidin-4-ylmethyl]-acetamide hydrochloride and ii) N-((S)-2-amino-propyl)-2-chloro-N-[1-(1-methyl-6-oxo-1,6-dihydro-pyridazin-3-yl)-piperidin-4-ylmethyl]-acetamide hydrochloride, (0.73 g, 1.86 mmol) in anhydrous N,N-dimethylformamide (9 mL) was added triethylamine (2 mL) at 0° C. under nitrogen. After stirring at room temperature for 8 hours, the solution evaporated to dryness and the crude product was purified by preparative HPLC using acetonitrile and ammonium acetate buffer (5:95 to 40:60) as eluent to give 30 mg (5%) of the sub-title products. The mixture of the sub-title products was used in the next step without separation of the two sub-title products.

E) To a solution of the sub-title products from step D, i.e. i) 2-methyl-6-[4-((S)-2-methyl-6-oxo-piperazin-1-ylmethyl)-piperidin-1-yl]-2H-pyridazin-3-one and ii) 2-methyl-6-[4-((S)-5-methyl-2-oxo-piperazin-1-ylmethyl)-piperidin-1-yl]-2H-pyridazin-3-one, (30 mg, 0.09 mmol) in anhydrous N,N-dimethylformamide (1 mL) was added triethylamine (30 mg, 0.3 mmol) and a solution of (E)-2-(5-chloro-thiophen-2-yl)-ethenesulfonyl chloride (25 mg, 0.1 mmol) in anhydrous dichloromethane (1 mL) under nitrogen. After stirring for 40 minutes, the reaction flask was cooled to 0° C. and the reaction mixture was quenched by adding water. The aqueous phase was extracted with dichloromethane, and then the organic phase dried and evaporated to dryness. The crude product was purified by column chromatography on silica gel using dichloromethane/methanol (100:4) as eluent to give 27 mg (55%) of the products of Example 16 as a mixture. The two products of Example 16 (17 mg) were separated by preparative HPLC using acetonitrile and ammonium acetate buffer (25:75 to 55:45) to give 5.3 mg of pure i) 6-(4-{(S)-4-[(E)-2-(5-chloro-thiophen-2-yl)-ethenesulfonyl]-2-methyl-6-oxo-piperazin-1-ylmethyl}-piperidin-1-yl)-2-methyl-2H-pyridazin-3-one and 12 mg of pure ii) 6-(4-{(S)-4-[(E)-2-(5-chloro-thiophen-2-yl)-ethenesulfonyl]-5-methyl-2-oxo-piperazin-1-ylmethyl}-piperidin-1-yl)-2-methyl-2H-pyridazin-3-one.

### Example 16, i)

¹H NMR (500 MHz, methanol-d₄ as solvent and internal reference) for Example 16, i) 1.20-1.32 (m, 2H), 1.33 (d, 3H, J=6.4 Hz), 1.63-1.77 (m, 2H), 1.91-2.01 (m, 1H), 2.67-2.79 (m, 2H), 2.81-2.88 (m, 1H), 3.27-3.30 (m, 1H), 3.50-3.62 (m, 1H), 3.63 (s, 3H), 3.70-3.76 (m, 2H), 3.79-3.86 (m, 1H), 3.87-3.98 (m, 3H), 6.81 (d, 1H, J=15.4 Hz), 6.84 (d, 1H, J=9.8 Hz), 7.04 (d, 1H, J=3.8 Hz), 7.32 (d, 1H, J=3.8 Hz), 7.45 (d, 1H, J=9.8 Hz), 7.57 (d, 1H, J=15.4 Hz).

HRMS (ESI+) for Example 16, i) calc. [M+H]⁺ 526.1344, found 526.1321.

### Example 16, ii)

¹H NMR (500 MHz, methanol-d₄ as solvent and internal reference) for Example 16, ii): 1.13-1.32 (m, 2H), 1.33 (d, 3H, J=6.6 Hz), 1.60-1.72 (m, 2H), 1.80-1.89 (m, 1H), 2.61-2.71 (m, 2H), 3.10-3.17 (m, 1H), 3.28 (dd, 1H, J=4.5, 13.2 Hz), 3.45-3.52 (m, 1H), 3.64 (s, 3H), 3.71 (dd, 1H, J=4.9, 13.2 Hz), 3.78-3.83 (m,1H), 3.85-3.90 (m, 1H), 3.92 (d, 1H, J=17.6Hz), 4.07 (d, 1H, J=17.6 Hz), 4.10-4.18 (m, 1H), 6.80 (d, 1H, J=15.3 Hz), 6.86 (d, 1H, J=10.0 Hz), 7.06 (d, 1H, J=3.9 Hz), 7.31 (d, 1H, J=3.9 Hz), 7.42 (d, 1H, J=10.0 Hz), 7.56 (d, 1H, J=15.3 Hz).

HRMS (ESI+) for Example 16, ii) calc. [M+H]⁺ 526.1344, found 526.1335.

### EXAMPLE 17

### (R)-4-(5-Chloro-1H-indole-2-sulfonyl)-1-[1-(1-methyl-6-oxo-1,6-dihydro-pyridazin-3-yl)-piperidin-4-ylmethyl]-6-oxo-piperazine-2-carboxylic acid methyl ester,

### A) (R)-3-tert-Butoxycarbonylamino-2-{[1-(1-methyl-6-oxo-1,6-dihydro-pyridazin-3-yl)-piperidin-4-ylmethyl]-amino}-propionic acid methyl ester

To a stirred mixture of (R)-2-amino-3-tert-butoxycarbonylamino-propionic acid methyl ester hydrochloride (1.90 g, 7.46 mmol) in dichloromethane (60 mL) was added triethyl-amine (1.13 mL, 8.14 mmol). The solvent was immediately removed in vacuo. The residue was dissolved in dichloromethane (60 mL) and the solvent was removed in vacuo again. To the residue was added dichloromethane (100 mL) and 1-(1-methyl-6-oxo-1,6-dihydro-pyridazin-3-yl)-piperidine-4-carbaldehyde (1.50 g, 6.78 mmol). The reaction mixture was stirred one hour and then sodium triacetoxyborohydride (2.87g, 13.6 mmol) was added in five portions. The reaction mixture was stirred two hours at room temperature and cooled on an ice bath before water (20 mL) was added dropwise. The dichloromethane was removed in vacuo. To the residue was added dimethyl sulfoxide (2 mL) before purification by preparative HPLC using a gradient of acetonitrile/5% acetonitrile-water phase containing 0.1 M ammonium acetate, to give 2.57 g (89%) of the sub-title product, after evaporation and freeze drying over night.

¹H NMR (400 MHz, dimethyl sulfoxide-d₆ as solvent and internal reference) δ(ppm) 1.05-1.20 (m, 2H), 1.35, 1.38 (s, s, 9H, rotamers), 1.43-1.54 (m, 1H), 1.68-1.76 (m, 2H), 2.23-2.30 (m, 1H), 2.36-2.42 (m, 1H), 2.58-2.68 (m, 2H), 3.05-3.18 (m, 2H), 3.20-3.26 (m, 1H), 3.47 (s, 3H), 3.59, 3.63 (s, s, 3H, rotamers), 3.77-3.84 (m,2H), 6.77 (d, 1H, J=10.0), 6.79-6.83 (m, 1H, NH), 7.47 (d, 1H, J=10.0).

### B) (R)-3-tert-Butoxycarbonylamino-2-{(2-chloro-acetyl)-[1-(1-methyl-6-oxo-1,6-dihydro-pyridazin-3-yl)-piperidin-4-ylmethyl]-amino}-propionic acid methyl ester

Was synthesized and purified essentially as in step C of example 5 using (R)-3-tert-butoxycarbonylamino-2-{[1-(1-methyl-6-oxo-1,6-dihydro-pyridazin-3-yl)-piperidin-4-ylmethyl]-amino}-propionic acid methyl ester (2.10 g, 4.96 mmol) from step A and chloro-acetyl chloride (0.84 g, 7.44 mmol) as starting materials to give 2.05 g (83%) of the sub-title compound.

¹H NMR (400 MHz, dimethyl sulfoxide-d₆ as solvent and internal reference) δ(ppm) 1.15-1.30 (m, 211), 1.37 (s, 9H), 1.44-1.81 (m, 3H), 2.58-2.72 (m, 2H), 3.02-3.11 (m, 1H), 3.33-3.56 (m, 3H), 3.47 (s, 3H), 3.58 (s, 3H), 3.77-3.96 (m, 3H), 4.30 (d, 1H, J=14.1 Hz), 4.41 (d, 1H, J=14.1 Hz), 6.79 (d, 1H, J=9.9 Hz), 6.92 (t, 1NH, J=5.8 Hz), 7.49 (d, 1H, J=9.9 Hz).

### C) (R)-3-Amino-2-{(2-chloro-acetyl)-[1-(1-methyl-6-oxo-1,6-dihydro-pyridazin-3-yl)-piperidin-4-ylmethyl]-amino}-propionic acid methyl ester hydrochloride

Was synthesized and purified essentially as in step D of example 5, but with a reaction time of 90 minutes, using (R)-3-tert-butoxycarbonylamino-2-{(2-chloro-acetyl)-[1-(1-methyl-6-oxo-1,6-dihydro-pyridazin-3-yl)-piperidin-4-ylmethyl]-amino}-propionic acid methyl ester (2.00 g, 4.00 mmol) from step B as starting material to give 1.71 g (98%) of the sub-title compound.

¹H NMR (400 MHz, methanol-d₄ as solvent and internal reference) δ(ppm) 1.27-1.46 (m, 2H), 1.78-2.04 (m, 3H), 2.80-2.92 (m, 2H), 3.31-3.39 (m, 2H), 3.46-3.54 (m, 1H), 3.58-3.65 (m, 1H), 3.73 (s, 3H), 3.77 (s, 3H), 4.06-4.14 (m, 2H), 4.28-4.41 (m, 3H), 7.03 (d, 1H, J=9.9), 7.63 (d, 1H, J=9.9).

### D) (R)-1-[1-(1-Methyl-6-oxo-1,6-dihydro-pyridazin-3-yl)-piperidin-4-ylmethyl]-6-oxo-piperazine-2-carboxylic acid methyl ester hydrochloride

Was synthesized and purified essentially as in step E of example 5, but with a reaction time of 30 minutes using (R)-3-amino-2-{(2-chloro-acetyl)-[1-(1-methyl-6-oxo-1,6-dihydro-pyridazin-3 -yl)-piperidin-4-ylmethyl]-amino}-propionic acid methyl ester hydrochloride (1.70 g, 4.25 mmol) from step C as starting material. After purification the solids were treated with 1.25 M hydrochloric acid in methanol and evaporated under reduced pressure to give 1.43 g, (84%) of the sub-title compound.

¹H NMR (400 MHz, dimethyl sulfoxide-d₆ as solvent and internal reference) δ(ppm) 1.09 (dq, 1H, J=3, 1 Hz), 1.19 (dq, 1H, J=3, 1 Hz), 1.65 (broad t, 2H, J=13.2 Hz), 1.75-1.86 (m, 1H), 2.58-2.68 (m, 2H), 2.72 (dd, 1H, J=7.1, 13.7 Hz), 3.47 (s, 3H), 3.60-3.85 (m, 7H), 3.74 (s, 3H), 4.63-4.68 (m, 1H), 6.78 (d, 1H, J=10.0 Hz), 7.49 (d, 1H, J=10.0), 9.65 (broad s, 1NH), 10.56 (broad s, 1NH).

### E) (R)-4-(1-Benzenesulfonyl-5-chloro-1H-indole-2-sulfonyl)-1-[1-(1-methyl-6-oxo-1,6-dihydro-pyridazin-3-yl)-piperidin-4-ylmethyl]-6-oxo-piperazine-2-carboxylic acid methyl ester

To a mixture of (R)-1-[1-(1-methyl-6-oxo-1,6-dihydro-pyridazin-3-yl)-piperidin-4-ylmethyl]-6-oxo-piperazine-2-carboxylic acid methyl ester hydrochloride (0.15 g, 0.38 mmol) from step D in anhydrous dichloromethane (3 mL) was added triethylamine (0.18 mL, 1.3 mmol) at 0° C. under nitrogen. To the mixture, a solution of 1-benzenesulfonyl-5-chloro-1H-indole-2-sulfonyl chloride (366 mg, 0.94 mmol) in anhydrous dichloromethane (2 mL) was added at 0° C., and the reaction mixture was stirred at room temperature for 30 minutes. Solvent was removed with evaporator in vacuo before purification by HPLC using a gradient of acetonitrile/5% acetonitrile-water phase containing 0.1 M ammonium acetate, to give 124 mg (46%) of the sub-title compound after evaporation and freeze drying over night.

¹H NMR (400 MHz, dimethyl sulfoxide-d₆ as solvent and internal reference) δ(ppm) 1.07-1.27 (m, 2H), 1.58-1.71 (m, 2H), 1.76-1.87 (m, 1H), 2.59-2.71 (m, 3H), 3.47 (s, 3H), 3.63 (s, 3H), 3.74-3.86 (m, 4H), 3.97-4.12 (m, 3H), 4.48-4.51 (m, 1H), 6.78 (d, 1H, J=10.0 Hz), 7.48 (d, 1H, J=10.0 Hz), 7.57-7.63 (m, 3H), 7.68-7.74 (m, 2H), 7.78 (d, 1H, J=2.1 Hz), 7.97-8.02 (m, 2H), 8.19 (d, 1H, J=9.1 Hz).

### F)

A mixture of (R)-4-(1-benzenesulfonyl-5-chloro-1H-indole-2-sulfonyl)-1-[1-(1-methyl-6-oxo-1,6-dihydro-pyridazin-3-yl)-piperidin-4-ylmethyl]-6-oxo-piperazine-2-carboxylic acid methyl ester (0.15 g, 0.21 mmol) from step E and tetrabutylammonium fluoride (0.23 mL 1.0 M solution in tetrahydrofuran, 0.23 mmol) in tetrahydrofuran (1.5 mL) were treated with microwave at 100° C. for 5 minutes. The solution was concentrated in vacuo, and the residue was triturated with water. The crude product was purified by HPLC using a gradient of acetonitrile/5% acetonitrile-water phase containing 0.1 M ammonium acetate, to give 110 mg (91%) of the title product, after evaporation and freeze drying over night.

¹H NMR (500 MHz, dimethyl sulfoxide-d₆ as solvent and internal reference) δ(ppm) 1.01 (dq, 1H, J=4, 12 Hz), 1.13 (dq, 1H, J=4, 12 Hz), 1.48 (broad d, 1H, J=12 Hz), 1.58 (broad d, 1H, J=12 Hz), 1.67-1.76 (m, 1H), 2.48-2.64 (m, 3H), 3.15 (dd, 1H, J=3.4 12.3 Hz), 3.44 (s, 3H), 3.54 (d, 1H, J=16.2 Hz), 3.67 (s, 3H), 3.68-3.78 (m, 3H), 3.86 (d, 1H, J=16.2 Hz), 4.01 (d, 1H, J=12.3 Hz), 4.45 (t, 1H, J=2.6 Hz), 6.75 (d, 1H, J=10.0 Hz), 7.07 (s, 1H), 7.30 (dd, 1H, J=2.0, 8.8 Hz), 7.42 (d, 1H, J=10.0 Hz), 7.48 (d, 1H, J=8.8 Hz), 7.77 (d, 1H, 2.0 Hz).

HRMS (ESI+) calc. [M+H]⁺ 577.1630, found 577.1626.

### EXAMPLE 18

### (R)-4-(5-Chloro-1H-indole-2-sulfonyl)-1-[1-(1-methyl-6-oxo-1,6-dihydro-pyridazin-3-yl)-piperidin-4-ylmethyl]-6-oxo-piperazine-2-carboxylic acid

To a solution of the product from Example 17, i.e. (R)-4-(5-chloro-1H-indole-2-sulfonyl)-1-[1-(1-methyl-6-oxo-1,6-dihydro-pyridazin-3-yl)-piperidin-4-ylmethyl]-6-oxo-piperazine-2-carboxylic acid methyl ester, (55 mg, 0.095 mmol) in tetrahydrofuran (0.75 mL) was added a solution of lithium hydroxide (7 mg, 0.29 mmol) in water (0.25 mL) at room temperature. The reaction mixture was stirred for 1 hour before acetic acid was added to neutralize the reaction mixture. The crude product was purified by HPLC using a gradient of acetonitrile/5% acetonitrile-water phase containing 0.1 M ammonium acetate, to give 50 mg (93%) of the title compoundafter evaporation and freeze drying over night.

¹H NMR (500 MHz, dimethyl sulfoxide-d₆ as solvent and internal reference) δ(ppm) 0.95 (dq, 1H, J=4, 12 Hz), 1.07 (dq, 1H, J=4, 12 Hz), 1.35 (broad d, 1H, J=12 Hz), 1.52 (broad d, 1H, J=12 Hz), 1.61-1.70 (m, 1H), 2.48-2.64 (m, 3H), 3.16 (dd, 1H, J=4.0 11.7 Hz), 3.45 (s, 3H), 3.52-3.79 (m, 6H), 3.96 (dd, 1H, J=3.3, 11.7 Hz), 6.75 (d, 1H, J=10.0 Hz), 7.04 (s, 1H), 7.31 (dd, 1H, J=2.0, 8.8 Hz), 7.39 (d, 1H, J=10.0 Hz), 74.7 (d, 1H, J=8.8 Hz), 7.76 (d, 1H, J=2.0 Hz).

HRMS (ESI+) calc. [M+H]⁺ 563.1474, found 563.1449.

### EXAMPLE 19

### (R)-4-(6-Chloro-naphthalene-2-sulfonyl)-1-[1-(1-methyl-6-oxo-1,6-dihydro-pyridazin-3-yl)-piperidin-4-ylmethyl]-6-oxo-piperazine-2-carboxylic acid methyl ester

Was synthesized and purified essentially as in example 2 using 6-chloro-naphthalene-2-sulfonyl chloride (197 mg, 0.75 mmol) as starting material to give 271 mg (73%) of the title compound.

¹H NMR (500 MHz, dimethyl sulfoxide-d₆ as solvent and internal reference) δ(ppm) 1.00 (dq, 1H, J=4, 12 Hz), 1.12 (dq, 1H, J=4, 12 Hz), 1.46 (broad d, 1H, J=12 Hz), 1.56 (broad d, 1H, J=12 Hz), 1.65-1.75 (m, 1H), 2.48-2.64 (m, 3H), 3.14 (dd, 1H, J=3, 12 Hz), 3.43 (s, 3H), 3.46 (d, 1H J=16 Hz), 3.67 (s, 3H), 3.67-3.77 (m, 3H), 3.88 (d, 1H, J=16 Hz), 4.06 (broad d, 1H, J=12 Hz), 4.42 (t, 1H, J=3 Hz), 6.75 (d, 1H, J=10 Hz), 7.41 (d, 1H, J=10 Hz), 7.73 (dd, 1H, J=2, 9 Hz), 7.85 dd, 1H, J=2, 9 Hz), 8.16 (d, 1H, J=9 Hz), 8.25 (d, 1H, J=2 Hz), 8.28 (d, 1H, J=9 Hz), 8.58 (broad s, 1H).

HRMS (ESI+) calc. [M+H]⁺ 588.1678, found 588.1664.

### EXAMPLE 20

### (R)-4-(6-Chloro-naphthalene-2-sulfonyl)-1-[1-(1-methyl-6-oxo-1,6-dihydro -pyridazin-3-yl)-piperidin-4-ylmethyl]-6-oxo-piperazine-2-carboxylic acid

The title product was synthesized and purified essentially as in example 18, but with a reaction time of 15 minutes using (R)-4-(6-chloro-naphthalene-2-sulfonyl)-1-[1-(1-methyl-6-oxo-1,6-dihydro-pyridazin-3-yl)-piperidin-4-ylmethyl]-6-oxo-piperazine-2-carboxylic acid methyl ester (103 mg, 0.18 mmol) as starting material to give 93 mg (92%) of the title compound.

¹H NMR (500 MHz, dimethyl sulfoxide-d₆ as solvent and internal reference) δ(ppm) 0.86 (dq, 1H, J=4, 12 Hz), 1.01 (dq, 1H, J=4, 12 Hz), 1.21 (broad d, 1H, J=12 Hz), 1.43 (broad d, 1H, J=12 Hz), 1.51-1.61 (m, 1H), 2.34-2.54 (m, 3H), 3.08 (dd, 1H, J=4.5, 11.5), 3.45 (s, 3H), 3.46-3.56 (m, 2H), 3.61-3.69 (m, 4H), 3.83-3.89 (m, 1H), 6.75 (d, 1H, J=10.0 Hz), 7.36 (d, 1H, J=10.0 Hz), 7.71 (dd, 1H, J=2.2, 8.8 Hz), 7.83 (dd, 1H, J=1.8, 8.7 Hz), 8.14 (d, 1H, J=8.8 z), 8.23 (d, 1H, J=1.9 Hz), 8.27 (d, 1H, J=8.9 Hz), 8.54 (broad s, 1H).

HRMS (ESI+) calc. [M+H]⁺ 574.1521, found 574.1533.

### EXAMPLE 21

### (R)-4-[(E)-2-(5-Chloro-thiophen-2-yl)-ethenesulfonyl]-1-[1-(1-methyl-6-oxo-1,6-dihydro-pyridazin-3-yl)-piperidin-4-ylmethyl]-6-oxo-piperazine-2-carboxylic acid

The title product was synthesized and purified essentially as in example 20, using (R)-4-[(E)-2-(5-chloro-thiophen-2-yl)-ethenesulfonyl]-1-[1-(1-methyl-6-oxo-1,6-dihydro-pyridazin-3-yl)-piperidin-4-ylmethyl]-6-oxo-piperazine-2-carboxylic acid methyl ester (170 mg, 0.30 mmol) as starting material to give 153 mg (92%) of the title compound.

¹H NMR (500 MHz, dimethyl sulfoxide-d₆ as solvent and internal reference) δ(ppm) 1.06 (dq, 1H, J=4, 12 Hz), 1.15 (dq, 1H, J=4, 12 Hz), 1.54 (broad d, 1H, J=12 Hz), 1.63 (broad d, 1H, J=12 Hz), 1.70-1.81 (m, 1H), 2.52-2.64 (m, 3H), 3.23-3.32 (m, 1H), 3.46 (s, 3H), 3.60 (d, 1H, J=16.3 Hz), 3.66 (d, 1H, J=16.3 Hz), 3.70-3.86 (m, 5H), 6.77 (d, 1H, J=10.0 Hz), 6.97 (d, 1H, J=15.3 Hz), 7.19 (d, 1H, J=4.0 Hz), 7.44 (d, 1H, J=10.0 Hz), 7.49 (d, 1H, J=4.0 Hz), 7.52 (d, 1H, J=15.3 Hz).

HRMS (ESI+) calc. [M+H]⁺ 556.1086, found 556.1091.

### EXAMPLE 22

### (R)-4-[(E)-2-(5-Chloro-thiophen-2-yl)-ethenesulfonyl]-1-[1-(1-methyl-6-oxo-1,6-dihydro-pyridazin-3-yl)-piperidin-4-ylmethyl]-6-oxo-piperazine-2-carboxylic acid methyl ester

The title product was synthesized and purified essentially as in example 2 using (E)-2-(5-chloro-thiophen-2-yl)-ethenesulfonyl chloride (146 mg, 0.60 mmol) as starting material to give 232 mg (81%) of the title compound.

¹H NMR (500 MHz, dimethyl sulfoxide-d₆ as solvent and internal reference) δ(ppm) 1.09 (dq,1H, J=4, 12 Hz), 1.19 (dq, 1H, J=4, 12 Hz), 1.56 (broad d, 1H, J=12 Hz), 1.64 (broad d, 1H, J=12 Hz), 1.74-1.84 (m, 1H), 2.57-2.67 (m, 3H), 3.37 (dd, 1H, J=3.4, 12.4 Hz), 3.46 (s, 3H), 3.69 (s, 3H), 3.73-3.83 (m, 5H), 3.86 (broad d, 1H, J=12.3 Hz), 4.48 (t, 1H, J=2.8 Hz), 6.77 (d, 1H, J=10.0 Hz), 6.98 (d, 1H, J=15.3 Hz), 7.22 (d, 1H, J=4.0 Hz), 7.46 (d, 1H, J=10.0 Hz), 7.51 (d, 1H, J=4.0 Hz), 7.59 (d, 1H, J=15.3 Hz).

HRMS (ESI+) calc. [M+H]⁺ 570.1242, found 570.1250.

### EXAMPLE 23

### 6-{4-[4-(6-Chloro-naphthalene-2-sulfonyl)-piperazine-1-carbonyl]-piperidin-1-yl}-2-methyl-2H-pyridazin-3-one

The title product was synthesized and purified essentially as in step B of Example 11 but with a reaction temperature of 180° C. and a reaction time of 20 hours using [4-(6-chloro-naphthalene-2-sulfonyl)-piperazin-1-yl]-piperidin-4-yl-methanone (0.15 g, 0.36 mmol, WO 96/10022) and 6-chloro-2-methyl-2H-pyridazin-3-one (77 mg, 0.53 mmol) as starting materials to give 78 mg (41%) of the title compound.

¹H NMR (400 MHz, dimethyl sulfoxide-d₆ as solvent and internal reference) δ(ppm) 1.39-1.57 (m, 4H), 2.63-2.75 (m, 3H), 2.92-2.98 (m, 4H), 3.44 (s, 3H), 3.50-3.63 (m, 4H), 3.73-3.80 (m, 2H), 6.76 (d, 1H, J=10.0 Hz), 7.43 (d, 1H, J=10.0 Hz), 7.72 (dd, 1H, J=2.1, 8.8 Hz), 7.80 (dd, 1H, J=1.7, 8.8 Hz), 8.16 (d, 1H, J=8.8 Hz), 8.24 (d, 1H, J=1.8 Hz), 8.26 (d, 1H, J=8.8 Hz), 8.49 (s, 1H).

HRMS (ESI+) calc. [M+H]⁺ 530.1623, found 530.1624.

### EXAMPLE 24

### 6-(4-{4-[(E)-2-(5-Chloro-thiophen-2-yl)-ethenesulfonyl]-2-oxo-piperazin-1-ylmethyl}-piperidin-1-yl)-2-methyl-2H-pyridazin-3-one

To a solution of 2-methyl-6-[4-(2-oxo-piperazin-1-ylmethyl)-piperidin-1-yl]-2H-pyridazin-3-one (43 mg, 0.14 mmol) in anhydrous N,N-dimethylformamide (1 mL) was added triethylamine (57 mg, 0.56 mmol) and a solution of (E)-2-(5-Chloro-thiophen-2-yl)-ethenesulfonyl chloride (38 mg, 0.16 mmol) in anhydrous dichloromethane (1 mL) under nitrogen. After stirring for 140 minutes, the reaction flask was cooled to 0° C. and the reaction mixture was quenched by adding water. The aqueous phase was extracted with dichloromethane, and then the organic phase dried and evaporated to dryness. The crude product was purified by column chromatography on silica gel using dichloromethane/methanol (100:4) as eluent to give 25 mg (35%) of the title compound.

¹H NMR (500 MHz, methanol-d₄ as solvent and internal reference): 1.25-1.36 (m, 2H), 1.66-1.74 (m, 2H), 1.88-2.10 (m, 1H), 2.68-2.77 (m, 2H), 3.32-3.39 (m, 3H), 3.55 (broad s, 3H), 3.65 (s, 3H), 3.87-3.95 (m, 4H), 6.85 (d, 1H, J=15.0 Hz), 6.87 (d, 1H, J=10.1 Hz), 7.07 (d, 1H, J=3.9 Hz), 7.34 (d, 1H, J=3.9 Hz), 7.46 (d, 1H, J=9.8 Hz), 7.60 (d, 1H, J=15.3Hz).

HRMS (ESI+) calc. [M+H]⁺ 512.1187, found 512.1183.

### EXAMPLE 25

### 6-{4-[4-(5-Chloro-1H-indole-2-sulfonyl)-piperazine-1-carbonyl]-piperidin-1-yl}-2-methyl-2H-pyridazin-3-one

1-(1-methyl-6-oxo-1,6-dihydro-pyridazin-3-yl)-piperidine-4-carboxylic acid (0.15 g, 0.55 mmol), 5-chloro-2-(piperazine-1-sulfonyl)-1H-indole (0.16 g, 0.55 mmol) and 4-dimethyl aminopyridine (0.23 g, 1.9 mmol) was dissolved in N,N-dimethylformamide (4 mL) before o-(benzotriazol-1yl)-N,N,N′,N′-tetramethyluronium tetrafluoroborate (0.35 g, 1.1 mmol) was added. The reaction mixture was stirred under nitrogen atmosphere at room temperature for 30 minutes. The crude product was purified by HPLC using a gradient of acetonitrile/5% acetonitrile water phase containing 0.1 M ammonium acetate, to give to give 133 mg (47%) of the title compound.

¹H NMR (400 MHz, dimethyl sulfoxide-d₆ as solvent and internal reference) δ(ppm) 1.41-1.59 (m, 4H), 2.64-2.77 (m, 3H), 2.96-3.02 (m, 4H), 3.45 (s, 3H), 3.51-3.62 (m, 4H), 3.75-3.81 (m, 2H), 6.76 (d, 1H, J=10.0Hz), 7.00 (s, 1H), 7.30 (dd, 1H, J=2.0, 8.9 Hz), 7.42-7.49 (m, 3H), 7.76 (d, 1H, J=1.9 Hz).

HRMS (ESI+) calc. [M+H]⁺ 519.1576, found 519.1567.

### EXAMPLE 26

### 6-(4-{4-[(E)-2-(5-Chloro-thiophen-2-yl)-ethenesulfonyl]-piperazine-1-carbonyl}-piperidin-1-yl)-2-methyl-2H-pyridazin-3-one

### A) 1-[(E)-2-(5-Chloro-thiophen-2-yl)-ethenesulfonyl]-piperazine

Piperazine (7.08 g, 82.3 mmol) was dissolved in 5 mL N,N-dimethylformamide and cooled to 0° C. (E)-2-(5-chloro-thiophen-2-yl)-ethenesulfonyl chloride (1.00 g, 4.11 mmol) was added at 0° C. and the solution was stirred at room temperature for 30 minutes. The solvent was removed by evaporation in vacuo and the crude residue was purified by preparative HPLC (starting with isocratic acetonitrile/buffer 30/70 and then the acetonitrile concentration was increased to 100%, the buffer was a mixture of acetonitrile/water 10/90 and ammonium acetate (0.1 M, column KR-100-7-C8, 50 mm×250 mm, flow 40 mL/min). The product containing fractions was pooled and the acetonitrile was removed by evaporation and the sub-title product was obtained after freeze drying over night in 548 mg (45%) yield).

¹H NMR (500MHz, chloroform-d as solvent and internal reference) δ(ppm) 3.02 (m, 4H), 3.23 (m, 4H), 6.32 (d, 1H, J=15.3 Hz), 6.88 (d, 1H, J=4.0 Hz), 7.05 (d, 1H, J=4.0 Hz), 7.40 (d, 1H, J=15.3 Hz).

### B)

1-(1-methyl-6-oxo-1,6-dihydro-pyridazin-3-yl)-piperidine-4-carboxylic acid (60 mg, 0.25 mmol), 1-[(E)-2-(5-Chloro-thiophen-2-yl)-ethenesulfonyl]-piperazine (84 mg, 0.29 mmol), 4-dimethylaminopyridine (120 mg, 0.98 mmol), 1-ethyl-3-(3-dimethyl-aminopropyl) carbodiimide hydrochloride (106 mmol, 0.55 mmol) and 1.5 mL N,N-dimethylformamide were stirred for 17 hours at room temperature. Ethyl acetate and water was added, the phases were separated. The organic phase was washed with 1 M hydrochloric acid, 1M sodium hydrogen carbonate, water and brine, dried over magnesium sulfate and evaporated in vacuo. The crude was further purified by preparative HPLC using a gradient of CH₃CN/5% CH₃CN in 0.1 M ammonium acetate water buffer, to give 84 mg (64% yield) of the title product as a yellow solid after evaporation of solvent in vacuo.

¹H NMR (400 MHz, chloroform-d as solvent and internal reference) δ(ppm) 1.75 (m, 2H), 1.88 (m, 2H), 2.62 (m, 1H), 2.77 (m, 2H), 3.20 (m, 4H), 3.63-3.76 (m, 7H), 3.84 (m, 2H), 6.30 (d, 1H, J=15.1Hz), 6.84 (d, 1H, J=10.2 Hz), 6.91 (d, 1H, J=3.8 Hz), 7.07-7.11 (m, 2H), 7.44 (d, 1H, J=15.1 Hz).

HRMS (ESI+) calc. [M+H]⁺ 512.1188, found 512.1210.

### EXAMPLE 27

### 6-{4-[4-(5-Chloro-1H-indole-2-sulfonyl)-3-hydroxy-piperazine-1-carbonyl]-piperidin-1-yl}-2-methyl-2H-pyridazin-3-one

### A) 1-(1-methyl-6-oxo-1,6-dihydro-pyridazin-3-yl)-piperidine-4-carboxylic acid Allyl-[2-(1-benzenesulfonyl-5-chloro-1H-indole-2-sulfonylamino)-ethyl]-amide

A suspension of 1-benzenesulfonyl-5-chloro-1H-indole-2-sulfonyl chloride (320 mg, 390 μmol) in 1 mL dry dichloromethane was added to a stirred solution of 1-(1-methyl-6-oxo-1,6-dihydro-pyridazin-3-yl)-piperidine-4-carboxylic acid allyl-(2-amino-ethyl)-amide (203 mg, 319 μmol) and N,N-diisopropylethylamine (0.44 mL, 2.5 mmol) in 3.5 mL dry dichloromethane. The reaction mixture was stirred at room temperature for 2.5 hours under nitrogen and then diluted with dichloromethane. Water was added and the aqueous layer was adjusted to ˜pH 5 using 1 M aqueous potassium hydrogen sulfate. The phases were separated and the water phase was extracted with dichloromethane. The organic phases were pooled washed with brine, dried over sodium sulfate, filtered and concentrated in vacuo. The crude was further purified by preparative HPLC using a gradient of acetonitrile/5% acetonitrile in water buffer containing 0.1 M ammonium acetate to give 243 mg (56% yield) of the sub-title product as a yellow powder.

¹H NMR (400 MHz; chloroform-d as solvent and internal reference) δ(ppm) 1.74-1.97 (m, 4H), 2.59 (m, 1H), 2.78 (m, 2H), 3.22 (m, 2H), 3.53 (m, 2H), 3.63 (s, 3H), 3.84 )broad d, 2H, J=13.8 Hz), 3.96 (m, 0.4H, rotamer), 4.04 (m, 1.6H, rotamer), 5.15 (m, 2H), 5.79 (m, 1H), 6.03 (m, 1NH), 6.87 (d, 1H, J=9.7 Hz), 7.11 (d, 1H, J=9.7 Hz), 7.36-7.51 (m, 4H), 7.58 (m, 2H), 8.09 (m, 3H).

### B) 6-{4-[4-(1-Benzenesulfonyl-5-chloro-1H-indole-2-sulfonyl)-3-hydroxy-piperazine-1-carbonyl]-piperidin-1-yl}-2-methyl-2H-pyridazin-3-one

To a solution of 1-(1-methyl-6-oxo-1,6-dihydro-pyridazin-3-yl)-piperidine-4-carboxylic acid allyl-[2-(1-benzenesulfonyl-5-chloro-1H-indole-2-sulfonylamino)-ethyl]-amide (57 mg, 0.080 mmol) from step A in 1.5 mL tetrahydrofuran was added sodium periodate (61 mg, 0.29 mmol) dissolved in 0.5 mL water followed osmium tetroxide (2.5% wt solution in t-BuOH, 0.020 mL, 0.0016 mmol). The mixture was stirred over night at room temperature. Dichloromethane and water was added. The phases were separated and the water phase was extracted twice with dichloromethane. The organic phases were pooled, dried over sodium sulfate, filtered and evaporated in vacuo. The crude was further purified by preparative hplc using acetonitrile/5% acetonitrile in water buffer containing 0.1 M ammonium acetate to give 30 mg (52% yield) of the sub-title product after removal of solvents.

¹H NMR (500 MHz; acetonitrile-d₃ as solvent and internal reference) δ(ppm) 1.65-1.95 (m, 4H), 2.81 (m, 3H), 3.0 (broad d, 0.3H, J=12.2 Hz, rotamer), 3.29 (m, 0.4H, rotamer), 3.39-3.63 (m, 5.5H, rotamers), 3.88 (m, 2H), 4.10 (m, 1H), 4.35 (m, 0.3H, rotamer), 4.59 (m, 1.5H, rotamer), 5.63 (m, 1H), 6.76 (d, 1H, J=9.5 Hz), 7.29 (d,1H, J=9.5 Hz), 7.57 (m, 4H), 7.72 (m, 2H), 8.08 (m, 2H), 8.23 (d, 1H, J=9.2 Hz).

### C) 6-{4-[4-(5-Chloro-1H-indole-2-sulfonyl)-3-hydroxy-piperazine-1-carbonyl]-piperidin-1-yl}-2-methyl-2H-pyridazin-3-one

6-{4-[4-(1-Benzenesulfonyl-5-chloro-1H-indole-2-sulfonyl)-3-hydroxy-piperazine-1-carbonyl]-piperidin-1-yl}-2-methyl-2H-pyridazin-3-one (26 mg, 0.039 mmol) from step B, 3 mL tetrahydrofuran and tetrabutylammonium fluoride (0.046 mL, 0.046 mmol, 1M in tetrahydrofuran) was added to a vial for microwave irradiation. The reaction was run in a microwave oven at 100° C. for 3 minutes. Another equivalent of tetrabutylammonium fluoride was added and the reaction was run at 100° C. for 5 minutes, still starting material left. Another eqvivalent of tetrabutylammonium fluoride was added and the reaction was run for 5 minutes at 100° C. The solvent was evaporated, ethyl acetate and water was added, the phases were separated and the organic phase was washed four times with water. The organic phase was dried over sodium sulfate filtered and evaporated. The crude was further purified by preparative HPLC using a gradient of acetonitrile/5% acetonitrile in water buffer containing 0.1 M ammonium acetate to give 10 mg (48% yield) of the title product after concentration and freeze drying over night.

¹H NMR (600 MHz; dimethyl sulfoxide-d₆ as solvent and internal reference) The spectrum shows two sets of signals, major 70% and minor 30%, due to hindered rotation around the amide bonde. δ(ppm) 1.48-1.69 (m, 3H), 1.83 (m, 1H), 2.62 (m, 1H), 2.71 (m, 2H), 2.78 (m, 3H), 3.19 (m, 2H, minor), 3.36 (m, 1H, under solvent peak), 3.47 (m, 3H, minor), 3.50 (s, 3H), 3.86 (m, 2H), 3.99-4.12 (m, 1H), 4.37-4.51 (m, 1H), 5.47 (broad s, 1H), 6.70 (m, 1H), 6.80 (m, 1H), 6.90 (m, 1H), 7.29 (m, 1H), 7.48 (m, 2H).

HRMS (ESI+) calc. [M+H]⁺ 535.1530, found 535.1489.

### EXAMPLE 28

### 6-{4-[4-(3-Chloro-1H-indole-6-sulfonyl)-3,4-dihydro-2H-pyrazine-1-carbonyl]piperidin-1-yl}-2-methyl-2H-pyridazin-3-one

To 6-{4-[4-(3-chloro-1H-indole-6-sulfonyl)-3-hydroxy-piperazine-1-carbonyl]-piperidin-1-yl}-2-methyl-2H-pyridazin-3-one (10 mg, 0.020 mmol) dissolved in 2 mL methanol was added one drop of concentrated hydrochloric acid. The reaction was run for 1 hour at room temperature. The mixture was concentrated in vacuo to give 9 mg (93% yield) of the title product.

¹H NMR (400 MHz; acetonitrile-d₃ as solvent and internal reference) δ(ppm) 1.63-1.81 (m, 4H), 2.75 (m, 1H), 3.02 (m, 2H), 3.35 (m, 1H), 3.42 (m, 1H), 3.52 (m, 1H), 3.54 (m, 1H), 3.74 (s, 3H), 3.92 (broad d, 2H, J=13.1 Hz), 6.12 (d, 0.7H, J=6.7 Hz, rotamer), 6.23 (d, 0.3H, J=6.7 Hz, rotamer), 6.35 (d, 0.7H, J=6.9 Hz, rotamer), 6.66 (d, 0.3H, J=6.9 Hz, rotamer), 7.51-7.58 (m, 2H), 7.63 (m, 2H), 7.73 (broad d, 1H, J=9.3 Hz), 7.98 (s, 1H).

HRMS (ESI+) calc. [M+H]⁺ 517.1425, found 517.1441.

### EXAMPLE 29

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid dimethylamide

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid, i.e. the title product of Example 1, (50 mg, 0.09 mmol), 2-(7-aza-1H-benzotriazole-1-yl)-1,1,3,3-tetramethyluronium hexafluorophosphate (37 mg, 0.10 mmol) and dimethylamine hydrochloride (22 mg, 0.27 mmol) was dissolved in 2 mL dry N,N-dimethylformamide before N,N-diisopropylethylamine (0.077 mL, 0.44 mmol) was added. The reaction mixture was stirred over night at room temperature. Additional N,N-diisopropylethylamine (1 eq.), dimethylamine hydrochloride (1 eq and 2-(7-aza-1H-benzotriazole-1-yl)-1,1,3,3-tetramethyluronium hexafluorophosphate (1 eq) was added followed by benzotriazol 1-yl-oxytri-pyrrolidinophosphonium hexafluorophosphate (46 mg, 0.090 mmol). After 2 hours the mixture was purified by preparative HPLC using a gradient of acetonitrile/5% acetonitrile in water buffer containing 0.1 M ammonium acetate to give the product and a by-product from benzotriazol 1-yl-oxytri-pyrrolidinophosphonium hexafluorophosphate. The crude was dissolved in ethyl acetate and washed three times with 1 M hydrochloric acid and once with water, dried over sodium sulfate, filtered and evaporated in vacuo to give 7.5 mg (14% yield) of the title product as a white powder.

1H NMR (400 MHz, methanol-d₄ as solvent and internal reference) δ(ppm) 1.18 (m, 2H), 1.52 (broad d, 1H, J=13.0 Hz), 1.65 (broad d, 1H, J=13.0 Hz), 1.73 (m, 1H), 2.49 (m, 1H), 2.63 (m, 2H), 2.85 (s, 3H), 3.06 (s, 3H), 3.16 (m, 1H), 3.49 (d, 1H, J=16.7 Hz), 3.58 (s, 3H), 3.72 (m, 1H), 3.78-3.93 (m, 3H), 4.01 (d, 1H, J=16.7 Hz), 4.66 (m, 1H), 6.79 (d, 1H, J=10.0 Hz), 7.38 (d, 1H, J=10.0 Hz), 7.51 (m, 1H), 7.57 (s, 1H), 7.73 (d, 1H, J=8.3 Hz), 7.9 (s, 1H).

HRMS (ESI+) calc. [M+H]⁺ 590.1953, found 590.1965.

### EXAMPLE 30

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid ethylamide

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid, i.e. the title product of Example 1, (53 mg, 0.09 mmol), triethylamine (0.06 mL, 0.44 mmol) and ethylamine hydrochloride (14 mg, 0.18 mmol) was dissolved in 1.8 mL dry N,N-dimethylformamide. Benzotriazol-1-yl-oxytri-pyrrolidinophosphonium hexafluorophosphate (69 mg, 0.13 mmol) was added in one portion. The reaction was stirred for two hours at room temperature. The mixture was purified by preparative HPLC using a gradient of acetonitrile/5% acetonitrile in water buffer containing 0.1 M ammonium acetate to give the product and a by-product from benzotriazole-1-yl-oxy-tris-pyrrolidino-phosphonium hexafluorophosphate. The crude was further purified by flash chromatography on silica gel using dichloromethane/methanol (95:5) as eluent to give the product containing a small amount of byproduct. The crude was dissolved in ethyl acetate and washed with 1 M hydrochloric acid and water, dried over sodium sulfate, filtered and evaporated in vacuo to give pure title product, 25 mg, (45% yield) as a white powder.

¹H NMR (400 MHz, methanol-d₄ as solvent and internal reference) δ(ppm) 1.11 (t, 3H, J=7.2 Hz), 1.1-1.3 (m, 2H), 1.49 (broad d, 1H, J=13.3 Hz), 1.61 (broad d, 1H, J=13.3 Hz), 1.75 (m, 1H), 2.49-2.66 (m, 3H), 3.13 (m, 1H), 3.20 (q, 2H, J=7.2 Hz), 3.46 (d, 1H, J=16.1 Hz), 3.57 (s, 3H), 3.76-3.93 (m, 4H), 4.0 (d, 1H, J=16.1 Hz), 4.09 (m, 1H), 6.79 (d, 1H, J=9.3 Hz), 7.38 (d, 1H, J=9.3 Hz), 7.51 (m, 1H), 7.57 (s, 1H), 7.73 (d, 1H, J=8.6 Hz), 7.90 (s, 1H).

HRMS (ESI+) calc. [M+H]⁺ 590.1953, found 590.1959.

### EXAMPLE 31

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-hydroxy-ethyl)-amide

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid, i.e. the title product of Example 1, (50 mg, 0.090 mmol), triethylamine (0.10 mL, 0.72 mmol) and ethanol amine (11 mg, 0.18 mmol) was dissolved in 1.8 mL dry N,N-dimethylformamide. Benzotriazol-1-yl-oxytri-pyrrolidinophosphonium hexafluorophosphate (69 mg, 0.13 mmol) was added in one portion. The reaction was stirred over night at room temperature. The mixture was purified by preparative HPLC using a gradient of acetonitrile/5% acetonitrile in water buffer containing 0.1 M ammonium acetate to give 42 mg (78% yield) of the desired title product after freeze drying over night.

¹H NMR (300 MHz, acetic acid-d₄ as solvent and internal reference) δ(ppm) 1.24 (m, 2H), 1.48-1.68 (m, 2H), 1.89 (m, 1H), 2.67 (m, 3H), 3.12 (m, 1H), 3.49 (t, 2H, J=5.2 Hz), 3.58 (d, 1H, J=16.7 Hz), 3.66 (s, 3H), 3.79 (t, 2H, J=5.2 Hz), 3.84-4.0 (m, 3H), 4.10 (m, 1H), 4.19 (d, 1H, J=16.7 Hz), 4.36 (m, 1H), 7.10 (d, 1H, J=9.4 Hz), 7.34 (d, 1H, J=9.4 Hz), 7.55 (m, 2H), 7.75 (d, 1H, J=7.7 Hz), 7.99 (m, 1H).

HRMS (ESI+) calc. [M+H]⁺ 606.1901, found 606.193.

### EXAMPLE 32

### 6-{4-[4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one

### i) 6-{4-[(R)-4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one and ii) 6-{4-[(S)-4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid, i.e. the title product of Example 1, (78 mg, 0.14 mmol) and morpholine (0.050 mL, 0.57 mmol) was dissolved in 1.5 mL dry N,N-dimethylformamide, 2-(1H-benzotriazole-1-yl)-1,1,3,3-tetramethyluronium tetra-fluoroborate (54 mg, 0.17 mmol) was added in one portion. The reaction was stirred for 4 hours at room temperature. More 2-(1H-benzotriazole-1-yl)-1,1,3,3-tetramethyluronium tetrafluoroborate (25 mg, 0.080 mmol) was added and the mixture was stirred for 1 hour. The crude mixture was purified by preparative hplc using CH₃CN/5% acetonitrile in water buffer containing 0.1 M ammonium acetate to give 60 mg (68% yield) of the title compound as a light yellow powder after evaporation of solvent and freeze drying over night.

¹H NMR (400 MHz, methanol-d₄ as solvent and internal reference) δ(ppm) 1.19 (m, 2H), 1.54 (broad d, 1H, J=12.9 Hz), 1.66 (broad d, 1H, J=12.9 Hz), 1.75 (m, 1H), 2.51 (m, 1H), 2.63 (m, 2H), 3.07 (m, 1H), 3.42 (m, 2H), 3.49-3.94 (m, 14H), 4.04 (d, 1H, J=16.7 Hz), 4.64 (m, 1H), 6.79 (d, 1H, J=9.8), 7.38 (d, 1H, J=9.8 Hz), 7.51 (m, 1H), 7.57 (s, 1H), 7.73 (d, 1H, J=8.2 Hz), 7.90 (s, 1H).

The enantiomers i) and ii) were separated by preparative chiral chromatography.

i) HRMS (ESI+) calc. [M+H]⁺ 632.2058, found 632.2092.

ii) HRMS (ESI+) calc. [M+H]⁺ 632.2058, found 632.2092.

### EXAMPLE 33

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropylamide

### i) (R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid Isopropylamide and ii) (S)-4-(3-Chloro-1H-indole-6-sulfonyl)-1-[1-(1-methyl-6-oxo-1,6-dihydro pyridazin-3-yl)-piperidin-4-ylmethyl]-6-oxo-piperazine-2-carboxylic acid Isopropylamide

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid, the title product of Example 1 (54 mg, 0.096 mmol) was dissolved in 1 ml dry N,N-dimethylformamide, diisopropyl-ethylamine (0.031 mL, 0.18 mmol) and 2-(1H-benzotriazole-1-yl)-1,1,3,3-tetramethyl-uronium tetrafluoroborate (34 mg, 0.11 mmol) was added. The mixture was stirred for 5 minutes at room temperature before N,N-diisopropylamine (0.030 mL, 0.35 mmol) was added. The reaction mixture was stirred over night. More N,N-diisopropylethylamine (0.10 mL, 0.57 mmol), 2-(1H-benzotriazole-1-yl)-1,1,3,3-tetramethyluronium tetrafluoroborate (31 mg, 0.096 mmol) and isopropylamine (0.10 mL, 1.2 mmol) was added. After 2 days part of the solvent was evaporated in vacuo and the crude was purified by preparative HPLC using a gradient of CH₃CN/5% CH₃CN in water phase containing 0.1 M ammonium acetate to give 31 mg (53% yield) of the desired title compound as a white powder after evaporation of solvent and freeze drying over night.

¹H NMR (400 MHz, methanol-d₄ as solvent and internal reference) δ(ppm) 1.11-1.24 (m, 8H), 1.49 (broad d, 1H, J=12.8 Hz), 1.62 (broad d, 1H, J=12.8 Hz), 1.74 (m, 1H), 2.59 (m, 3H), 3.15 (m, 1H), 3.46 (d, 1H, J=16.1 Hz), 3.58 (s, 3H), 3.73-3.87 (m, 4H), 3.93-4.01 (m, 2H), 4.08 (m, 1H), 6.79 (d, 1H, J=9.8 Hz), 7.38 (d, 1H, J=9.8 Hz), 7.51 (m, 1H), 7.57 (s, 1H), 7.73 (d, 1H, J=8.5 Hz), 7.90 (s, 1H).

The enantiomers i) and ii) were separated by preparative chiral chromatography.

i) HRMS (ESI+) calc. [M+H]⁺ 590.1953, found 590.1964.

### EXAMPLE 34

### 6-{4-[2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one

### i) 6-{4-[(R)-2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one and ii) 6-{4-[(S)-2-(Azetidine-1-carbonyl)-4-(3-chloro-1H-indole-6-sulfonyl)-6-oxo-piperazin-1-ylmethyl]-piperidin-1-yl}-2-methyl-2H -pyridazin-3-one

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid, the title product of Example 1 (62 mg, 0.11 mmol), was dissolved in 1.1 mL N,N-dimethylformamide, N,N-diisopropyl-ethylamine (0.038 mL, 0.22 mmol) and 2-(1H-benzotriazole-1-yl)-1,1,3,3-tetramethyl-uronium tetrafluoroborate (39 mg, 0.12 mmol) was added. The mixture was stirred for 5 minutes before azetidine (0.03 mL, 0.44 mmol) was added. The reaction mixture was stirred over night. More N,N-diisopropylethylamine (0.1 mL, 0.57 mmol), 2-(1H-benzo-triazole-1-yl)-1,1,3,3-tetramethyluronium tetrafluoroborate (40 mg, 0.12 mmol) and azetidine (0.03 mL, 0.44 mmol) was added. After 2 days, part of the solvent was evaporated in vacuo and the crude was purified by preparative HPLC using a gradient of CH₃CN/5% CH₃CN in water phase containing 0.1 M ammonium acetate to give 39 mg (58% yield) of the desired title compound as a light yellow powder after evaporation of solvent and freeze drying over night.

¹H NMR (400 MHz, methanol-d₄ as solvent and internal reference) δ(ppm) 1.17 (m, 2H), 1.50 (broad d, 1H, J=12.2 Hz), 1.62 (broad d, 1H, J=12.2 Hz), 1.73 (m, 1H), 2.25 (m, 2H), 2.59 (m, 3H), 3.18 (m, 1H), 3.54 (d, 1H, J=16.4 Hz), 3.58 (s, 3H), 3.72-3.86 (m, 5H), 4.00 (m, 2H), 4.14 (m, 1H), 4.21-4.31 (m, 2H), 6.80 (d, 1H, J=10.1 Hz), 7.38 (d, 1H, J=10.1 Hz), 7.55 (m, 1H), 7.58 (s, 1H), 7.75 (d, 1H, 8.8 Hz), 7.93 (s, 1H).

The enantiomers i) and ii)were isolated by preparative chiral chromatography.

i) HRMS (ESI+) calc. [M+H]⁺ 602.1953, found 602.1948.

ii) HRMS (ESI+) calc. [M+H]⁺ 602.1953, found 602.1958.

### EXAMPLE 35

### 6-{4-[4-(3-Chloro-1H-indole-6-sulfonyl)-2-hydroxymethyl-6-oxo-piperazin-1-ylmethyl]-piperidine-1-yl}-2-methyl-2H-pyridazin-3-one

### A) 6-{4-[4-(1-Benzenesulfonyl-3-chloro-1H-indole-6-sulfonyl)-2-hydroxymethyl-6-oxo-piperazin-1-ylmethyl]-piperidin-1-yl}-2-methyl-2H-pyridazin-3-one

4-(1-Benzenesulfonyl-3-chloro-1H-indole-6-sulfonyl)-1[1-(1-methyl-6-oxo-1,6-dihydro-pyridazine-3-yl)-piperidin-4-ylmethyl]-6-oxo-piperazine-2 carboxylic acid, the title product of Example 1 (30 mg, 0.040 mmol) was dissolved in tetrahydrofuran (5 ml) together with triethylamine (5 mg, 0.05 mmol). The reaction mixture was cooled on an ice/salt bath to −18° C. and isobutyl chloroformate (6 mg, 0.05 mmol) was added. After 30 minutes the formed precipitate was filtered off and the reaction mixture was cooled again to −18° C. Sodium borohydride (5 mg, 0.13 mmol) was added and a few drops of water. When the foaming was over another 2 mL of water was added and the reaction mixture was allowed to stand at ambient temperature for 1 hour. Water was added, tetrahydrofuran was removed in vacuo and the remaining water phase was extracted three times with dichloromethane. The combined organic phase was washed with water and brine, dried with sodium sulfate and after filtration the solvent was evaporated in vacuo to give 30 mg of the sub-title compound which was used without further purification in the next step.

### B)

The intermediate was dissolved in tetrahydrofuran (2 mL) and lithium hydroxide (2 mg, 0.09 mmol) dissolved in water (1 mL) was added. The reaction mixture was allowed to stand at ambient temperature for 2 hours whereupon the pH was adjusted to 5-6 by addition of 0.1 M hydrochloric acid. Water (20 mL) was added, tetrahydrofuran was removed in vacuo and the remaining water phase was extracted three times with dichloromethane (20 mL). The combined organic phase was washed with water and brine, dried with sodium sulfate and the solvent evaporated in vacuo. The residue was purified by HPLC (Kromasil C8) using a gradient of acetonitrile (20-70% in water containing 0.1 M ammonium acetate to give 4.5 mg of the title compound after evaporation and freeze drying.

¹H NMR (400 MHz, methanol-d₄ as solvent and internal reference) δ (ppm): 1.04-1.20 (m, 1H), 1.19-1.30 (m, 1H), 1.45-1.52 (broad d, 1H), 1.58-1.65 (broad d, 1H), 1.81-1.90 (m, 1H), 2.62 (q, 2H, J=12 Hz), 2.80-2.91 (m, 2H), 3.38 (d, 1H, J=17.6 Hz), 3.46-3.52 (m, 1 H), 3.59 (s, 3H), 3.67-3.76 (m, 2H), 3.77-3.89 (m, 3H), 3.90-3.98 (m, 2H), 6.81 (d, 1H, J=10 Hz), 7.38 (d, 1H, J=10 Hz), 7.55 (d, 1H, J=8.8 Hz), 7.58 (s, 1H), 7.76 (d, 1H, J=8.8 Hz), 7.94 (s, 1H).

HRMS (ESI+) calc. [M+H]⁺ 549.1687, found 549.1686.

### EXAMPLE 36

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide

### i) (R-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide and ii) (S)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid, the title product of EXAMPLE 1, (40 mg, 0.071 mmol) was dissolved in 1 mL dry N,N-dimethylformamide and 2-(1H-benzotriazole-1-yl)-1,1,3,3-tetramethyluronium tetrafluoroborate (91 mg, 0.28 mmol, 4 equiv.) was added. The mixture was stirred for 5 minutes at room temperature before 2-methoxy-ethylamine (0.031 ml, 0.36 mmol) was added. The reaction mixture was stirred for 1 hour. The crude material was purified by preparative HPLC using a gradient of CH₃CN/5% CH₃CN in water phase containing 0.1 M ammonium acetate to give 40 mg (91% yield) of the desired title compound as a white powder after evaporation of solvent and freeze drying over night.

¹H NMR (500 MHz, methanol-d₄ as solvent and internal reference) δ(ppm) 1.10-1.27 (m, 2 H), 1.50 (broad d, 1 H, J=13 Hz), 1.63 (broad d, 1 H, J=13 Hz), 1.72-1.82 (m, 1 H), 2.55-2.69 (m, 3 H), 3.14-3.20 (m, 1 H), 3.35 (s, 3 H), 3.36-3.52 (m, 5 H), 3.59 (s, 3 H), 3.77-3.92 (m, 4 H), 4.01 (d, 1 H, J=17 Hz), 4.14-4.18 (m, 1 H), 6.81 (d, 1 H, J=10 Hz), 7.40 (d, 1 H, J=10 Hz), 7.51-7.55 (m, 1 H), 7.59 (s, 1 H), 7.75 (d, 1 H, J=9 Hz), 7.92 (s, 1 H).

The enantiomers i) and ii) were isolated by preparative chiral chromatography.

i) HRMS (ESI+) calc. [M+H]⁺ 620.2058, found 602.2055.

ii) HRMS (ESI+) calc. [M+H]⁺ 620.2058, found 602.2056.

### EXAMPLE 37

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid tert-butyl ester

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (50 mg, 0.089 mmol) was suspended in dry toluene (1.5 ml). N,N-dimethylformamide di-tert-butyl acetal (72 mg, 0.36 mmol, 4 equiv.) was added dropwise before the reaction mixture was heated at 85° C. (oil bath temperature). One equivalent of N,N-dimethylformamide di-tert-butyl acetal was added dropwise. The reaction mixture was stirred for an additional hour. This procedure was repeated twice. The reaction mixture was cooled and concentrated under reduced pressure before purification by prep-HPLC using a gradient of CH₃CN/5% CH₃CN in a water phase containing 0.1 M ammonium acetate to give 15 mg (27% yield) of the desired title compound as a white powder after evaporation of solvent and freeze drying over night.

¹H NMR (500 MHz, dimethyl sulfoxide-d₆ as solvent and internal reference) δ(ppm) 0.96-1.17 (m, 2 H), 1.43-1.48 (m, 10 H), 1.57 (broad d, 1 H, J=14 Hz), 1.62-1.72 (m, 1 H), 2.46-2.58 (m, 3 H), 2.90 (dd, 1 H, J=3, 12 Hz), 3.22 (d, 1 H, J=16 Hz), 3.44 (s, 3 H), 3.63-3.82 (m, 4 H), 4.00 (d, 1 H, J=12 Hz), 4.24-4.27 (m, 1 H), 6.75 (d, 1 H, J=10 Hz), 7.41 (d, 1 H, J=10 Hz), 7.48 (dd, 1 H, J=2, 8 Hz), 7.72 (d, 1 H, J=8 Hz), 7.87 (d, 1 H, J=1 Hz), 7.88 (s, 1 H).

HRMS (ESI+) calc. [M+H]⁺ 619.2106, found 619.207.

### EXAMPLE 38

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid ethyl ester

To a reaction vial containing 4-(3-chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (12 mg, 0.021 mmol) was added hydrochloric acid-saturated ethanol. The reaction vial was equipped with a septum and the reaction mixture was heated at 70° C. for 90 min. The reaction mixture was evaporated to dryness under reduced pressure before the crude was dissolved in dimethyl sulfoxide and purified by preparative HPLC using a gradient of CH₃CN/5% CH₃CN in a water phase containing 0.1 M ammonium acetate to give 12 mg (95% yield) of the desired title compound as a white powder after evaporation of solvent and freeze drying over night.

¹HNMR (500 MHz, acetonitrile-d₃ as solvent and internal reference) δ(ppm) 1.08 (dq, 1 H, J=4, 12 Hz), 1.18 (dq, 1 H J=4, 12 Hz), 1.25 (t, 3 H, J=7 Hz), 1.49 (broad d, 1 H, J=13 Hz), 1.59 (broad d, 1 H, J=13 Hz), 1.63-1.73 (m, 1 H), 2.50-2.59 (m, 3 H), 2.94 (dd, 1 H, J=3, 12 Hz), 3.32 (d, 1 H, J=16 Hz), 3.48 (s, 3 H), 3.65-3.76 (m, 2 H), 3.81 (dd, 1 H, J=8, 14 Hz), 3.93 (d, 1 H, J=16 Hz), 4.10 (dm, 1 H, J=12 Hz), 4.12-4.24 (m, 3 H), 6.67 (d, 1 H, J=10 Hz), 7.18 (d, 1 H, J=10 Hz), 7.52 (dd, 1 H, J=1, 8 Hz), 7.57 (d, 1 H, J=3 Hz), 7.74 (d, 1 H, J=8 Hz), 7.95 (s, 1 H), 9.96 (s, 1 NH).

HRMS (ESI+) calc. [M+H]⁺ 591.1793, found 591.1782.

### EXAMPLE 39

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid Isopropyl ester

To a reaction vial containing 4-(3-chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (180 mg, 0.32 mmol) was added hydrochloric acid-saturated propan-2-ol. The reaction vial was equipped with a septum and the reaction mixture was heated at 85° C. for 2.5 h. The reaction mixture was evaporated to dryness under reduced pressure before the crude was dissolved in dimethyl sulfoxide and purified by preparative HPLC using a gradient of CH₃CN/5% CH₃CN in a water phase containing 0.1 M ammonium acetate to give 144 mg (74% yield) of the desired title compound as a white powder after evaporation of solvent and freeze drying over night.

¹H NMR (500 MHz, dimethyl sulfoxide-d₆ as solvent and internal reference) δ(ppm) 1.00 (dq, 1 H, J=4, 12 Hz), 1.12 (dq, 1 H J=4, 12 Hz), 1.24 (dd, 6 H, J=2, 6 Hz), 1.46 (broad d, 1 H, J=12 Hz), 1.57 (broad d, 1 H, J=12 Hz), 1.64-1.74 (m, 1 H), 2.48-2.59 (m, 3 H), 2.95 (dd, 1 H, J=3, 12 Hz), 3.25 (d, 1 H, J=16 Hz), 3.44 (s, 3 H), 3.65-3.77 (m, 3 H), 3.79 (dd, 1 H, J=16 Hz), 4.00 (d, 1 H, J=12 Hz), 4.33-4.36 (m, 1 H), 4.98 (sept., 1 H, J=6 Hz), 6.75 (d, 1 H, J=10 Hz), 7.41 (d, 1 H, J=10 Hz), 7.47 (dd, 1 H, J=2, 8 Hz), 7.72 (d, 1 H, J=8 Hz), 7.87 (d, 1 H, J=1 Hz), 7.88 (s, 1 H).

HRMS (ESI+) calc. [M+H]⁺ 605.1949, found 605.1946.

### EXAMPLE 40

### 6-[4-({4-[(5-chloro-1H-indol-2-yl)sulfonyl]piperazin-1-yl}carbonyl)piperidin-1-yl]pyridazin-3(2H)-one

### A) 5-Chloro-2-[(4-{[1-(6-chloropyridazin-3-yl)piperidin-4-yl]carbonyl}piperazin-1-yl)sulfonyl]-1H-indole

1-(3-Dimethylaminopropyl)-3-ethoxycarbodiimide hydrochloride (590 mg, 3.08 mmol) was added to a solution of 5-chloro-2-(piperazin-1-ylsulfonyl)-1H-indole (770 mg, 2.57 mmol), 1-hydroxybenztriazole (470 mg, 3.08 mmol) and 1-(6-chloropyridazin-3-yl)piperidine-4-carboxylic acid (620 mg, 2.57 mmol) in N,N-dimethylformamide (10 mL) and stirred for 64 hours. The solvent was removed in vacuo and the resultant residue dissolved in dichloromethane (25 mL), washed with 25 mL of saturated sodium bicarbonate solution and then evaporated to leave a brown oil. The crude product was purified by chromatography on silica gel eluting with 0 to 3% methanol in dichloromethane to give 1.26 g of the sub-title compound (94%).

¹H NMR (400 Hz, dimethyl sulfoxide-d₆ as solvent and internal reference) δ(ppm) 1.43 (m, 2H), 1.60 (m, 2H), 2.89 (m, 3H), 2.97 (s, 4H), 3.52 (s, 2H), 3.62 (s, 2H), 4,23 (d, 2H), 7.00 (s, 1H), 7.30 (m, 2H), 7.45 (t, 2H), 7.76 (d, 1H).

### B)

5-Chloro-2-[(4-{[1-(6-chloropyridazin-3-yl)piperidin-4-yl]carbonyl}piperazin-1-yl)sulfonyl]-1H-indole, i.e. the sub-title compound from step A, (375 mg, 0.717 mmol) was added to a solution of potassium acetate (250 mg, 2.50 mmol) in glacial acetic acid (6.25 mL) and water (1.25 mL) and heated at 120° C. for 16 hours. After cooling to room temperature the solvents were removed in vacuo and the resulting residue stirred in water for 2 hours. The crude product was collected by filtration and purified by chromatography on silica eluting with 0 to 5% methanol in dichloromethane to give 135 mg of the title compound (37%).

¹H NMR (400 Hz, dimethyl sulphoxide-d₆ as solvent and internal reference) δ(ppm) 1.54 (m, 4H), 2.67 (m, 4H), 2.98 (s, 3H), 3.58 (m, 4H), 3.74 (d, 2H), 6.71 (d, 1H), 7.02 (s, 1H), 7.32 (dd, 1H), 7.46 (t, 2H), 7.77 (d, 1H), 12.05 (s, 1H), 12.45 (s, 1H).

HRMS (ESI+) calc. [M+H]⁺ 505.1420, found 505.1395.

1. A compound of formula (I) 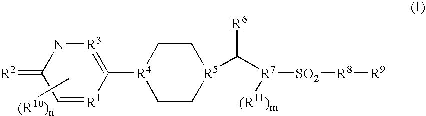
wherein
R¹ and R³, are independently selected from carbon and nitrogen;
R² is oxo or thioxo;
n is 0, 1 or 2;
each R¹⁰ is independently selected from hydrogen and C₁₋₃alkyl;
R⁴ and R⁵ are each selected from carbon and nitrogen, wherein at least one of R⁴ and R⁵ is nitrogen;
R⁶ is hydrogen or oxo;
R⁷ is an aliphatic, partially saturated or aromatic carbocyclic ring, said carbocyclic ring having 0, 1 or 2 hetero nitrogen;
m is 0, 1 or 2;
each R¹¹ is independently selected from hydrogen, hydroxy, oxo, C₁₋₅alkyl, carboxy, hydroxyC₁₋₅alkyl, carboxyC₁₋₅alkyl, C₁₋₅alkoxyoxoC₁₋₅alkyl, carbamoyl, C₁₋₅alkylcarbamoyl, di(C₁₋₅alkyl)carbamoyl, carbamoylC₁₋₄alkyl, C₁₋₅alkylcarbamoylC₁₋₄alkyl, di(C₁₋₅alkyl)carbamoylC₁₋₄alkyl, hydroxyC₁₋₅alkylcarbamoyl, C₁₋₅alkoxyC₁₋₅alkylcarbamoyl, hydroxyC₁₋₅alkylcarbamoylC₁₋₄alkyl, C₁₋₅alkoxyC₁₋₅alkylcarbamoylC₁₋₄alkyl, —CONR⁸⁰(CH₂)ₓS(O)ₚR⁹⁰, —CONH(CH₂)_{q}NR¹⁰⁰R¹¹⁰, —C₁₋₅alkyl-Y¹, —COOCHR¹⁷⁰R¹⁸⁰ and —CONR¹⁷⁰R¹⁸⁰:
wherein x represents an integer 0 to 4;
p is 0, 1 or 2;
q represents an integer 2 to 4;
R⁸⁰ represents hydrogen or C₁₋₃alkyl;
R⁹⁰ represents C₁₋₅alkyl or phenyl; or
R⁸⁰ and R⁹⁰ may together form a C₁₋₅alkylene group;
R¹⁰⁰ and R¹¹⁰ independently represent hydrogen, C₁₋₅alkyl, phenyl, C₁₋₅alkylphenyl, S(O)ₚR⁹⁰, COR¹²⁰ or a 5- or 6-membered monocyclic heteroaryl ring containing up to 3 heteroatoms selected from nitrogen, oxygen and sulphur;
R¹²⁰ represents hydrogen, C₁₋₅alkyl or phenyl;
Y¹ represents S(O)ₚR⁹⁰, NHS(O)₂R⁹⁰, NHCOR¹³⁰, O(CH₂)ᵣR¹⁴⁰, azetidino, pyrrolidin-1-yl, piperidino, morpholino, thiamorpholino, 1-oxothiamorpholino, 1,1-dioxothiamorpholino, piperazin-1-yl or C₁₋₅alkylamino,
R¹³⁰ represents C₁₋₅alkyl, phenyl or C₁₋₅alkylphenyl;
r represents an integer 1 to 4;
when r represents an integer 2 to 4, R¹⁴⁰ represents hydroxy, C₁₋₅alkylalkoxy, carboxy, C₁₋₅alkoxycarbonyl, S(O)ₚR⁹⁰ or NR¹⁵⁰R¹⁶⁰; and when r represents 1, R¹⁴⁰ represents carboxy or C₁₋₅alkoxycarbonyl;
wherein any phenyl group within R¹¹ is independently substituted by 0, 1 or 2 substituents selected from halogeno, trifluoromethyl, cyano, C₁₋₅alkyl and C₁₋₅alkoxy;
R¹⁵⁰ and R¹⁶⁰ independently represent hydrogen or C₁₋₅alkyl;
R¹⁷⁰ and R¹⁸⁰ are independently selected from hydrogen, C₁₋₆alkyl, C₄₋₇cycloalkyl, C₂₋₆alkenyl, R¹⁷⁰ and R¹⁸⁰ may form, along with the carbon to which they are attached, a 4-, 5-, 6- or 7-membered carbocyclic ring which contains 0, 1 or 2 heteroatoms selected from nitrogen, oxygen and sulphur, or R¹⁷⁰ and R¹⁸⁰ may form, along with the nitrogen to which they are attached, a 4-, 5-, 6- or 7-membered heterocyclic ring which contain in addition to the nitrogen atom present 0, 1 or 2 additional heteroatoms selected from nitrogen, oxygen and sulphur, wherein each R¹⁷⁰, R¹⁸⁰ or any of said rings formed by R¹⁷⁰ and R¹⁸⁰ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl;
R⁸ is a bond, C₁₋₄alkylene or C₂₋₆alkenylene;
R⁹ is an aromatic ring system having 0, 1 or 2 hetero atoms;
wherein R⁹ is substituted by 0 or 1 halogen;
or a pharmaceutically acceptable salt thereof.

2. A compound according to claim 1 wherein R¹ is nitrogen.

3. A compound according to claim 1 wherein R³ is nitrogen.

4. A compound according to claim 1 wherein R² is oxo.

5. A compound according to claim 1 wherein n is 0 or 1.

6. A compound according to claim 1 wherein one of R¹⁰ is hydrogen.

7. A compound according to claim 1 wherein one of R¹⁰ is C₁₋₃alkyl.

8. A compound according to claim 1 wherein R⁴ is nitrogen.

9. A compound according to claim 1 wherein R⁵ is nitrogen.

10. A compound according to claim 1 wherein both R⁴ and R⁵ are nitrogen.

11. A compound according to claim 1 wherein R⁶ is hydrogen.

12. A compound according to claim 1 wherein R⁶ is oxo.

13. A compound according to claim 1 wherein R⁷ is an aliphatic carbocyclic ring.

14. A compound according to claim 1 wherein R⁷ is a partially saturated carbocyclic ring.

15. A compound according to claim 1 wherein R⁷ is an aromatic carbocyclic ring.

16. A compound according to claim 1 wherein said carbocyclic ring has 0 hetero nitrogen.

17. A compound according to claim 1 wherein said carbocyclic ring has 1 hetero nitrogen.

18. A compound according to claim 1 wherein said carbocyclic ring has 2 hetero nitrogens.

19. A compound according to claim 1 wherein R⁷ is a carbocyclic ring of formula (Ia) 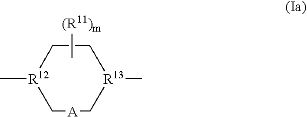
wherein A is a single bond or a double bond, and said hetero nitrogen or nitrogens is/are positioned at R¹² and/or R¹³ wherein R¹¹, R¹², R¹³, and m are as defined in claim 1.

20. A compound according to claim 19 wherein A is a single bond.

21. A compound according to claim 19 wherein said hetero nitrogens are positioned at R¹² and R¹³, respectively.

22. A compound according to claim 19 wherein said hetero nitrogen is positioned at R¹³.

23. A compound according to claim 1 where each R¹¹ is independently selected from hydrogen, hydroxy, oxo, C₁₋₅alkyl, carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl, C₁₋₅alkoxyC₁₋₅alkylcarbamoyl, —COOCHR¹⁷⁰R¹⁸⁰ and —CON R¹⁷⁰R¹⁸⁰:
wherein
R¹⁷⁰ and R¹⁸⁰ are independently selected from hydrogen, C₁₋₆alkyl, C₄₋₇cycloalkyl, C₂₋₆alkenyl, R¹⁷⁰ and R¹⁸⁰ may form, along with the carbon to which they are attached, a 4-, 5-, 6- or 7-membered carbocyclic ring which contains 0, 1 or 2 heteroatoms selected from nitrogen, oxygen and sulphur, or R¹⁷⁰ and R¹⁸⁰ may form, along with the nitrogen to which they are attached, a 4-, 5-, 6- or 7-membered heterocyclic ring which contain in addition to the nitrogen atom present 0, 1 or 2 additional heteroatoms selected from nitrogen, oxygen and sulphur, wherein each R¹⁷⁰, R¹⁸⁰ or any of said rings formed by R¹⁷⁰ and R¹⁸⁰ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl.

24. A compound according to claim 1, wherein one R¹¹ is oxo, and at least one further R¹¹ is selected from hydroxy, oxo, C₁₋₅alkyl, carboxy, hydroxyC₁₋₅alkyl, carboxyC₁₋₅alkyl, C₁₋₅alkoxyoxoC₁₋₅alkyl, carbamoyl, C₁₋₅alkylcarbamoyl, di(C₁₋₅alkyl)carbamoyl, carbamoylC₁₋₄alkyl, C₁₋₅alkylcarbamoylC₁₋₄alkyl, di(C₁₋₅alkyl)carbamoylC₁₋₄alkyl, hydroxyC₁₋₅alkylcarbamoyl, C₁₋₅alkoxyC₁₋₅alkylcarbamoyl, hydroxyC₁₋₅alkylcarbamoylC₁₋₄alkyl, C₁₋₅alkoxyC₁₋₅alkylcarbamoylC₁₋₄alkyl, —CONR⁸⁰(CH₂)ₓS(O)ₚR⁹⁰, —CONH(CH₂)_{q}NR¹⁰⁰R¹¹⁰, —C₁₋₅alkyl-Y¹, —COOCHR¹⁷⁰R¹⁸⁰ and —CONR¹⁷⁰R¹⁸⁰:
wherein x represents an integer 0 to 4;
p is 0, 1 or 2;
q represents an integer 2 to 4;
R⁸⁰ represents hydrogen or C₁₋₃alkyl;
R⁹⁰ represents C₁₋₅alkyl or phenyl; or
R⁸⁰ and R⁹⁰ may together form a C₁₋₅alkylene group;
R¹⁰⁰ and R¹¹⁰ independently represent hydrogen, C₁₋₅alkyl, phenyl, C₁₋₅alkylphenyl, S(O)ₚR⁹⁰, COR¹²⁰ or a 5- or 6-membered monocyclic heteroaryl ring containing up to 3 heteroatoms selected from nitrogen, oxygen and sulphur;
R¹²⁰ represents hydrogen, C₁₋₅alkyl, phenyl or C₁₋₅alkylphenyl;
Y¹ represents S(O)ₚR⁹⁰, NHS(O)₂R⁹⁰, NHCOR¹³⁰, O(CH₂)ᵣR¹⁴⁰, pyrrolidin-1-yl, piperidino, morpholino, thiamorpholino, 1-oxothiamorpholino, 1,1-dioxothiamorpholino or piperazin-1-yl,
R¹³⁰ represents C₁₋₅alkyl, phenyl or C₁₋₅alkylphenyl;
r represents an integer 1 to 4;
when r represents an integer 2 to 4, R¹⁴⁰ represents hydroxy, C₁₋₅alkylalkoxy, carboxy, C₁₋₅alkoxycarbonyl, S(O)ₚR⁹⁰ or NR¹⁵⁰OR¹⁶⁰; and when r represents 1, R¹⁴⁰ represents carboxy or C₁₋₅alkoxycarbonyl;
wherein any phenyl group within R¹¹ is independently substituted by 0, 1 or 2 substituents selected from halogeno, trifluoromethyl, cyano, C₁₋₅alkyl and C₁₋₅alkoxy;
R¹⁵⁰ and R¹⁶⁰ independently represent hydrogen or C₁₋₅alkyl;
R¹⁷⁰ and R¹⁸⁰ are independently selected from hydrogen, C₁₋₆alkyl, C₄₋₇cycloalkyl, C₂₋₆alkenyl, R¹⁷⁰ and R¹⁸⁰ may form along with the carbon to which they are attached a 4-, 5-, 6- or 7-membered carbocyclic ring which contains 0, 1 or 2 heteroatoms selected from nitrogen, oxygen and sulphur, or R¹⁷⁰ and R¹⁸⁰ may form along with the nitrogen to which they are attached a 4-, 5-, 6- or 7-membered heterocyclic ring which contain in addition to the nitrogen atom present 0, 1 or 2 additional heteroatoms selected from nitrogen, oxygen and sulphur, wherein each R¹⁷⁰, R¹⁸⁰ or any of said rings formed by R¹⁷⁰ and R¹⁸⁰ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl.

25. A compound according to claim 24, wherein said at least one further R¹¹ is selected from hydroxy, C₁₋₃alkyl, carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl, C₁₋₅alkoxyC₁₋₅alkylcarbamoyl, —CONR⁸⁰(CH₂)ₓS(O)ₚR⁹⁰, —CONH(CH₂)_{q}NR¹⁰⁰R¹¹⁰, —C₁₋₅alkyl-Y¹, —COOCHR¹⁷⁰R¹⁸⁰ and —CONR¹⁷⁰R¹⁸⁰:
wherein x represents an integer 0 to 4;
p is 0, 1 or 2;
q represents an integer 2 to 4;
R⁸⁰ represents hydrogen or C₁₋₃alkyl;
R⁹⁰ represents C₁₋₅alkyl or phenyl; or
R⁸⁰ and R⁹⁰ may together form a C₁₋₅alkylene group;
R¹⁰⁰ and R¹¹⁰ independently represent hydrogen, C₁₋₅alkyl, phenyl, C₁₋₅alkylphenyl, S(O)ₚR⁹⁰, COR¹²⁰ or a 5- or 6-membered monocyclic heteroaryl ring containing up to 3 heteroatoms selected from nitrogen, oxygen and sulphur;
R¹²⁰ represents hydrogen, C₁₋₅alkyl, phenyl or C₁₋₅alkylphenyl;
Y¹ represents S(O)ₚR⁹⁰, NHS(O)₂R⁹⁰, NHCOR¹³⁰, O(CH₂)ᵣR¹⁴⁰, pyrrolidin-1-yl, piperidino, morpholino, thiamorpholino, 1-oxothiamorpholino, 1,1-dioxothiamorpholino or piperazin-1-yl,
R¹³⁰ represents C₁₋₅alkyl, phenyl or C₁₋₅alkylphenyl;
r represents an integer 1 to 4;
when r represents an integer 2 to 4, R¹⁴⁰ represents hydroxy, C₁₋₅alkylalkoxy, carboxy, C₁₋₅alkoxycarbonyl, S(O)ₚR⁹⁰ or NR¹⁵⁰R¹⁶⁰; and when r represents 1, R¹⁴⁰ represents carboxy or C₁₋₅alkoxycarbonyl;
wherein any phenyl group within R¹¹ is independently substituted by 0, 1 or 2 substituents selected from halogeno, trifluoromethyl, cyano, C₁₋₅alkyl and C₁₋₅alkoxy;
R¹⁵⁰ and R¹⁶⁰ independently represent hydrogen or C₁₋₅alkyl;
R¹⁷⁰ and R¹⁸⁰ are independently selected from hydrogen, C₁₋₆alkyl, C₄₋₇cycloalkyl, C₂₋₆alkenyl, R¹⁷⁰ and R¹⁸⁰ may form along with the carbon to which they are attached a 4-, 5-, 6- or 7-membered carbocyclic ring which contains 0, 1 or 2 heteroatoms selected from nitrogen, oxygen and sulphur, or R¹⁷⁰ and R¹⁸⁰ may form along with the nitrogen to which they are attached a 4-, 5-, 6- or 7-membered heterocyclic ring which contain in addition to the nitrogen atom present 0, 1 or 2 additional heteroatoms selected from nitrogen, oxygen and sulphur, wherein each R¹⁷⁰, R¹⁸⁰ or any of said rings formed by R¹⁷⁰ and R¹⁸⁰ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl.

26. A compound according to claim 25, wherein said at least one further R¹¹ is selected from hydroxy, C₁₋₃alkyl, carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl, C₁₋₅alkoxyC₁₋₅alkylcarbamoyl, —COOCHR¹⁷⁰R¹⁸⁰ and —CONR¹⁷⁰R¹⁸⁰:
R¹⁷⁰ and R¹⁸⁰ are independently selected from hydrogen, C₁₋₆alkyl, C₄₋₇cycloalkyl, C₂₋₆alkenyl, R¹⁷⁰ and R¹⁸⁰ may form along with the carbon to which they are attached a 4-, 5-, 6- or 7-membered carbocyclic ring which contains 0, 1 or 2 heteroatoms selected from nitrogen, oxygen and sulphur, or R¹⁷⁰ and R¹⁸⁰ may form along with the nitrogen to which they are attached a 4-, 5-, 6- or 7-membered heterocyclic ring which contain in addition to the nitrogen atom present 0, 1 or 2 additional heteroatoms selected from nitrogen, oxygen and sulphur, wherein each R¹⁷⁰, R¹⁸⁰ or any of said rings formed by R¹⁷⁰ and R¹⁸⁰ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl.

27. A compound according to claim 24, wherein said at least one further R¹¹ is selected from C₁₋₃alkyl, carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl and C₁₋₅alkoxyC₁₋₅alkylcarbamoyl.

28. A compound according to claim 24, wherein said at least one further R¹¹ is selected from —COOCHR¹⁷⁰R¹⁸⁰ and —CON R¹⁷⁰ R¹⁸⁰:
R¹⁷⁰ and R¹⁸⁰ are independently selected from hydrogen, C₁₋₆alkyl, C₄₋₇cycloalkyl, C₂₋₆alkenyl, R¹⁷⁰ and R¹⁸⁰ may form along with the carbon to which they are attached a 4-, 5-, 6- or 7-membered carbocyclic ring which contains 0, 1 or 2 heteroatoms selected from nitrogen, oxygen and sulphur, or R¹⁷⁰ and R¹⁸⁰ may form along with the nitrogen to which they are attached a 4-, 5-, 6- or 7-membered heterocyclic ring which contain in addition to the nitrogen atom present 0 or 1 additional hetero oxygen, wherein each R¹⁷⁰, R¹⁸⁰ or any of said rings formed by R¹⁷⁰ and R¹⁸⁰ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl.

29. A compound according to claim 1 wherein R⁶ is oxo.

30. A compound according to claim 29 wherein each R¹¹ is independently selected from hydrogen, hydroxy, C₁₋₃alkyl, carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl, and C₁₋₅alkoxyC₁₋₅alkylcarbamoyl.

31. A compound according to claim 30 wherein one R¹¹ is hydroxy.

32. A compound according to claim 1 wherein m is 0.

33. A compound according to claim 1 wherein R⁸ is a bond.

34. A compound according to claim 1 wherein R⁸ is a C₂₋₄alkenylene.

35. A compound according to claim 1 wherein R⁹ is an aromatic ring system having 0, 1 or 2 hetero atoms, which hetero atoms are independently selected from nitrogen, oxygen and sulphur.

36. A compound according to claim 1 wherein said aromatic ring system is an aromatic ring.

37. A compound according to claim 36 wherein said aromatic ring has 1 hetero sulphur.

38. A compound according to claim 1 wherein said aromatic ring system is a fused bicyclic system comprising at least one benzene ring.

39. A compound according to claim 38 wherein said fused bicyclic system has 0 hetero atom.

40. A compound according to claim 38 wherein said fused bicyclic system has 1 hetero nitrogen.

41. A compound according to claim 1 wherein R⁹ is substituted by 0 or 1 halogen.

42. A compound according to claim 1 which is
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid,
(R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid methyl ester,
6-{4-[4-(3-Chloro-1H-indole-6-sulfonyl)-3-hydroxy-piperazine-1-carbonyl]-piperidin-1-yl}-2-methyl-2H-pyridazin-3-one,
6-{4-[4-(3-Chloro-1H-indole-6-sulfonyl)-2-hydroxy-piperazine-1-carbonyl]-piperidin-1-yl}-2-methyl-2H-pyridazin-3-one,
6-{4-[4-(3-Chloro-1H-indole-6-sulfonyl)-2-oxo-piperazin-1-ylmethyl]-piperidin-1-yl}-2-methyl-2H-pyridazin-3-one,
4-[4-(3-Chloro-1H-indole-6-sulfonyl)-piperazine-1-carbonyl]-5′-methyl-3,4,5,6-tetrahydro-2H,1′H-[1,3′]bipyridinyl-6′-one,
5-{4-[4-(3-Chloro-1H-indole-6-sulfonyl)-piperazine-1-carbonyl]-piperidin-1-yl}-3-methyl-1H-pyrazin-2-one,
6-{4-[4-(3-Chloro-1H-indole-6-sulfonyl)-piperazine-1-carbonyl]-piperidin-1-yl}-2-methyl-2H-pyridazin-3-one,
6-{4-[4-(1H-Indole-6-sulfonyl)-piperazine-1-carbonyl]-piperidin-1-yl}-2-methyl-2H-pyridazin-3-one,
6-{4-[4-(3-Chloro-1H-indole-6-sulfonyl)-piperazine-1-carbonyl]-piperidin-1-yl}-2H-pyridazin-3-one,
6-{4-[4-(3-Chloro-1H-indole-6-sulfonyl)-benzoyl]-piperazin-1-yl}-2-methyl-2H-pyridazin-3-one,
6-{4-[4-(6-Bromo-naphthalene-2-sulfonyl)-benzoyl]-piperidin-1-yl}-2-methyl-2H-pyridazin-3-one,
6-(4-{4-[(E)-2-(5-bromo-thiophen-2-yl)-ethenesulfonyl]-piperazine-1-carbonyl}-piperidin-1-yl)-2H-pyridazin-3-one,
6-(4-{4-[(E)-1-(5-chloro-thiophen-2-yl)-prop-1-ene-2-sulfonyl]-piperazine-1-piperidin-1-yl)-2H-pyridazin-3-one,
6-{1-[1-(5-chloro-1H-indole-2-sulfonyl)-piperidine-4-carbonyl]-piperazin-4-yl}-2-methyl-2H-pyridazin-3-one,
6-{1-[1-(5-Chloro-1H-indole-2-sulfonyl)-piperidine-4-carbonyl]-piperidin-4-yl}-2-methyl-2H-pyridazin-3-one,
6-(4-{(S)-4-[(E)-2-(5-chloro-thiophen-2-yl)-ethenesulfonyl]-2-methyl-6-oxo-piperazin-1-ylmethyl}-piperidin-1-yl)-2-methyl-2H-pyridazin-3-one,
6-(4-{(S)-4-[(E)-2-(5-chloro-thiophen-2-yl)-ethenesulfonyl]-5-methyl-2-oxo-piperazin-1-ylmethyl}-piperidin-1-yl)-2-methyl-2H-pyridazin-3-one,
(R)-4-(5-Chloro-1H-indole-2-sulfonyl)-1-[1-(1-methyl-6-oxo-1,6-dihydro-pyridazin-3-yl)-piperidin-4-ylmethyl]-6-oxo-piperazine-2-carboxylic acid methyl ester,
(R)-4-(5-Chloro-1H-indole-2-sulfonyl)-1-[1-(1-methyl-6-oxo-1,6-dihydro-pyridazin-3-yl)-piperidin-4-ylmethyl]-6-oxo-piperazine-2-carboxylic acid,
(R)-4-(6-Chloro-naphthalene-2-sulfonyl)-1-[1-(1-methyl-6-oxo-1,6-dihydro-pyridazin-3-yl)-piperidin-4-ylmethyl]-6-oxo-piperazine-2-carboxylic acid methyl ester,
(R)-4-(6-Chloro-naphthalene-2-sulfonyl)-1-[1-(1-methyl-6-oxo-1,6-dihydro-pyridazin-3-yl)-piperidin-4-ylmethyl]-6-oxo-piperazine-2-carboxylic acid,
(R)-4-[(E)-2-(5-Chloro-thiophen-2-yl)-ethenesulfonyl]-1-[1-(1-methyl-6-oxo-1,6-dihydro-pyridazin-3-yl)-piperidin-4-ylmethyl]-6-oxo-piperazine-2-carboxylic acid,
(R)-4-[(E)-2-(5-Chloro-thiophen-2-yl)-ethenesulfonyl]-1-[1-(1-methyl-6-oxo-1,6-dihydro-pyridazin-3-yl)-piperidin-4-ylmethyl]-6-oxo-piperazine-2-carboxylic acid methyl ester,
6-{4-[4-(6-Chloro-naphthalene-2-sulfonyl)-piperazine-1-carbonyl]-piperidin-1-yl}-2-methyl-2H-pyridazin-3-one,
6-(4-{4-[(E)-2-(5-Chloro-thiophen-2-yl)-ethenesulfonyl]-2-oxo-piperazin-1-ylmethyl}-piperidin-1-yl)-2-methyl-2H-pyridazin-3-one,
6-{4-[4-(5-Chloro-1H-indole-2-sulfonyl)-piperazine-1-carbonyl]-piperidin-1-yl}-2-methyl-2H-pyridazin-3-one,
6-(4-{4-[(E)-2-(5-Chloro-thiophen-2-yl)-ethenesulfonyl]-piperazine-1-carbonyl}-piperidin-1-yl)-2-methyl-2H-pyridazin-3-one,
6-{4-[4-(5-Chloro-1H-indole-2-sulfonyl)-3-hydroxy-piperazine-1-carbonyl]-piperidin-1-yl}-2-methyl-2H-pyridazin-3-one,
6-{4-[4-(3-Chloro-1H-indole-6-sulfonyl)-3,4-dihydro-2H-pyrazine-1-carbonyl]piperidin-1-yl}-2-methyl-2H-pyridazin-3-one,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid dimethylamide,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid ethylamide,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-hydroxy-ethyl)-amide,
6-{4-[4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one,
6-{4-[(R)-4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one,
6-{4-[(S)-4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropylamide,
(R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropylamide,
(S)-4-(3-Chloro-1H-indole-6-sulfonyl)-1-[1-(1-methyl-6-oxo-1,6-dihydro pyridazin-3-yl)-piperidin-4-ylmethyl]-6-oxo-piperazine-2-carboxylic acid isopropylamide,
6-{4-[2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one,
6-{4-[(R)-2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one,
6-{4-[(S)-2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one,
6-{4-[4-(3-Chloro-1H-indole-6-sulfonyl)-2-hyrdoxymethyl-6-oxo-piperazin-1-ylmethyl]-piperidine-1-yl}-2-methyl-2H-pyridazin-3-one,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide,
(R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide,
(S)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid tert-butyl ester,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid ethyl ester,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropyl ester, or
6-[4-({4-[(5-chloro-1H-indol-2-yl)sulfonyl]piperazin-1-yl}carbonyl)piperidin-1-yl]pyridazin-3(2H)-one.

43. A process for preparing a compound of formula (I) as defined in claim 1 which process comprises either
(a) reacting an amine of formula (II), 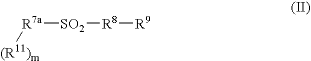
wherein R⁷a is a secondary amine part of a saturated or partially saturated heterocycle, and wherein R⁸, R⁹, R¹¹ and m are as defined in claim 1, with a carboxylic acid of the formula (III); 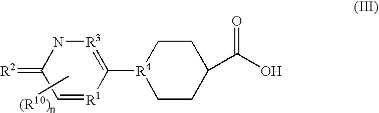
wherein R¹, R², R³, R⁴, R¹⁰, and n are as defined in claim 1;
(b) reacting a carboxylic acid derivative of formula (IV), or a suitably reactive derivative thereof 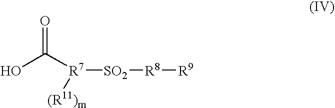
wherein R⁷, R⁸, R⁹, R¹¹, and m are as defined in claim 1, with an amine such as (V); 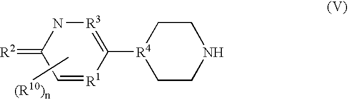
wherein R¹, R², R³, R⁴, R¹⁰, and n are as defined in claim 1;
(c) oxidative cleaving the exocyclic double bond of formula (VII); 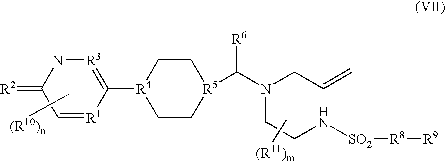
wherein R¹, R², R⁴, R⁵, R⁶, R⁸, R⁹, R¹⁰, R¹¹, m, and n are as defined in claim 1;
(d) preparing a compound of formula (VIII), 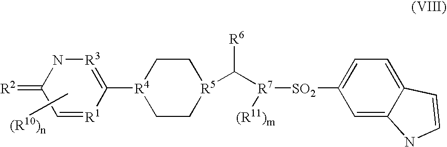
wherein R¹, R², R³, R⁴, R⁵, R⁶, R⁷, R¹⁰, R¹¹, m, and n are as defined in claim 1, and wherein the indolyl ring is substituted at C-3 by a halogen, from compounds of formula (VIII) by using the corresponding halogen succinimide;
(e) reacting an amine derivative of formula (XI), 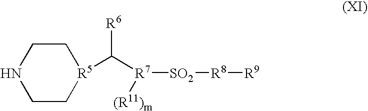
wherein R⁵, R⁶, R⁷, R⁸, R⁹, R¹¹, and m are as defined in claim 1, with a structure of formula (XII) 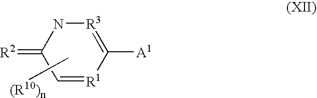
wherein R¹, R², R³, R⁸, R¹⁰, and n are as defined in claim 1, and wherein A¹ denotes a leaving group;
(f) reacting a sulfonyl chloride derivative of formula (XIV), 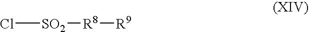
wherein R⁸ and R⁹ are as defined in claim 1, with an amine of formula (XV); 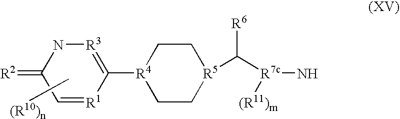
wherein R¹, R², R³, R⁴, R⁵, R⁶, R^{7c}, R¹⁰, R¹¹, m and n are as defined in claim 1;
(g) amide derivatives from the exocyclic carboxylic acid of formula (XVI), or a reactive derivative thereof, 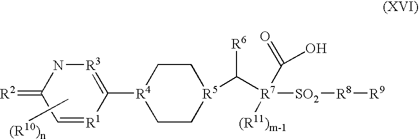
wherein R¹, R², R³, R⁴, R⁵, R⁶, R⁷, R⁸, R⁹, R¹⁰, R¹¹, m, and n are as defined in claim 1, are prepared using conditions described under (a);
(h) an ester derivative from the exocyclic carboxylic acid of formula (XVI) or a reactive derivative thereof, are prepared using acid catalysis, and using in case of hindered alcohols N,N-dimethylformamide dialkyl acetal;
(i) treating compounds of formula (VI) in acidic conditions;
(j) treating compounds of formula (XIX), 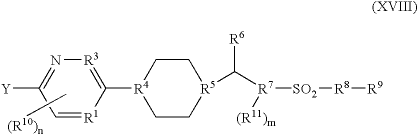
wherein Y is a halogen in acidic conditions; or
(k) oxidative cleavaging of the exocyclic double bond of formula (XX), 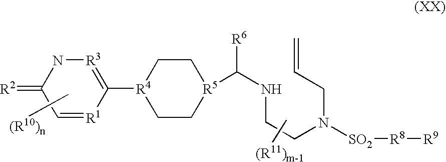
wherein R¹, R², R³, R⁴, R⁵, R⁶, R⁸, R⁹, R¹⁰ , R¹¹, m, and n are as defined in claim 1, as (c) above.

44. (canceled)

45. A pharmaceutical composition comprising a compound of formula (I), or a pharmaceutically-acceptable salt thereof, as defined in claim 1, with a pharmaceutically-acceptable diluent or carrier.

46. (canceled)

47. A method of treating a Factor Xa mediated disease or condition in a warm-blooded animal comprising administering an effective amount of a compound of formula (I), as defined in claim 1, or a pharmaceutically-acceptable salt thereof.

48. A combination comprising a compound of formula (I), as defined in claim 1, or a pharmaceutically-acceptable salt thereof, and one or more antithrombotic agent(s) with a different mechanism of action, wherein said antithrombotic agent(s) is selected from: an anticoagulant, a vitamin K antagonist, a synthetic or biotechnological inhibitor of other coagulation factors than FXa, an antiplatelet agent; a thromboxane receptor and/or synthetase inhibitor; a fibrinogen receptor antagonist; a prostacyclin mimetic; a phosphodiesterase inhibitor; an ADP-receptor antagonist; and an inhibitor of carboxypeptidase U and an inhibitor of plasminogen activator inhibitor-1 (PAI-1).

49. A combination comprising a compound of formula (I), as defined in claim 1, or a pharmaceutically-acceptable salt thereof, and a thrombolytic agent.

50. A process according to claim 43 wherein:
the indolyl ring in (d) is substituted at C-3 by a chloro or bromo;
A¹ in (e) denotes halogen;
the acid catalysis in (h) is saturation of the solvent by gaseous hydrochloric acid; and
Y in (j) is chloro or bromo.

51. A combination according to claim 48 wherein:
the anticoagulant is unfractionated heparin, low molecular weight heparin, other heparin derivative, or synthetic heparin derivative;
the synthetic or biotechnological inhibitor of other coagulation factors than FXa is an inhibitor of synthetic thrombin, FVIIa, FXIa, FIXa, or rNAPc2;
the antiplatelet agent is acetylsalicylic acid, ticlopidine, or clopidogrel;
the ADP-receptor antagonist is an antagonist of P2X1, P2Y1, P2Y12, or P2T; and
the carboxypeptidase U is CPU or TAFIa.

52. A combination according to claim 51 wherein the synthetic heparin derivative is fondaparinux.

53. A combination according to claim 49 wherein the thrombolytic agent is selected from a tissue plasminogen activator, streptokinase, urokinase, prourokinase, anisoylated plasminogen-streptokinase activator complex (APSAC), and an animal salivary gland plasminogen activator.
